SEQUENCE LISTING (111 sequences)

SEQ ID NO: 1 - protein, 137 aa, Homo sapiens
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 111 aa, Homo sapiens
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLA

SEQ ID NO: 4 - protein, 247 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 5 - protein, 111 aa, Artificial Sequence
  Mutant form of human CD86 extracellular domain
APLKIQAYFNETADLPCQFANSQNQSLSELVVFWQDQENLVLNEVYLGKEKFDSVASKYMGRTSFDSDSWTLRLHNLQIKDKGLYQCIIHHKKPTGMIRIHQMNSELSVLA

SEQ ID NO: 6 - protein, 109 aa, Artificial Sequence
  CD86 mutant 900
LKIQAYFNETADLPCQFANSQNQSLSELVVFWQDQENLVLNEVYLGKEKFDSVDSKYMGRTSFDSDSWTLRLHNLQIKDKGIYQCVIHHKKPSGLVKIHEMNSELSVLA

SEQ ID NO: 7 - protein, 109 aa, Artificial Sequence
  CD86 mutant 901
LKIQAYFNETADLPCQFANSQNLTLSELVVFWQDQENLVLNEVYLGKEKFDSVHSKYMGRTSFDSDSWTLRLHNLQIKDKGIYQCVIHHKKPTGMIKIHEMNSELSVLT

SEQ ID NO: 8 - protein, 109 aa, Artificial Sequence
  CD86 mutant 904
LKIQAYFNETADLPCQFANSQNQSLSELIVFWQDQENLVLNEVYLGKERFDAVDSKYMGRTSFDSDSWTLRLHNLQIKDKGIYQCIIHHKKPSGMVKIHQMDSELSVLA

SEQ ID NO: 9 - protein, 109 aa, Artificial Sequence
  CD86 mutant 906
LKIQAYINETADLPCQFANSQNLSLSELVVFWQDQENLVLNEVYLGKERFDSVDSKYMGRTSFDSDSWTLRLHNLQIKDKGFYQCIIHHKKPTGLVKIHEMNSELSVLA

SEQ ID NO: 10 - protein, 109 aa, Artificial Sequence
  CD86 mutant 907
LKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIKIHEMNSELSVLA

SEQ ID NO: 11 - protein, 109 aa, Artificial Sequence
  CD86 mutant 908
LKIQAYFNETADLPCQFANSQNQSLSELVVFWQDQENLVLNEVYLGKEKFDSVHSKYMGRTSFDSDSWTLRLHNLQIKDKGIYQCIIHHKKPTGMVKIHEMNSELSVLA

SEQ ID NO: 12 - protein, 109 aa, Artificial Sequence
  CD86 mutant 910
LKIQAYFNETADLPCQFANSQNQSLSELVVFWQDQENLVLNEVYLGKEKFDSVDSKYMGRTSFDSDSWTLRLHNLQIKDKGIYQCIIHHKKPTGMVKIHEMNSELSVLA

SEQ ID NO: 13 - protein, 109 aa, Artificial Sequence
  CD86 mutant 915
LKIQAYFNETADLPCQFANSQNQSLSELVVFWQDQENLILNEVYLGKEKFDSVDSKYMGRTSFDSDSWTLRLHNLQIKDKGFYQCIIHHKKPSGLIKIHQMDSELSVLA

SEQ ID NO: 14 - protein, 109 aa, Artificial Sequence
  CD86 mutant 938
LKIQAYFNETADLPCQFANSQNQSLSELVVFWQDQENLILNEVYLGKEKFDSVHSKYMGRTSFDSDSWTLRLHNLQIKDKGIYQCIIHHKKPTGMVKIHQMNSELSVLA

SEQ ID NO: 15 - protein, 111 aa, Artificial Sequence
  CD86 mutant 1038
APLKIQAYFNETADLPCQFANSQNLSLSELVVFWQDQENLVLNEVYLGKEKFDSVDSKYMGRTSFDSDSWTLRLHNLQIKDKGIYQCIIHHKKPTGMVKIHEMNSELSVLA

SEQ ID NO: 16 - protein, 111 aa, Artificial Sequence
  CD86 mutant 1039
APLKIQAYFNETADLPCQFANSQNLSLSELVVFWQDQENLVLNEVYLGKEKFDSVSSKYMGRTSFDSDSWTLRLHNLQIKDKGIYQCIIHHKKPSGMVKIHQMDSELSVLA

SEQ ID NO: 17 - protein, 111 aa, Artificial Sequence
  CD86 mutant 1040
APLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 18 - protein, 111 aa, Artificial Sequence
  CD86 mutant 1041
APLKIQAYLNETADLPCQFANSQNLSLSELVVFWQDQENLVLNEVYLGKEKFDSVDSKYMGRTSFDSDSWTLRLHNLQIKDKGIYQCIIHHKKPTGLVKIHEMNSELSVLA

SEQ ID NO: 19 - protein, 111 aa, Artificial Sequence
  CD86 mutant 1042
APLKIQAYFNETADLPCQFANSQNLSLSELVVFWQDQENLVLNEVYLGKEIFDSVSSKYMGRTSFDSDSWTLRLHNLQIKDKGIYQCIIHHKKPSGMVKIHQMDSELSVLA

SEQ ID NO: 20 - protein, 111 aa, Artificial Sequence
  CD86 mutant 1043
APLKIQAYFNETADLPCQFANSQNLSLSELVVFWQDQENLVLNEVYLGKEKFDSVDSKYMGRTSFDSDSWTLRLHNLQIKDKGIYQCIIHHKKPTGMIKIHEMNSELSVLA

SEQ ID NO: 21 - protein, 111 aa, Artificial Sequence
  CD86 mutant 1044
APLKIQAYFNETADLPCQFANSQNLTLSELVVFWQDQENLVLNEVYLGKEKFDSVSSKYMGRTSFDSDSWTLRLHNLQIKDKGIYQCIIHHKKPTGMIKIHEMSSELSVLA

SEQ ID NO: 22 - protein, 111 aa, Artificial Sequence
  CD86 mutant 1045
APLKIQAYFNETADLPCQFANSQNLTLSELVVFWQDQENLVLNEVYLGKEKFDSVDSKYMGRTSFDSDSWTLRLHNLQIKDKGLYQCIIHHKKPTGLVKIHEMNSELSVLA

SEQ ID NO: 23 - protein, 111 aa, Artificial Sequence
  CD86 mutant 1046
APLKIQAYFNETADLPCQFANSQNQSLSELVVFWQDQENLVLNEVYLGKEKFDSVDSKYMGRTSFDSDSWTLRLHNLQIEDKGIYQCIIHHKKPSGMVKIHQMDSELSVLA

SEQ ID NO: 24 - protein, 111 aa, Artificial Sequence
  CD86 mutant 1047
APLKIQAYFNETADLPCQFANSQNLSLSELVVFWQDQENLVLNEVYLGKEKFDSVDSKYMGRTSFDSDSWTLRLHNLQIKDKGIYQCIIHHKKPTGLVKIHEMNSELSVLA

SEQ ID NO: 25 - DNA, 327 bp, Artificial Sequence
  CD86 mutant 900
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcgtgatccaccataagaagccgagcggtctggtgaagattcacgagatgaactccgagttgtctgtcctggcg

SEQ ID NO: 26 - DNA, 327 bp, Artificial Sequence
  CD86 mutant 901
ctcaaaatccaagcgtacttcaacgaaactgcagacttaccgtgtcagtttgccaattcgcagaatctgaccctgagcgaactggtggttttctggcaggatcaggagaacctggttctgaacgaagtctatctgggcaaagagaaattcgacagcgtgcatagcaagtatatgggccgcaccagctttgatagcgacagctggaccctgcgtctgcacaatctgcaaatcaaagataagggtatctaccagtgcgtgatccaccataagaagccgacgggtatgattaagattcacgagatgaactccgagttgtctgtcctgacc

SEQ ID NO: 27 - DNA, 327 bp, Artificial Sequence
  CD86 mutant 904
ctcaaaatccaagcgtacttcaacgaaactgcagacttaccgtgtcagtttgccaattcgcagaatcaaagcctgagcgaactgatcgttttctggcaggatcaggagaacctggttctgaacgaagtctatctgggcaaagagcggttcgacgccgtggacagcaagtatatgggccgcaccagctttgatagcgacagctggaccctgcgtctgcacaatctgcaaatcaaagataagggtatctaccagtgcattatccaccataagaagccgagcggtatggtgaagattcaccaaatggactccgagttgtctgtcctggcg

SEQ ID NO: 28 - DNA, 327 bp, Artificial Sequence
  CD86 mutant 906
ctcaaaatccaagcgtacatcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtttctaccagtgcattatccaccataagaagccgacgggtctggtgaagattcacgagatgaactccgagttgtctgtcctggcg

SEQ ID NO: 29 - DNA, 327 bp, Artificial Sequence
  CD86 mutant 907
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtgcatagcaagtatatgggccgcaccagctttgatagcgacagctggaccctgcgtctgcacaatctgcaaatcaaagataagggtctgtaccagtgcattatccaccataagaagccgacgggtatgattaagattcacgagatgaactccgagttgtctgtcctggcg

SEQ ID NO: 30 - DNA, 327 bp, Artificial Sequence
  CD86 mutant 908
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtgcatagcaagtatatgggccgcaccagctttgatagcgacagctggaccctgcgtctgcacaatctgcaaatcaaagataagggtatctaccagtgcattatccaccataagaagccgacgggtatggtgaagattcacgagatgaactccgagttgtctgtcctggcg

SEQ ID NO: 31 - DNA, 327 bp, Artificial Sequence
  CD86 mutant 910
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtatggtgaagattcacgagatgaactccgagttgtctgtcctggcg

SEQ ID NO: 32 - DNA, 327 bp, Artificial Sequence
  CD86 mutant 915
ctcaaaatccaagcgtacttcaacgaaactgcagacttaccgtgtcagtttgccaattcgcagaatcaaagcctgagcgaactggtggttttctggcaggatcaggagaacctgatcctgaacgaagtctatctgggcaaagagaaattcgacagcgtggacagcaagtatatgggccgcaccagctttgatagcgacagctggaccctgcgtctgcacaatctgcaaatcaaagataagggtttctaccagtgcattatccaccataagaagccgagcggtctgattaagattcaccaaatggactccgagttgtctgtcctggcg

SEQ ID NO: 33 - DNA, 327 bp, Artificial Sequence
  CD86 mutant 938
ctcaaaatccaagcgtacttcaacgaaactgcagacttaccgtgtcagtttgccaattcgcagaatctgagcctgagcgaactggtggttttctggcaggatcaggagaacctgatcctgaacgaagtctatctgggcaaagagcggttcgacagcgtgcatagcaagtatatgggccgcaccagctttgatagcgacagctggaccctgcgtctgcacaatctgcaaatcaaagataagggtctgtaccagtgcattatccaccataagaagccgagcggtatggtgaagattcacgagatgaactccgagttgtctgtcctggcg

SEQ ID NO: 34 - DNA, 333 bp, Artificial Sequence
  CD86 mutant 1038
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtatggtgaagattcacgagatgaactccgagttgtctgtcctggcg

SEQ ID NO: 35 - DNA, 333 bp, Artificial Sequence
  CD86 mutant 1039
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtgagtagcaagtatatgggccgcaccagctttgatagcgacagctggaccctgcgtctgcacaatctgcaaatcaaagataagggtatctaccagtgcattatccaccataagaagccgagcggtatggtgaagattcaccaaatggactccgagttgtctgtcctggcg

SEQ ID NO: 36 - DNA, 333 bp, Artificial Sequence
  CD86 mutant 1040
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 37 - DNA, 333 bp, Artificial Sequence
  CD86 mutant 1041
gcccccctcaaaatccaagcgtacctcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtctggtgaagattcacgagatgaactccgagttgtctgtcctggcg

SEQ ID NO: 38 - DNA, 333 bp, Artificial Sequence
  CD86 mutant 1042
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagattttcgacagcgtgagtagcaagtatatgggccgcaccagctttgatagtgacagctggaccctgcgtctgcacaatctgcaaatcaaagataagggtatctaccagtgcattatccaccataagaagccgagcggtatggtgaagattcaccaaatggactccgagttgtctgtcctggcg

SEQ ID NO: 39 - DNA, 333 bp, Artificial Sequence
  CD86 mutant 1043
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggatagcaagtatatgggccgcaccagctttgatagcgacagctggaccctgcgtctgcacaatctgcaaatcaaagataagggtatctaccagtgcattatccaccataagaagccgacgggtatgattaagattcacgagatgaactccgagttgtctgtcctggcg

SEQ ID NO: 40 - DNA, 333 bp, Artificial Sequence
  CD86 mutant 1044
gcccccctcaaaatccaagcgtacttcaacgaaactgcagacttaccgtgtcagtttgccaattcgcagaatctgaccctgagcgaactggtggttttctggcaggatcaggagaacctggttctgaacgaagtctatctgggcaaagagaaattcgacagcgtgtctagcaagtatatgggccgcaccagctttgatagcgacagctggaccctgcgtctgcacaatctgcaaatcaaagataagggtatctaccagtgcattatccaccataagaagccgacgggtatgattaagattcacgagatgagctccgagttgtctgtcctggcg

SEQ ID NO: 41 - DNA, 333 bp, Artificial Sequence
  CD86 mutant 1045
gcccccctcaaaatccaagcgtacttcaacgaaactgcagacttaccgtgtcagtttgccaattcgcagaatctgaccctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtctgtaccagtgcattatccaccataagaagccgacgggtctggtgaagattcacgagatgaactccgagttgtctgtcctggcg

SEQ ID NO: 42 - DNA, 333 bp, Artificial Sequence
  CD86 mutant 1046
gcccccctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcgaagataagggtatctaccagtgcattatccaccataagaagccgagcggtatggtgaagattcaccaaatggactccgagttgtctgtcctggcg

SEQ ID NO: 43 - DNA, 333 bp, Artificial Sequence
  CD86 mutant 1047
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtctggtgaagattcacgagatgaactccgagttgtctgtcctggcg

SEQ ID NO: 44 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 45 - protein, 223 aa, Mus <mouse, genus>
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVSAVSLSKMLKKRSPLTTGVYVKMPPTEPECEKQFQPYFIPIN

SEQ ID NO: 46 - protein, 218 aa, Mus <mouse, genus>
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQVTTMNMTPRRPGLTRKPYQPYAPARDFAAYRP

SEQ ID NO: 47 - protein, 11 aa, Artificial Sequence
  Linker
SGGGGSGGGGS

SEQ ID NO: 48 - protein, 13 aa, Artificial Sequence
  Linker
SGGGGSGGGGSAP

SEQ ID NO: 49 - protein, 5 aa, Artificial Sequence
  Linker
NFSQP

SEQ ID NO: 50 - protein, 5 aa, Artificial Sequence
  Linker
KRTVA

SEQ ID NO: 51 - protein, 15 aa, Artificial Sequence
  Linker
GGGGSGGGGSGGGGS

SEQ ID NO: 52 - protein, 117 aa, Artificial Sequence
  2348, heavy chain VH
EVQLLESGGGLVQPGGSLRLSCAASGFTFGYYYMSWVRQAPGKGLEWVSGISSPSSYTYYADSVKGRFTISRDNSKNTLYLQMNSLRAEDTAVYYCARYYGSYFDYWGQGTLVTVSS

SEQ ID NO: 53 - DNA, 351 bp, Artificial Sequence
  2348, heavy chain VH
gaggtgcagctgttggagagcgggggaggcttggtacagcctggggggtccctgcgcctctcctgtgcagccagcggattcacctttggttactactacatgtcttgggtccgccaggctccagggaaggggctggagtgggtctcaggtatttcttctccttcttcttacacatactatgcagactccgtgaagggccggttcaccatctcccgtgacaattccaagaacacgctgtatctgcaaatgaacagcctgcgtgccgaggacacggctgtatattattgtgcgcgctactacggttcttactttgactattggggccagggaaccctggtcaccgtctcctca

SEQ ID NO: 54 - protein, 118 aa, Artificial Sequence
  2372 (VH)
EVQLLESGGGLVQPGGSLRLSCAASGFTFSGYSMGWVRQAPGKGLEWVSAISGSGGSTYYADSVKGRFTISRDNSKNTLYLQMNSLRAEDTAVYYCARYPWGYYFDYWGQGTLVTVSS

SEQ ID NO: 55 - DNA, 354 bp, Artificial Sequence
  2372 (VH)
gaggtgcagctgttggagagcgggggaggcttggtacagcctggggggtccctgcgcctctcctgtgcagccagcggattcaccttttctggttactctatgggttgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgctacccgtggggttactactttgactattggggccagggaaccctggtcaccgtctcctca

SEQ ID NO: 56 - protein, 121 aa, Artificial Sequence
  2396 (VH)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAYPVHGYWVFDYWGQGTLVTVSS

SEQ ID NO: 57 - DNA, 363 bp, Artificial Sequence
  2396 (VH)
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgcgcttacccggttcatggttactgggtttttgactattggggccagggaaccctggtcaccgtctcctca

SEQ ID NO: 58 - protein, 116 aa, Artificial Sequence
  2404 (VH)
EVQLLESGGGLVQPGGSLRLSCAASGFTFSYSSMSWVRQAPGKGLEWVSYIGSGGSHTYYADSVKGRFTISRDNSKNTLYLQMNSLRAEDTAVYYCARYSYYFDYWGQGTLVTVSS

SEQ ID NO: 59 - DNA, 348 bp, Artificial Sequence
  2404 (VH)
gaggtgcagctgttggagagcgggggaggcttggtacagcctggggggtccctgcgcctctcctgtgcagccagcggattcaccttttcttactcttctatgtcttgggtccgccaggctccagggaaggggctggagtgggtctcatacattggttctggtggttctcacacatactatgcagactccgtgaagggccggttcaccatctcccgtgacaattccaagaacacgctgtatctgcaaatgaacagcctgcgtgccgaggacacggctgtatattattgtgcgcgctactcttactactttgactattggggccagggaaccctggtcaccgtctcctca

SEQ ID NO: 60 - DNA, 327 bp, Artificial Sequence
  2349 (VL) (mAb - without CTLA-4 binding domain)
gacatccagatgacccagtctccatcctccctgagcgcatctgtaggagaccgcgtcaccatcacttgccgggcaagtcaggctattagcgcttatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtcttacggttactacctgtacacttttggccaggggaccaagctggagatcaaacgt

SEQ ID NO: 61 - protein, 108 aa, Artificial Sequence
  2349 (VL) (mAb - without CTLA-4 binding domain)
DIQMTQSPSSLSASVGDRVTITCRASQAISAYLNWYQQKPGKAPKLLIYAASSLQSGVPSRFSGSGSGTDFTLTISSLQPEDFATYYCQQSYGYYLYTFGQGTKLEIK

SEQ ID NO: 62 - DNA, 327 bp, Artificial Sequence
  2373 (VL) (mAb - without CTLA-4 binding domain)
gacatccagatgacccagtctccatcctccctgagcgcatctgtaggagaccgcgtcaccatcacttgccgggcaagtcagggtattagagcttatttaaattggtatcagcagaaaccagggaaagcccctaagctcctgatctatgctgtatccagtttgcaaagtggggtcccatcacgtttcagtggcagtggaagcgggacagatttcactctcaccatcagcagtctgcaacctgaagattttgcaacttattactgtcaacagtactactacccgccgctgtccacttttggccaggggaccaagctggagatcaaacgt

SEQ ID NO: 63 - protein, 108 aa, Artificial Sequence
  2373 (VL) (mAb - without CTLA-4 binding domain)
DIQMTQSPSSLSASVGDRVTITCRASQGIRAYLNWYQQKPGKAPKLLIYAVSSLQSGVPSRFSGSGSGTDFTLTISSLQPEDFATYYCQQYYYPPLSTFGQGTKLEIK

SEQ ID NO: 64 - DNA, 324 bp, Artificial Sequence
  2397 (VL) (mAb - without CTLA-4 binding domain)
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtctgtttctactccgcccacttttggccaggggaccaagctggagatcaaacgt

SEQ ID NO: 65 - protein, 107 aa, Artificial Sequence
  2397 (VL) (mAb - without CTLA-4 binding domain)
DIQMTQSPSSLSASVGDRVTITCRASQSISSYLNWYQQKPGKAPKLLIYAASSLQSGVPSRFSGSGSGTDFTLTISSLQPEDFATYYCQQSVSTPPTFGQGTKLEIK

SEQ ID NO: 66 - DNA, 327 bp, Artificial Sequence
  2405 (VL) (mAb - without CTLA-4 binding domain)
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagagtcattactggtacccgctcacttttggccaggggaccaagctggagatcaaacgt

SEQ ID NO: 67 - protein, 108 aa, Artificial Sequence
  2405 (VL) (mAb - without CTLA-4 binding domain)
DIQMTQSPSSLSASVGDRVTITCRASQSISSYLNWYQQKPGKAPKLLIYAASSLQSGVPSRFSGSGSGTDFTLTISSLQPEDFATYYCQQSHYWYPLTFGQGTKLEIK

SEQ ID NO: 68 - DNA, 1376 bp, Artificial Sequence
  2349 Light chain VL, with constant kappa sequence, linker and CD86 mutant 1040 inclusive intron sequence
gacatccagatgacccagtctccatcctccctgagcgcatctgtaggagaccgcgtcaccatcacttgccgggcaagtcaggctattagcgcttatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtcttacggttactacctgtacacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 69 - protein, 337 aa, Artificial Sequence
  2349 light chain VL, with constant kappa sequence, linker and CD86 mutant 1040
DIQMTQSPSSLSASVGDRVTITCRASQAISAYLNWYQQKPGKAPKLLIYAASSLQSGVPSRFSGSGSGTDFTLTISSLQPEDFATYYCQQSYGYYLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 70 - DNA, 1376 bp, Artificial Sequence
  2373 Light chain VL, with constant kappa sequence, linker and CD86 mutant 1040
gacatccagatgacccagtctccatcctccctgagcgcatctgtaggagaccgcgtcaccatcacttgccgggcaagtcagggtattagagcttatttaaattggtatcagcagaaaccagggaaagcccctaagctcctgatctatgctgtatccagtttgcaaagtggggtcccatcacgtttcagtggcagtggaagcgggacagatttcactctcaccatcagcagtctgcaacctgaagattttgcaacttattactgtcaacagtactactacccgccgctgtccacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 71 - protein, 337 aa, Artificial Sequence
  2373 Light chain VL, with constant kappa sequence, linker and CD86 mutant 1040
DIQMTQSPSSLSASVGDRVTITCRASQGIRAYLNWYQQKPGKAPKLLIYAVSSLQSGVPSRFSGSGSGTDFTLTISSLQPEDFATYYCQQYYYPPLSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 72 - DNA, 1373 bp, Artificial Sequence
  2397 Light chain VL, with constant kappa sequence, linker and CD86 mutant 1040
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtctgtttctactccgcccacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 73 - protein, 336 aa, Artificial Sequence
  2397 Light chain VL, with constant kappa sequence, linker and CD86 mutant 1040
DIQMTQSPSSLSASVGDRVTITCRASQSISSYLNWYQQKPGKAPKLLIYAASSLQSGVPSRFSGSGSGTDFTLTISSLQPEDFATYYCQQSVSTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 74 - DNA, 1376 bp, Artificial Sequence
  2405 Light chain VL, with constant kappa sequence, linker and CD86 mutant 1040
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagagtcattactggtacccgctcacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 75 - protein, 337 aa, Artificial Sequence
  2405 Light chain VL, with constant kappa sequence, linker and CD86 mutant 1040
DIQMTQSPSSLSASVGDRVTITCRASQSISSYLNWYQQKPGKAPKLLIYAASSLQSGVPSRFSGSGSGTDFTLTISSLQPEDFATYYCQQSHYWYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 76 - protein, 8 aa, Artificial Sequence
  2348 CDRH1
GFTFGYYY

SEQ ID NO: 77 - protein, 8 aa, Artificial Sequence
  2348 CDRH2
ISSPSSYT

SEQ ID NO: 78 - protein, 10 aa, Artificial Sequence
  2348 CDRH3
ARYYGSYFDY

SEQ ID NO: 79 - protein, 8 aa, Artificial Sequence
  2372 CDRH1
GFTFSGYS

SEQ ID NO: 80 - protein, 8 aa, Artificial Sequence
  2372 CDRH2
ISGYSMGT

SEQ ID NO: 81 - protein, 11 aa, Artificial Sequence
  2372 CDRH3
ARYPWGYYFDY

SEQ ID NO: 82 - protein, 8 aa, Artificial Sequence
  2396 CDRH1
GFTFSSYA

SEQ ID NO: 83 - protein, 8 aa, Artificial Sequence
  2396 CDRH2
ISGSGGST

SEQ ID NO: 84 - protein, 14 aa, Artificial Sequence
  2396 CDRH3
ARAYPVHGYWVFDY

SEQ ID NO: 85 - protein, 8 aa, Artificial Sequence
  2404 CDRH1
GFTFSYSS

SEQ ID NO: 86 - protein, 8 aa, Artificial Sequence
  2404 CDRH2
ISYSSMST

SEQ ID NO: 87 - protein, 9 aa, Artificial Sequence
  2404 CDRH3
ARYSYYFDY

SEQ ID NO: 88 - protein, 6 aa, Artificial Sequence
  2349 CDRL1
QAISAY

SEQ ID NO: 89 - skipped

SEQ ID NO: 90 - protein, 10 aa, Artificial Sequence
  2349 CDRL3
QQSYGYYLYT

SEQ ID NO: 91 - protein, 6 aa, Artificial Sequence
  2373 CDRL1
QGIRAY

SEQ ID NO: 92 - skipped

SEQ ID NO: 93 - protein, 10 aa, Artificial Sequence
  2373 CDRL3
QQYYYPPLST

SEQ ID NO: 94 - protein, 6 aa, Artificial Sequence
  2397 CDRL1 or 2405 CDRL1
QSISSY

SEQ ID NO: 95 - protein, 9 aa, Artificial Sequence
  2397 CDRL3
QQSVSTPPT

SEQ ID NO: 96 - protein, 10 aa, Artificial Sequence
  2405 CDRL3
QQSHYWYPLT

SEQ ID NO: 97 - protein, 330 aa, Homo sapiens [sequence omitted]

SEQ ID NO: 98 - protein, 107 aa, Homo sapiens [sequence omitted]

SEQ ID NO: 99 - protein, 327 aa, Artificial Sequence
  Modified IgG4 constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRYTQKSLSLSLGK

SEQ ID NO: 100 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 327 aa, Artificial Sequence
  Modified IgG4 constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - DNA, 981 bp, Artificial Sequence
  cDNA of the IgG4 constant region of SEQ ID NO: 99
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccgctacacacagaagagcctctccctgtctctgggtaaa

SEQ ID NO: 103 - DNA, 2028 bp, Artificial Sequence
  Genomic DNA of the IgG4 constant region of SEQ ID NO: 99
agctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccaccttgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccgctacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgagggaggcagagcgggtcccactgtccccacactgg

SEQ ID NO: 104 - DNA, 981 bp, Homo sapiens
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 105 - DNA, 2028 bp, Homo sapiens
agctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgagggaggcagagcgggtcccactgtccccacactgg

SEQ ID NO: 106 - DNA, 990 bp, Homo sapiens
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 107 - DNA, 1596 bp, Homo sapiens
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 108 - DNA, 321 bp, Homo sapiens
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 109 - DNA, 981 bp, Artificial Sequence
  cDNA of the IgG4 region of SEQ ID NO: 101
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 110 - DNA, 2028 bp, Artificial Sequence
  Genomic DNA of the IgG4 region of SEQ ID NO: 101
agctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccaccttgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgagggaggcagagcgggtcccactgtccccacactgg

SEQ ID NO: 111 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

(19) United States
(12) Patent Application Publication
(10) Pub. No.: US 2019/0309084 A1
(43) Pub. Date: Oct. 10, 2019

(54) Bispecific Polypeptides to GITR and CTLA-4

(71) Applicant: ALLIGATOR BIOSCIENCE AB, Lund (SE)

(72) Inventors: Peter Ellmark, Lund (SE); Sara Fritzell, Lund (SE); Christina Furebring, Lund (SE); Anne Kvarnhammar, Lund (SE); Mattias Levin, Lund (SE); Per Norlen, Lund (SE); Eva Nyblom, Lund (SE); Niina Veitonmaki, Lund (SE); Magnus Winnerstam, Lund (SE)

(21) Appl. No.: 16/461,525

(22) PCT Filed: Nov. 21, 2017
(86) PCT No.: PCT/EP2017/079925

§ 371 Date: May 16, 2019

(30) Foreign Application Priority Data
Nov. 21, 2016 (GB) 1619652.9

(51) Int. Cl. C07K 16/28; A61P 35/00; A61K 39/395

(52) U.S. Cl. CPC C07K 16/2878; A61P 35/00; C07K 16/2818; A61K 39/39558; A61K 39/39541; C07K 2317/52; A61K 2039/505; C07K 2317/92; C07K 2317/41; C07K 2317/734; C07K 2317/732; C07K 2317/565; C07K 2317/31

(57) ABSTRACT

The present invention provides multispecific polypeptides, such as bispecific antibodies, comprising a first binding domain capable of specifically binding to GITR, and a second binding domain capable of specifically binding to CTLA-4. The invention further provides compositions of said bispecific polypeptides, as well as methods and uses of the same.

### FIELD OF INVENTION

The present invention relates to multispecific (e.g. bispecific) polypeptides which specifically bind to GITR and CTLA-4, and use of the same in the treatment and prevention of cancer.

### BACKGROUND

Cancer is a leading cause of premature deaths in the developed world. Immunotherapy of cancer aims to mount an effective immune response against tumour cells. This may be achieved by, for example, breaking tolerance against tumour antigen, augmenting anti-tumour immune responses, and stimulating local cytokine responses at the tumour site.

The key effector cell of a long lasting anti-tumour immune response is the activated tumour specific effector T cell (T eff). Potent expansion of activated effector T cells can redirect the immune response towards the tumour. In this context, regulatory T cells (T reg) play a role in inhibiting the anti-tumour immunity. Depleting, inhibiting/reverting or inactivating Tregs may therefore provide anti-tumour effects and revert the immune suppression in the tumour microenvironment. Further, incomplete activation of effector T cells by, for example, dendritic cells can cause T cell anergy, which results in an inefficient anti-tumour response, whereas adequate induction by dendritic cells can generate a potent expansion of activated effector T cells, redirecting the immune response towards the tumour. In addition, Natural killer (NK) cells play an important role in tumour immunology by attacking tumour cells with down-regulated human leukocyte antigen (HLA) expression and by inducing antibody dependent cellular cytotoxicity (ADCC). Stimulation of NK cells may thus also reduce tumour growth.

Glucocorticoid-induced TNFR-related protein (GITR, CD357 or TNFRSF18) is an important co-stimulatory receptor for T cells that can potentiate T cell receptor (TCR) signaling during T cell priming of naïve CD4⁺ and CD8⁺ T cells, T cell effector (Teff) differentiation and memory T cell responses. In humans, GITR expression is generally low on naïve CD4+ and CD8+ T cells, and is restricted to activated T cells and regulatory T cells (Tregs). GITR upregulation occurs after 6 hs upon TCR activation and peaks within 24 h (Kanamuru, 2004). GITR activation is triggered by its ligand GITRL, mainly expressed on antigen presenting cells (APCs) and endothelial cells. Similar to other TNFR family members, GITR co-stimulation together with TCR signaling induces the activation of the NFκB pathway, resulting in enhanced cytokine release, such as IL-2, IFNγ, IL-4, but also IL-10 (Kanamuru, 2004), inhibits CD3-induced apoptosis (Nocentini, 1997) and promotes T cell survival, proliferation and expansion. GITR stimulation thereby favors CD4 effector T cell expansion, maturation and differentiation to a memory phenotype and CD8 T cell activation. Importantly, GITR is highly expressed on peripheral and thymic Tregs, especially on activated Tregs, where it plays an important but also contradictory role in their regulatory function (Ronchetti, 2015):
1) In mice models, GITR is crucial for Treg differentiation and expansion.
2) Conversely, GITR stimulation may abrogate Treg immunosuppressive function, for example via degradation of FOXP3 (Shimizu, 2002) (McHugh, 2002) (Cohen, 2010). This could partly by explained by a transient pharmacological effect due to overstimulation of GITR in non-physiological conditions.
3) GITR induced signaling may also promote T cells to become more resistant to immunosuppression induced by Tregs; enhancing T cell responsiveness to weakly immunogenic tumour associated antigens, leading to tumour directed immunity and tumour rejection.
4) Another suppressive effect of GITR antibodies on Tregs is dependent on the depletion of specifically Tregs, caused by binding of the GITR antibody Fc-part to activating Fcγ receptors (FcγR) and the higher expression of GITR on Tregs than on naïve T cells or Teffs. It has been suggested that this effect is restricted to the tumour area due to a high infiltration of FcγR-expressing natural killer cells (NK cells) and myeloid cells infiltrating the tumour (Bulliard, 2013).

The relative importance of these mechanisms for the therapeutic effect of GITR antibodies may be context dependent.

Currently there are eight GITR mAb in clinical development, in phase I. These include traditional bivalent monoclonal antibodies, but also MEDI-1873 (Medlmmune/AstraZeneca), a multivalent (hexamer) GITRL fusion protein coupled to an Fc domain, to maximize GITR multimerisation for optimal T cell activation and/or Treg depletion. TRX-518 (Leap Therapeutics), a humanized aglycosylated IgG1 GlTR antibody, is a non-depleting antibody that was the first to enter the clinic in 2010 against melanoma. The first single dose escalation study showed low efficacy or toxicity. A new dose escalation study with repeated dosing of TRX-518 opened in 2015. INCAGN01876 (Agenus/Incyte) and GWN323 (Novartis) are both IgG1 antibodies able to bind and activate FcγRs and induce ADCC of target cells, such as Tregs. At least four more GITR antibodies have reached clinical development from BMS, Amgen and Merck. The isotype of the antibodies and their abilities to induce ADCC will likely impact the balance of Treg depletion and T cell effector function as a mode of action for the different GITR targeting compounds.

The T cell receptor CTLA-4 serves as a negative regulator of T cell activation, and is upregulated on the T-cell surface following initial activation. The ligands of the CTLA-4 receptor, which are expressed by antigen presenting cells, are the B7 proteins, CD80 and CD86. The corresponding ligand receptor pair that is responsible for the upregulation of T cell activation is CD28-B7. Signalling via CD28 constitutes a costimulatory pathway, and follows upon the activation of T cells, through the T cell receptor recognizing antigenic peptide presented by the MHC complex. By blocking the CTLA-4 interaction to CD80/CD86, one of the normal check points of the immune response may be removed. The net result is enhanced activity of effector T cells which may contribute to anti-tumour immunity. This may be due to direct activation of the effector T cells but may also be due to a reduction in the activity and/or numbers of Treg cells, e.g. via ADCC or ADCP.

Check point blockade of CTLA-4 results in improved T cell activation and anti-tumour effects, but administration of anti-CTLA-4 antibodies has been associated with toxic side-effects. CTLA-4 is overexpressed on regulatory T cells in many solid tumours, such as melanoma lung cancer, renal cancer and head and neck cancer (Kwiecien, 2017) (Montler, 2016) (Ross, Clin Science, 2017).

Clinical studies with CTLA-4 antibody treatment (Ipilimumab) of melanoma have demonstrated a survival advantage (Nodi et al., 2010). The mechanisms of the effect of Ipilimumab, being an IgG1 antibody, has not been fully elucidated. Current data support a dual activity of CTLA-4 antibodies, activating peripheral Teffs and depleting intratumoural Tregs (Bulliard, 2013) (Furness, 2014).

By blocking the CTLA-4-CD80/CD86 interaction, one of the normal check points in the immune response is removed. This has the potential to result in undesired immune activation and even if it results in anti-tumour effects, it is also associated with toxic side effects. Others have demonstrated that local production of anti-CTLA-4 antibodies (by tumour cells) results in anti-tumour effect without autoimmune reactions associated with systemic administration (Fransen, 2013).

Ipilimumab (BMS), an anti-CTLA-4 mAb in IgG1 format, is approved for the treatment of melanoma and is currently in clinical phase III against for example non-small cell lung carcinoma (NSCLC), small cell lung cancer (SCLC), bladder and prostate cancer. In addition, BMS has a non-fucosylated version of Ipilimumab in clinical phase I. Tremelimumab, (Medlmmune/Astra Zeneca), is an anti-CTLA-4 IgG2 mAb in clinical phase III against for example mesothelioma, NSCLC and bladder cancer. AGEN-1884 (Agenus Inc.) is a recently enrolled anti-CTLA-4 antibody in phase I against advanced solid tumours.

Monospecific antibodies targeting GITR or CTLA-4 are in general dependent on cross linking via e.g. Fcγ receptors on other cells to induce a strong signaling into cells expressing the respective receptor. Thus, they do not signal efficiently when no such cross linking is provided.

There is a need for an alternative to the existing monospecific drugs that target only one T cell target, such as either of GITR or CTLA-4.

### SUMMARY OF INVENTION

A first aspect of the invention provides a multispecific polypeptide comprising a first binding domain, designated B1, which is capable of specifically binding to CTLA-4, and a second binding domain, designated B2, which is capable of specifically binding to GITR.

By “multispecific” polypeptides we include polypeptides capable of binding to more than one target epitope, typically on different antigens. Examples of such polypeptides include bispecific antibodies and trispecific antibodies, and polypeptide derivatives thereof (see below).

Thus, bispecific antibodies are molecules with the ability to bind to two different epitopes on the same or different antigens. Bispecific antibodies are developed to enable simultaneous inhibition of two cell surface receptors, or blocking of two ligands, cross-linking of two receptors or recruitment of T cells to the proximity of tumour cells (Fournier, 2013).

Multispecific antibodies targeting two or more different T cell targets, such as CTLA-4 and GITR, have the potential to specifically activate the immune system in locations where all targets are over expressed. For example, CTLA-4 is overexpressed on regulatory T cells (Treg) in the tumour microenvironment, whereas its expression on effector T cells is lower. Thus, the multispecific antibodies of the invention have the potential to selectively target regulatory T cells in the tumour microenvironment.

GITR expression is associated with CTLA-4 expression on activated Tregs known to infiltrate the tumour microenvironment, and their suppressive activity is correlated with GITR and CTLA-4 expression (Ronchetti, 2015) (Furness, 2014) (Bulliard, 2013) (Leving, 2002). The bispecific antibody has thus the potential to selectively target suppressive Tregs in the tumour and specifically deplete Tregs or reverse the immune suppression of Tregs. This effect could be mediated by ADCC or ADCP induction via the Fc part of the bispecific antibody (Furness, 2014) or by signaling induced via GITR stimulation and/or by blocking the CTLA-4 signaling pathway (Walker, 2011). On Teffs, the bispecific antibody has the potential to induce activation and increase effector function both via GITR stimulation and through CTLA-4 checkpoint blockade. A combination study of GITR stimulation and CTLA-4 blockade of ex vivo isolated Tregs from cancer patients show that immune suppression can be abrogated and restore T cell antitumour immunity (Gonzales, 2015). Furthermore, studies in mouse models suggest a beneficial anti-tumoural effect when combining GITR stimulation and CTLA-4 blockade (Pruitt, 2011).

In summary, and without wishing to be bound by theory, it is believed that the main mode of action of the multispecific (e.g. bispecific) antibody polypeptides of the invention is to deplete and suppress tumour infiltrating Tregs providing an enhanced effect compared with monospecific GITR antibodies while having a more tolerable safety profile compared with CTLA-4 antibodies such as Ipilimumab.

As multispecific antibodies, the GITR-CTLA-4 antibodies of the invention offer a potentially increased therapeutic efficacy, and an opportunity to reduce cost for drug development, production, clinical testing and regulatory approval in comparison to the combination of monospecific antibodies. The format per se may also give synergistic effects by physically linking two cells or two different cell receptors (May, 2012). These features make multispecific antibodies such as these very attractive as therapeutic agents in the treatment of cancer.

In particular, multispecific (e.g. bispecific) antibodies targeting GITR and CTLA-4 have the potential to activate the immune system locally in the tumour. As mentioned earlier, GITR and CTLA-4 expression is associated with activated Tregs known to infiltrate the tumour. The multispecific (e.g. bispecific) antibody has thus the potential to selectively target and specifically suppress or deplete Tregs (via ADCC) in the tumour. As a consequence, therapeutic efficacy is enhanced by dual binding to GITR and CTLA-4 in comparison with a bivalent binding of monospecific GITR or CTLA-4 antibodies, providing a beneficial anti-tumoural effect of the multispecific (e.g. bispecific) antibodies comparing to its monospecific competitors. Furthermore, the systemic dose of the multispecific (e.g. bispecific) antibodies may be lower than for a monospecific antibody, which can reduce toxicity and increase safety for the patients while simultaneously reducing costs.

The cell surface expression pattern of GITR and CTLA-4 is partly overlapping. A multispecific (e.g. bispecific) antibody targeting GITR and CTLA-4 has thus the potential to bind to both targets both in cis and in trans. Such bispecific antibody would potentially have the ability to stimulate through GITR and CTLA-4 in an FcγR-cross-linking independent manner, either by increasing the level of receptor clustering in cis on the same cell, or by creating an artificial immunological synapse between two cells, which in turn may lead to enhanced receptor clustering and increased signaling in both cells. Such cell-cell interactions lead to increased immune activation, which is not achieved by the combination of separate monospecific antibodies.

Thus, in exemplary embodiments, the multispecific (e.g. bispecific) polypeptides of the invention are capable of binding specifically to GITR and CTLA-4 thereby inducing:
1. A higher degree of immune activation compared to monospecific antibodies. The immune activation is significantly higher than the combination of CTLA-4 and GITR monospecific antibodies.
2. Activation also in the absence of any cross-linking, except for the cross-linking provided by the GITR and CTLA-4 binding entities, in contrast to the monospecific antibodies that only activate in the presence of cross-linking reagents, such as other cells expressing Fc gamma Receptors, physical cross-linking by adhering the antibodies to a surface, such as the well surface or cross-linking antibodies that binds to the Fc parts of the monospecific antibodies.
3. A more directed/localized immune activation. The immune activation only occurs in environments that contains both high GITR expression and CTLA-4 expression. The tumour microenvironment is such an environment. This has the potential to increase the effect and also to minimize toxic side effect. Thus, the therapeutic window may be increased.

A “polypeptide” is used herein in its broadest sense to refer to a compound of two or more subunit amino acids, amino acid analogues, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogues and peptidomimetics.

The term “multispecific” as used herein means the polypeptide is capable of specifically binding at least two different target entities, in this instance GITR and CTLA-4. Advantageously, the multispecific (e.g. bispecific) polypeptide of the invention is capable of binding to an extracellular domain of GITR and to an extracellular domain of CTLA-4. It will be appreciated that such binding specificity should be evident in vivo, i.e. following administration of the bispecific polypeptide to the patient.

In one embodiment, the first and/or second binding domains may be selected from the group consisting of: antibodies or antigen-binding fragments thereof.

As used herein, the terms “antibody” or “antibodies” refer to molecules that contain an antigen binding site, e.g. immunoglobulin molecules and immunologically active fragments of immunoglobulin molecules that contain an antigen binding site. Immunoglobulin molecules can be of any type (e.g. IgG, IgE, IgM, IgD, IgA and IgY), class (e.g. IgG1, IgG2, IgG3, IgG4, IgA1 and IgA2) or a subclass of immunoglobulin molecule. Antibodies include, but are not limited to, synthetic antibodies, monoclonal antibodies, single domain antibodies, single chain antibodies, recombinantly produced antibodies, multi-specific antibodies (including bi-specific antibodies), human antibodies, humanized antibodies, chimeric antibodies, intrabodies, scFvs (e.g. including mono-specific and bi-specific, etc.), Fab fragments, F(ab′) fragments, disulfide-linked Fvs (sdFv), anti-idiotypic (anti-Id) antibodies, and epitope-binding fragments of any of the above.

The terms antibody “directed to” or “directed against” are used interchangeably herein and refer to an antibody that is constructed to direct its binding specificity(ies) at a certain target/marker/epitope/antigen, i.e. an antibody that immunospecifically binds to a target/marker/epitope/antigen. Also, the expression antibodies “selective for” a certain target/marker/epitope may be used, having the same definition as “directed to” or “directed against”. A multispecific (e.g. bispecific) antibody directed to (selective for) at least two different targets/markers/epitopes/antigens binds immunospecifically to both targets/markers/epitopes/antigens. If an antibody is directed to a certain target antigen, such as GITR, it is thus assumed that said antibody could be directed to any suitable epitope present on said target antigen structure.

As used herein, the term “antibody fragment” is a portion of an antibody such as F(ab′).sub.2, F(ab).sub.2, Fab′, Fab, Fv, scFv and the like. Regardless of structure, an antibody fragment binds with the same antigen that is recognized by the intact antibody. For example, an anti-GITR antibody fragment binds to GITR. The term “antibody fragment” also includes isolated fragments consisting of the variable regions, such as the “Fv” fragments consisting of the variable regions of the heavy and light chains and recombinant single chain polypeptide molecules in which light and heavy variable regions are connected by a peptide linker (“scFv proteins”). As used herein, the term “antibody fragment” does not include portions of antibodies without antigen binding activity, such as Fc fragments or single amino acid residues.

ScFv domains are particularly preferred for inclusion in the multispecific (e.g. bispecific) antibodies of the invention.

Thus, in one embodiment the polypeptide is a multispecific (e.g. bispecific) antibody.

It will be appreciated by persons skilled in the art that the multispecific (e.g. bispecific) polypeptides of the invention may be of several different structural formats (for example, see Chan & Carter, 2016, Nature Reviews Immunology 10, 301-316, the disclosures of which are incorporated herein by reference).

In exemplary embodiments, the multispecific (e.g. bispecific) antibody is selected from the groups consisting of:
i) bivalent bispecific antibodies, such as IgG-scFv bispecific antibodies (for example, wherein B1 is an intact IgG and B2 is an scFv attached to B1 at the N-terminus of a light chain and/or at the C-terminus of a light chain and/or at the N-terminus of a heavy chain and/or at the C-terminus of a heavy chain of the IgG, or vice versa);
ii) monovalent bispecific antibodies, such as a DuoBody® (Genmab AS, Copenhagen, Denmark) or ‘knob-in-hole’ bispecific antibody (for example, an scFv-KIH, scFv-KIH^{r}, a BiTE-KIH or a BiTE-KIH^{r} (see Xu et al., 2015, mAbs 7(1):231-242);
iii) scFv₂-Fc bispecific antibodies (such as ADAPTIR™ bispecific antibodies from Emergent Biosolutions Inc);
iv) BiTE/scFv₂ bispecific antibodies;
v) DVD-lg bispecific antibodies;
vi) DART-based bispecific antibodies (for example, DART₂-Fc, DART₂-Fc or DART);
vii) DNL-Fab₃ bispecific antibodies; and
viii) scFv-HSA-scFv bispecific antibodies.

Thus, in exemplary embodiments of the multispecific (e.g. bispecific) antibodies of the invention:
(a) binding domain B1 and/or binding domain B2 is an intact IgG antibody (or, together, form an intact IgG antibody);
(b) binding domain B1 and/or binding domain B2 is an Fv fragment (e.g. an scFv);
(c) binding domain B1 and/or binding domain B2 is a Fab fragment; and/or
(d) binding domain B1 and/or binding domain B2 is a single domain antibody (e.g. domain antibodies and nanobodies).

For example, the multispecific (e.g. bispecific) antibody may be an IgG-scFv antibody. The IgG-scFv antibody may be in either VH-VL or VL-VH orientation. In one embodiment, the scFv may be stabilised by a S—S bridge between VH and VL.

In an alternative embodiment, the multispecific (e.g. bispecific) polypeptide of the invention may comprise a first binding domain which comprises or consists of an antibody variable domain or part thereof and a second binding domain which is not an antibody variable domain or part thereof. Thus, the first and/or second binding domains may be a non-antibody polypeptide. For example, B1 may comprise or consist of an IgG1 antibody and B2 may comprise or consist of a non-immunoglobulin polypeptide, or vice versa.

In one embodiment, B2 comprises or consists of a CD86 domain or variant thereof capable of binding to CTLA-4.

It will be appreciated by persons skilled in the art that binding domain B1 and binding domain B2 are fused directly to each other.

In an alternative embodiment, binding domain B1 and binding domain B2 are joined via a polypeptide linker. For example, a polypeptide linker may be a short linker peptide between about 10 to about 25 amino acids. The linker is usually rich in glycine for flexibility, as well as serine or threonine for solubility, and can either connect the N-terminus of the VH with the C-terminus of the VL, or vice versa. Exemplary linkers include a peptide of amino acid sequence as shown in any one of SEQ ID NOs. 47 to 51.

The multispecific (e.g. bispecific) polypeptides of the invention may be manufactured by any known suitable method used in the art. Methods of preparing bi-specific antibodies of the present invention include BiTE (Micromet), DART (MacroGenics), Fcab and Mabe (F-star), Fc-engineered IgG1 (Xencor) or DuoBody (based on Fab arm exchange, Genmab). Examples of other platforms useful for preparing bi-specific antibodies include but are not limited to those described in WO 2008/119353 (Genmab), WO 2011/131746 (Genmab) and reported by van der Neut-Kolfschoten et al. (2007, Science 317(5844):1554-7). Traditional methods such as the hybrid hybridoma and chemical conjugation methods (Marvin and Zhu (2005) Acta Pharmacol Sin 26: 649) can also be used. Co-expression in a host cell of two antibodies, consisting of different heavy and light chains, leads to a mixture of possible antibody products in addition to the desired bi-specific antibody, which can then be isolated by, e.g. affinity chromatography or similar methods.

It will be appreciated by persons skilled in the art that the multispecific (e.g. bispecific) antibody may comprise a human Fc region, or a variant of a said region, where the region is an IgG1, IgG2, IgG3 or IgG4 region, preferably an IgG1 or IgG4 region.

The constant (Fc) regions of the antibodies may mediate the binding of the immunoglobulin to host tissues or factors, including various cells of the immune system (e.g., effector cells) and the first component (Clq) of the classical complement system. The Fc region is preferably a human Fc region, or a variant of a said region. The Fc region may be an IgG1, IgG2, IgG3 or IgG4 region, preferably an IgG1 or IgG4 region. A variant of an Fc region typically binds to Fc receptors, such as FcγR and/or neonatal Fc receptor (FcRn) with altered affinity providing for improved function and/or half-life of the polypeptide. The biological function and/or the half-life may be either increased or a decreased relative to the half-life of a polypeptide comprising a native Fc region. Examples of such biological functions which may be modulated by the presence of a variant Fc region include antibody dependent cell cytotoxicity (ADCC), antibody-dependent cellular phagocytosis (ADCP), complement-dependent cytotoxicity (CDC), and/or apoptosis.

Thus, the Fc region may be naturally-occurring (e.g. part of an endogenously produced human antibody) or may be artificial (e.g. comprising one or more point mutations relative to a naturally-occurring human Fc region).

As is well documented in the art, the Fc region of an antibody mediates its serum half-life and effector functions, such as CDC, ADCC and ADCP.

Engineering the Fc region of a therapeutic monoclonal antibody or Fc fusion protein allows the generation of molecules that are better suited to the pharmacology activity required of them (Strohl, 2009, Curr Opin Biotechnol 20(6):685-91, the disclosures of which are incorporated herein by reference).

### (a) Engineered Fc Regions for Increased Half-Life

One approach to improve the efficacy of a therapeutic antibody is to increase its serum persistence, thereby allowing higher circulating levels, less frequent administration and reduced doses.

The half-life of an IgG depends on its pH-dependent binding to the neonatal receptor FcRn. FcRn, which is expressed on the surface of endothelial cells, binds the IgG in a pH-dependent manner and protects it from degradation.

Some antibodies that selectively bind the FcRn at pH 6.0, but not pH 7.4, exhibit a higher half-life in a variety of animal models.

Several mutations located at the interface between the CH2 and CH3 domains, such as T250Q/M428L (Hinton et al., 2004, J Biol Chem. 279(8):6213-6, the disclosures of which are incorporated herein by reference) and M252Y/S254T/T256E+H433K/N434F (Vaccaro et al., 2005, Nat. Biotechnol. 23(10):1283-8, the disclosures of which are incorporated herein by reference), have been shown to increase the binding affinity to FcRn and the half-life of IgG1 in vivo.

### (b) Engineered Fc Regions for Altered Effector Function

Depending on the therapeutic antibody or Fc fusion protein application, it may be desired to either reduce or increase the effector function (such as ADCC).

For antibodies that target cell-surface molecules, especially those on immune cells, abrogating effector functions may be required for certain clinical indications.

Conversely, for antibodies intended for oncology use (such as in the treatment of leukemias and solid tumours; see below), increasing effector functions may improve the therapeutic activity.

The four human IgG isotypes bind the activating Fcγ receptors (FcγRI, FcγRIIa, FcγRIIIa), the inhibitory FcγRIIb receptor, and the first component of complement (C1q) with different affinities, yielding very different effector functions (Bruhns et al., 2009, Blood. 113(16):3716-25, the disclosures of which are incorporated herein by reference).

Binding of IgG to the FcγRs or C1q depends on residues located in the hinge region and the CH2 domain. Two regions of the CH2 domain are critical for FcγRs and C1q binding, and have unique sequences in IgG2 and IgG4. Substitutions into human IgG1 of IgG2 residues at positions 233-236 and IgG4 residues at positions 327, 330 and 331 were shown to greatly reduce ADCC and CDC (Armour et al., 1999, Eur J immunol. 29(8):2613-24; Shields et al., 2001, J Biol Chem. 276(9):6591-604, the disclosures of which are incorporated herein by reference). Furthermore, Idusogie et al. demonstrated that alanine substitution at different positions, including K322, significantly reduced complement activation (Idusogie et al., 2000, J Immunol. 164(8):4178-84, the disclosures of which are incorporated herein by reference). Similarly, mutations in the CH2 domain of murine IgG2A were shown to reduce the binding to FcγRI, and C1q (Steurer. et al., 1995. J Immunol. 155(3):1165-74, the disclosures of which are incorporated herein by reference).

Numerous mutations have been made in the CH2 domain of human IgG1 and their effect on ADCC and CDC tested in vitro (see references cited above). Notably, alanine substitution at position 333 was reported to increase both ADCC and CDC (Shields et al., 2001, supra; Steurer et al., 1995, supra). Lazar et al. described a triple mutant (S239D/I332E/A330L) with a higher affinity for FcγRIIIa and a lower affinity for FcγRIIb resulting in enhanced ADCC (Lazar et al., 2006, PNAS 103(11):4005-4010, the disclosures of which are incorporated herein by reference). The same mutations were used to generate an antibody with increased ADCC (Ryan et al., 2007, Mol. Cancer Ther. 6:3009-3018, the disclosures of which are incorporated herein by reference). Richards et al. studied a slightly different triple mutant (S239D/I332E/G236A) with improved FcγRIIIa affinity and FcγRIIa/FcγRIIb ratio that mediates enhanced phagocytosis of target cells by macrophages (Richards et al., 2008. Mol Cancer Ther. 7(8):2517-27, the disclosures of which are incorporated herein by reference).

Due to their lack of effector functions, IgG4 antibodies represent a preferred IgG subclass for receptor modulation without cell depletion. IgG4 molecules can exchange half-molecules in a dynamic process termed Fab-arm exchange. This phenomenon can also occur in vivo between therapeutic antibodies and endogenous IgG4.

The S228P mutation has been shown to prevent this recombination process allowing the design of less unpredictable therapeutic IgG4 antibodies (Labrijn et al., 2009, Nat Biotechnol. 27(8):767-71, the disclosures of which are incorporated herein by reference).

Examples of engineered Fc regions are shown in Table I below.

TABLE I

| | | | FcR/C1q | Effector |
|---|---|---|---|---|
| Isotype | Species | Mutations* | Binding | Function |
| IgG1 | Human | T250Q/M428L¹ | Increased | Increased |
| | | | binding to FcRn | half-life |
| IgG1 | Human | M252Y/S254T/T256E + | Increased | Increased |
| | | H433K/N434F² | binding to FcRn | half-life |
| IgG1 | Human | M428L/N434S³ | Increased | Increased |
| | | | binding to FcRn | half-life |
| IgG1 | Human | E233P/L234V/L235A/?G236 + | Reduced | Reduced |
| | | A327G/A330S/P331S^{4,5} | binding to | ADCC and |
| | | | FcγRI | CDC |
| IgG1 | Human | S239D/S298A/I332E + | Increased | Increased |
| | | S239D/A330L/I332E⁶ | binding to | ADCC |
| | | | FcγRIIIa | |
| IgG1 | Human | S239D/I332E⁷ | Increased | Increased |
| | | | binding to | ADCC |
| | | | FcγRIIIa | |
| IgG1 | Human | S298A/E333A/K334A⁸ | Increased | Increased |
| | | | binding to | ADCC |
| | | | FcγRIIIa | |
| IgG1 | Human | E333A⁹ | Increased | Increased |
| | | | binding to | ADCC and |
| | | | FcγRIIIa | CDC |
| IgG1 | Human | P257I/Q311¹⁰ | Increased | Unchanged |
| | | | binding to FcRn | half-life |
| IgG1 | Human | K326W/E333S¹¹ | Increased | Increased |
| | | | binding to C1q | CDC |
| IgG1 | Human | S239D/I332E/G236A¹² | Increased | Increased |
| | | | FcγRIIa/FcγRIIb | macrophage |
| | | | ratio | phagocytosis |
| IgG1 | Human | K322A⁸ | Reduced | Reduced |
| | | | binding to C1q | CDC |
| | | N297S | | Reduced |
| | | | | (abrogated) |
| | | | | ADCC |
| | | N297Q | | Reduced |
| | | | | (abrogated) |
| | | | | ADCC |
| | | R292P + V305I +/− F243L¹³ | | Increased |
| | | | | ADCC |
| | | P247I/A339Q¹⁴ | | Increased |
| | | | | ADCC |
| IgG4 | Human | S228P¹⁵ | — | Reduced |
| | | | | Fab-arm |
| | | | | exchange |
| IgG2a | Mouse | L235E + | Reduced | Reduced |
| | | E318A/K320A/K322A¹¹ | binding to | ADCC and |
| | | | FcγRI and C1q | CDC |

*The position of the Fc amino acid mutations is defined using the Eu Numbering Scheme, which differs from the numbering in SEQ ID NOS: 18 and 19 above; see Edelman et al., 1969, Proc. Natl. Acad. Sci. USA, 63: 78-85)

### REFERENCES TO TABLE I

1. Hinton et al 2004 J. Biol. Chem. 279(8):6213-6)
2. Vaccaro et al. 2005 Nat Biotechnol. 23(10):1283-8)
3. Zalevsky et al 2010 Nat. Biotechnology 28(2):157-159
4. Armour K L. et al., 1999. Eur J Immunol. 29(8):2613-24
5. Shields R L. et al., 2001. J Biol Chem. 276(9):6591-604
6. Masuda et al. 2007, Mol Immunol. 44(12):3122-31
7. Bushfield et al 2014, Leukemia 28(11):2213-21
8. Okazaki et al. 2004, J Mol Biol.; 336(5):1239-49
9. Idusogie et al., 2000. J Immunol. 164(8):4178-84
10. Datta-Mannan A. et al., 2007. Drug Metab. Dispos. 35: 86-94
11. Steurer W. et al., 1995. J Immunol. 155(3):1165-74
12. Richards et al. 2008 Mol Cancer There. 7(8):2517-27
13. U.S. Pat. No. 7,960,512 B2
14. EP 2 213 683
15. Labrijn A F. et al., 2009. Nat Biotechnol. 27(8):767-71

In a further embodiment, the effector function of the Fc region may be altered through modification of the carbohydrate moieties within the CH2 domain therein, for example by modifying the relative levels of fucose, galactose, bisecting N-acetylglucosamine and/or sialic acid during production (see Jefferis, 2009, Nat Rev Drug Discov. 8(3):226-34 and Raju, 2008, Curr Opin Immunol., 20(4):471-8; the disclosures of which are incorporated herein by reference)

Thus, it is known that therapeutic antibodies lacking or low in fucose residues in the Fc region may exhibit enhanced ADCC activity in humans (for example, see Peipp et al., 2008, Blood 112(6):2390-9, Yamane-Ohnuki & Satoh, 2009, MAbs 1(3):230-26, lida et al., 2009, BMC Cancer 9; 58 (the disclosures of which are incorporated herein by reference). Low fucose antibody polypeptides may be produced by expression in cells cultured in a medium containing an inhibitor of mannosidase, such as kinfunensine. Low fucose antibody polypeptides exhibit increased binding to Fc receptors, including FcγRs such as FcγRIIIA.

Other methods to modify glycosylation of an antibody into a low fucose format include the use of the bacterial enzyme GDP-6-deoxy-D-Iyxo-4-hexulose reductase in cells for conversion of GDP-mannose (GDP-4-keto-6-deoxy-D-mannose) to GDP-rhamnose instead of GDP-fucose (e.g. using the GlymaxX® technology of ProBioGen AG, Berlin, Germany).

Another method to create low fucose antibodies is by inhibition or depletion of alpha-(1,6)-fucosyltransferase in the antibody-producing cells (e.g. using the Potelligent® CHOK1SV technology of Lonza Ltd, Basel, Switzerland and BioWa, Princeton, N.J., USA).

Thus, in one embodiment, the polypeptide of the invention has an Fc region with decreased fucose compared to a native human antibody.

In one embodiment, the polypeptide of the invention has an Fc region which is afucosylated (or defucosylated).

By “afucosylated”, “defucosylated” or “non-focusylated” antibodies we mean that the Fc region of the antibody does not have any fucose sugar units attached, or has a decreased content of fucose sugar units. Decreased content may be defined by the relative amount of fucose on the modified antibody compared to the fucosylated ‘wild type’ antibody, e.g. fewer fucose sugar units per immunoglobulin molecule compared to the equivalent antibody expressed in the absence of an inhibitor of mannosidase and/or in the presence of GDP-6-deoxy-D-Iyxo-4-hexulose reductase.

An exemplary heavy chain constant region amino acid sequence which may be combined with any VH region sequence disclosed herein (to form a complete heavy chain) is the IgG1 heavy chain constant region sequence reproduced here:

(SEQ ID NO: 97)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

Other heavy chain constant region sequences are known in the art and could also be combined with any VH region disclosed herein. For example, a preferred constant region is a modified IgG4 constant region such as that reproduced here:

(SEQ ID NO: 99)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVICVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNRYTQKSLSLSLGK

This modified IgG4 sequence exhibits reduced FcRn binding and hence results in a reduced serum half-life relative to wild type IgG4. In addition, it exhibits stabilization of the core hinge of IgG4 making the IgG4 more stable, preventing Fab arm exchange.

Another preferred constant region is a modified IgG4 constant region such as that reproduced here:

(SEQ ID NO: 101)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

This modified IgG4 sequence results in stabilization of the core hinge of IgG4 making the IgG4 more stable, preventing Fab arm exchange.

Also preferred is a wild type IgG4 constant region such as that reproduced here:

(SEQ ID NO: 100)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

An exemplary light chain constant region amino acid sequence which may be combined with any VL region sequence disclosed herein (to form a complete light chain) is the kappa chain constant region sequence reproduced here:

(SEQ ID NO: 98)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

Other light chain constant region sequences are known in the art and could also be combined with any VL region disclosed herein.

The antibody, or antigen binding fragment thereof, has certain preferred binding characteristics and functional effects, which are explained in more detail below. Said antibody, or antigen binding fragment thereof, preferably retains these binding characteristics and functional effects when incorporated as part of a bispecific polypeptide of the invention.

In one embodiment, the antigen-binding fragment may be selected from the group consisting of: an Fv fragment (such as a single chain Fv fragment, or a disulphide-bonded Fv fragment), a Fab-like fragment (such as a Fab fragment; a Fab′ fragment or a F(ab)₂ fragment) and domain antibodies.

In one embodiment, the bispecific polypeptide may be an IgG1 antibody with a non-immunoglobulin polypeptide (such as a CTLA-4 binding domain, e.g. CD86 or a mutated form thereof such as SEQ ID NO: 17; see below) fused to the C-terminal part of the kappa chain.

In one embodiment, the multispecific (e.g. bispecific) polypeptide may be an IgG1 antibody with a scFv fragment fused to the C-terminal end of the heavy gamma 1 chain.

In one embodiment, the multispecific (e.g. bispecific) polypeptide may contain 2-4 scFv binding to the two different targets (in this instance, GITR and CTLA-4).

By targets we include polypeptide receptors located in the cell membrane of CD3+ T cells in an activated or inactive state. Such membrane-bound receptors may be exposed extracellularly in order that they accessed by the bispecific polypeptides of the invention following administration.

It will be appreciated by persons skilled in the art that the targets (GITR and CTLA-4) may be localised on the surface of a cell. By “localised on the surface of a cell” it is meant that the target is associated with the cell such that one or more region of the target is present on the outer face of the cell surface. For example, the target may be inserted into the cell plasma membrane (i.e. orientated as a transmembrane protein) with one or more regions presented on the extracellular surface. This may occur in the course of expression of the target by the cell. Thus, in one embodiment, “localised on the surface of a cell” may mean “expressed on the surface of a cell.” Alternatively, the target may be outside the cell with covalent and/or ionic interactions localising it to a specific region or regions of the cell surface.

It will be appreciated by persons skilled in the art that the multispecific (e.g. bispecific) antibodies of the invention may be capable of inducing ADCC, ADCP, CDC and/or apoptosis.

In one embodiment of the invention, the polypeptide is capable of both targeting GITR expressing tumour cells and activating the immune system.

For example, the polypeptide may be capable of killing GITR expressing tumour cells, optionally via ADCC.

It will be appreciated that the activation of the immune system may comprise activation of effector T cells.

In a further embodiment, the polypeptide is capable of inducing tumour immunity. This can be tested in vitro in T cell activation assays, e.g. by measuring. IL-2 and IFNγ production. Activation of effector T cells would imply that a tumour specific T cell response can be achieved in vivo. Further, an anti-tumour response in an in vivo model, such as a mouse model would imply that a successful immune response towards the tumour has been achieved.

The multispecific (e.g. bispecific) antibody may modulate the activity of a cell expressing the T cell target, wherein said modulation is an increase or decrease in the activity of said cell. The cell is typically a T cell. The antibody may increase the activity of a CD4+ or CD8+ effector cell, or may decrease the activity of a regulatory T cell (Treg). In either case, the net effect of the antibody will be an increase in the activity of effector T cells. Methods for determining a change in the activity of effector T cells are well known and include, for example, measuring for an increase in the level of T cell IFNγ or IL-2 production or an increase in T cell proliferation in the presence of the antibody relative to the level of T cell IFNγ or IL-2 production and/or T cell proliferation in the presence of a control. Assays for cell proliferation and/or IFNγ or IL-2 production are well known and are exemplified in the Examples.

Standard assays to evaluate the binding ability of ligands towards targets are well known in the art, including for example, ELISAs, Western blots, RIAs, and flow cytometry analysis. The binding kinetics (e.g., binding affinity) of the polypeptide also can be assessed by standard assays known in the art, such as by Surface Plasmon Resonance analysis (SPR) or BioLayer Interferometry (BLI).

The terms “binding activity” and “binding affinity” are intended to refer to the tendency of a polypeptide molecule to bind or not to bind to a target. Binding affinity may be quantified by determining the dissociation constant (Kd) for a polypeptide and its target. A lower Kd is indicative of a higher affinity for a target. Similarly, the specificity of binding of a polypeptide to its target may be defined in terms of the comparative dissociation constants (Kd) of the polypeptide for its target as compared to the dissociation constant with respect to the polypeptide and another, non-target molecule.

The value of this dissociation constant can be determined directly by well-known methods, and can be computed even for complex mixtures by methods such as those, for example, set forth in Caceci et al. (Byte 9:340-362, 1984; the disclosures of which are incorporated herein by reference). For example, the Kd may be established using a double-filter nitrocellulose filter binding assay such as that disclosed by Wong & Lohman (Proc. Natl. Acad. Sci. USA 90, 5428-5432, 1993). Other standard assays to evaluate the binding ability of ligands such as antibodies towards targets are known in the art, including for example, ELISAs, Western blots, RIAs, and flow cytometry analysis. The binding kinetics (e.g., binding affinity) of the antibody also can be assessed by standard assays known in the art, such as by Biacore™ or Octet™ system analysis.

A competitive binding assay can be conducted in which the binding of the antibody to the target is compared to the binding of the target by another, known ligand of that target, such as another antibody. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

Alternative measures of binding affinity include EC50 or IC50. In this context EC50 indicates the concentration at which a polypeptide achieves 50% of its maximum binding to a fixed quantity of target. IC50 indicates the concentration at which a polypeptide inhibits 50% of the maximum binding of a fixed quantity of competitor to a fixed quantity of target. In both cases, a lower level of EC50 or IC50 indicates a higher affinity for a target. The EC50 and IC50 values of a ligand for its target can both be determined by well-known methods, for example ELISA. Suitable assays to assess the EC50 and IC50 are known in the art.

A multispecific (e.g. bispecific) polypeptide of the invention is preferably capable of binding to each of its targets with an affinity that is at least two-fold, 10-fold, 50-fold, 100-fold or greater than its affinity for binding to another non-target molecule.

The multispecific (e.g. bispecific) polypeptide of the invention may be produced by any suitable means. For example, all or part of the polypeptide may be expressed as a fusion protein by a cell comprising a nucleotide which encodes said polypeptide.

Alternatively, parts B1 and B2 may be produced separately and then subsequently joined together. Joining may be achieved by any suitable means, for example using the chemical conjugation methods and linkers outlined above. Separate production of parts B1 and B2 may be achieved by any suitable means. For example, by expression from separate nucleotides optionally in separate cells, as is explained in more detail below.

It will be appreciated by persons skilled in the art that the multispecific antibodies of the invention may bind to target antigens in addition to GITR and CTLA-4; in other words, the invention encompasses multispecific antibodies binding three or more targets.

For example, the multispecific polypeptide may be a trispecific antibody capable of binding GITR, CTLA-4 and a further target antigen. Thus, the further target antigen may be a further T cell target

In one embodiment, the further T cell target is a checkpoint molecule, such as a co-stimulatory or co-inhibitory molecule. By “co-stimulatory” we include co-signalling molecules which are capable of promoting T cell activation. By “co-inhibitory” we include co-signalling molecules which are capable of supressing T cell activation.

Accordingly, the further T cell target may be a stimulatory checkpoint molecule (such as CD27, CD137, CD28, ICOS and OX40). Advantageously, the multispecific polypeptide of the invention is an agonist at a stimulatory checkpoint molecule.

Alternatively, or additionally, the further T cell target may be an inhibitory checkpoint molecule (such as PD-1, Tim3, Lag3, Tigit or VISTA). Advantageously, the multispecific polypeptide of the invention is an antagonist at an inhibitory checkpoint molecule.

In one embodiment, the further T cell target is a TNFR (tumour necrosis factor receptor) superfamily member. By TNFR superfamily member we include cytokine receptors characterised by the ability to bind tumour necrosis factors (TNFs) via an extracellular cysteine-rich domain. Examples of TNFRs include OX40 and CD137.

In a further embodiment, the further T cell target may be selected from the group consisting of: OX40, CTLA-4, CD137, CD40 and CD28. For example, the first and/or second T cell target may be selected from the group consisting of OX40, CTLA-4 and CD137.

Thus, the polypeptide may be a trispecific antibody capable of binding GITR, CTLA-4 and OX40.

### Variants

The multispecific (e.g. bispecific) polypeptides or constituent binding domains thereof (such as the GITR and CTLA-4 binding domains) described herein may comprise a variant or a fragment of any of the specific amino acid sequences recited herein, provided that the polypeptide or binding domain retains binding to its target. In one embodiment, the variant of an antibody or antigen binding fragment may retain the CDR sequences of the sequences recited herein. For example, the anti-GITR antibody may comprise a variant or a fragment of any of the specific amino acid sequences recited in Table C, provided that the antibody retains binding to its target. Such a variant or fragment may typically retain the CDR sequences of the said sequence of Table C. The CTLA-4 binding domain may comprise a variant of any of the sequences of Table A, providing that that the binding domain retains binding to its target.

A fragment of any one of the heavy or light chain amino acid sequences recited herein may comprise at least 7, at least 8, at least 9, at least 10, at least 12, at least 15, at least 18, at least 20, at least 25, at least 50, at least 60, at least 70, at least 80, at least 90 or at least 100 consecutive amino acids from the said amino acid sequence.

A variant of any one of the heavy or light chain amino acid sequences recited herein may be a substitution, deletion or addition variant of said sequence. A variant may comprise 1, 2, 3, 4, 5, up to 10, up to 20, up to 30 or more amino acid substitutions and/or deletions from the said sequence. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | |
|---|---|
| Ala, A | aliphatic, hydrophobic, neutral |
| Cys, C | polar, hydrophobic, neutral |
| Asp, D | polar, hydrophilic, charged (−) |
| Glu, E | polar, hydrophilic, charged (−) |
| Phe, F | aromatic, hydrophobic, neutral |
| Gly, G | aliphatic, neutral |
| His, H | aromatic, polar, hydrophilic, |
| | charged (+) |
| Ile, I | aliphatic, hydrophobic, neutral |
| Lys, K | polar, hydrophilic, charged(+) |
| Leu, L | aliphatic, hydrophobic, neutral |
| Met, M | hydrophobic, neutral |
| Asn, N | polar, hydrophilic, neutral |
| Pro, P | hydrophobic, neutral |
| Gln, Q | polar, hydrophilic, neutral |
| Arg, R | polar, hydrophilic, charged (+) |
| Ser, S | polar, hydrophilic, neutral |
| Thr, T | polar, hydrophilic, neutral |
| Val, V | aliphatic, hydrophobic, neutral |
| Trp, W | aromatic, hydrophobic, neutral |
| Tyr, Y | aromatic, polar, hydrophobic |

Amino acids herein may be referred to by full name, three letter code or single letter code.

Preferred “derivatives” or “variants” include those in which instead of the naturally occurring amino acid the amino acid which appears in the sequence is a structural analogue thereof. Amino acids used in the sequences may also be derivatised or modified, e.g. labelled, providing the function of the antibody is not significantly adversely affected.

Derivatives and variants as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

Preferably variants have an amino acid sequence which has more than 60%, or more than 70%, e.g. 75 or 80%, preferably more than 85%, e.g. more than 90 or 95% amino acid identity to a sequence as shown in the sequences disclosed herein. This level of amino acid identity may be seen across the full length of the relevant SEQ ID NO sequence or over a part of the sequence, such as across 20, 30, 50, 75, 100, 150, 200 or more amino acids, depending on the size of the full-length polypeptide.

In connection with amino acid sequences, “sequence identity” refers to sequences which have the stated value when assessed using ClustalW (Thompson et al., 1994, Nucleic Acids Res. 22(22):4673-80; the disclosures of which are incorporated herein by reference) with the following parameters:

Pairwise alignment parameters—Method: accurate, Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatised.

### Polynucleotides, Vectors and Cells

The invention also relates to polynucleotides that encode all or part of a polypeptide of the invention. Thus, a polynucleotide of the invention may encode any polypeptide as described herein, or all or part of B1 or all or part of B2. The terms “nucleic acid molecule” and “polynucleotide” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated.

A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Representative polynucleotides which encode examples of a heavy chain or light chain amino acid sequence of an antibody may comprise or consist of any one of the nucleotide sequences disclosed herein, for example the sequences set out in Table C. Representative polynucleotides which encode the polypeptides shown in Table C may comprise or consist of the corresponding nucleotide sequences which are also shown in Table C (intron sequences are shown in lower case). Representative polynucleotides which encode examples of CTLA-4 binding domains may comprise or consist of any one of SEQ ID NOS: 25 to 43 as shown in Table B.

A suitable polynucleotide sequence may alternatively be a variant of one of these specific polynucleotide sequences. For example, a variant may be a substitution, deletion or addition variant of any of the above nucleic acid sequences. A variant polynucleotide may comprise 1, 2, 3, 4, 5, up to 10, up to 20, up to 30, up to 40, up to 50, up to 75 or more nucleic acid substitutions and/or deletions from the sequences given in the sequence listing.

Suitable variants may be at least 70% homologous to a polynucleotide of any one of nucleic acid sequences disclosed herein, preferably at least 80 or 90% and more preferably at least 95%, 97% or 99% homologous thereto. Preferably homology and identity at these levels is present at least with respect to the coding regions of the polynucleotides. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may exist over the entire length of the unmodified polynucleotide sequence.

Methods of measuring polynucleotide homology or identity are known in the art. For example, the UWGCG Package provides the BESTFIT program which can be used to calculate homology (e.g. used on its default settings) (Devereux et al, 1984, Nucleic Acids Research 12:387-395; the disclosures of which are incorporated herein by reference).

The PILEUP and BLAST algorithms can also be used to calculate homology or line up sequences (typically on their default settings), for example as described in Altschul, 1993, J Mol Evol 36:290-300; Altschul et al, 1990, J Mol Biol 215:403-10, the disclosures of which are incorporated herein by reference).

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff & Henikoff, 1992, Proc. Natl. Acad. Sci. USA 89:10915-10919; the disclosures of which are incorporated herein by reference) alignments (B) of 50, expectation (E) of 10, M=5, N=4, and a comparison of both strands.

The BLAST algorithm performs a statistical analysis of the similarity between two sequences; see e.g. Karlin & Altschul, 1993, Proc. Natl. Acad. Sci. USA 90:5873-5787; the disclosures of which are incorporated herein by reference. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologue may differ from a sequence in the relevant polynucleotide by less than 3, 5, 10, 15, 20 or more mutations (each of which may be a substitution, deletion or insertion). These mutations may be measured over a region of at least 30, for instance at least 40, 60 or 100 or more contiguous nucleotides of the homologue.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

A polypeptide of the invention may thus be produced from or delivered in the form of a polynucleotide which encodes and is capable of expressing it.

Polynucleotides of the invention can be synthesised according to methods well known in the art, as described by way of example in Green & Sambrook (2012, Molecular Cloning—a laboratory manual, 4^{th} edition; Cold Spring Harbor Press; the disclosures of which are incorporated herein by reference).

The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art (see Green & Sambrook, supra).

The invention also includes cells that have been modified to express a polypeptide of the invention. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors or expression cassettes encoding for a polypeptide of the invention include mammalian HEK293T, CHO, HeLa, NSO and COS cells. Preferably the cell line selected will be one which is not only stable, but also allows for mature glycosylation and cell surface expression of a polypeptide.

Such cell lines of the invention may be cultured using routine methods to produce a polypeptide of the invention, or may be used therapeutically or prophylactically to deliver antibodies of the invention to a subject. Alternatively, polynucleotides, expression cassettes or vectors of the invention may be administered to a cell from a subject ex vivo and the cell then returned to the body of the subject.

### Pharmaceutical Formulations, Therapeutic Uses and Patient Groups

In another aspect, the present invention provides compositions comprising molecules of the invention, such as the antibodies, multispecific (e.g. bispecific) polypeptides, polynucleotides, vectors and cells described herein. For example, the invention provides a composition comprising one or more molecules of the invention, such as one or more antibodies and/or bispecific polypeptides of the invention, and at least one pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral, e.g. intravenous, intramuscular or subcutaneous administration (e.g., by injection or infusion). Depending on the route of administration, the polypeptide may be coated in a material to protect the polypeptide from the action of acids and other natural conditions that may inactivate or denature the polypeptide.

Preferred pharmaceutically acceptable carriers comprise aqueous carriers or diluents. Examples of suitable aqueous carriers that may be employed in the compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, or sodium chloride in the composition.

A composition of the invention also may include a pharmaceutically acceptable anti-oxidant. These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminium monostearate and gelatin.

Therapeutic compositions typically must be sterile and stable under the conditions of manufacture and storage. The composition can be formulated as a solution, microemulsion, liposome, or other ordered structure suitable to high drug concentration.

Sterile injectable solutions can be prepared by incorporating the active agent (e.g. polypeptide) in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof.

Particularly preferred compositions are formulated for systemic administration or for local administration. Local administration may be at the site of a tumour or into a tumour draining lymph node. The composition may preferably be formulated for sustained release over a period of time. Thus, the composition may be provided in or as part of a matrix facilitating sustained release. Preferred sustained release matrices may comprise a montanide or γ-polyglutamic acid (PGA) nanoparticles. Localised release of a polypeptide of the invention, optionally over a sustained period of time, may reduce potential autoimmune side-effects associated with administration of a CTLA-4 antagonist.

Compositions of the invention may comprise additional active ingredients as well as a polypeptide of the invention. As mentioned above, compositions of the invention may comprise one or more polypeptides of the invention. They may also comprise additional therapeutic or prophylactic agents.

Also within the scope of the present invention are kits comprising polypeptides or other compositions of the invention and instructions for use. The kit may further contain one or more additional reagents, such as an additional therapeutic or prophylactic agent as discussed above.

The polypeptides in accordance with the present invention maybe used in therapy or prophylaxis. In therapeutic applications, polypeptides or compositions are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. In prophylactic applications, polypeptides or compositions are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means.

In particular, antibodies and bispecific polypeptides of the invention may be useful in the treatment or prevention of cancer. Accordingly, the invention provides an antibody or bispecific polypeptide of the invention for use in the treatment or prevention of cancer. The invention also provides a method of treating or preventing cancer comprising administering to an individual a polypeptide of the invention. The invention also provides an antibody or bispecific polypeptide of the invention for use in the manufacture of a medicament for the treatment or prevention of cancer.

The cancer may be prostate cancer, breast cancer, colorectal cancer, pancreatic cancer, ovarian cancer, lung cancer, cervical cancer, rhabdomyosarcoma, neuroblastoma, multiple myeloma, leukemia, acute lymphoblastic leukemia, melanoma, bladder cancer, gastric cancer, head and neck cancer, liver cancer, skin cancer, lymphoma or glioblastoma.

An antibody or bispecific polypeptide of the present invention, or a composition comprising said antibody or said polypeptide, may be administered via one or more routes of administration using one or more of a variety of methods known in the art. As will be appreciated by the skilled artisan, the route and/or mode of administration will vary depending upon the desired results. Systemic administration or local administration are preferred. Local administration may be at the site of a tumour or into a tumour draining lymph node. Preferred modes of administration for polypeptides or compositions of the invention include intravenous, intramuscular, intradermal, intraperitoneal, subcutaneous, spinal or other parenteral modes of administration, for example by injection or infusion. The phrase “parenteral administration” as used herein means modes of administration other than enteral and topical administration, usually by injection. Alternatively, a polypeptide or composition of the invention can be administered via a non-parenteral mode, such as a topical, epidermal or mucosal mode of administration.

A suitable dosage of an antibody or polypeptide of the invention may be determined by a skilled medical practitioner. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular polypeptide employed, the route of administration, the time of administration, the rate of excretion of the polypeptide, the duration of the treatment, other drugs, compounds and/or materials used in combination with the particular compositions employed, the age, sex, weight, condition, general health and prior medical history of the patient being treated, and like factors well known in the medical arts.

A suitable dose of an antibody or polypeptide of the invention may be, for example, in the range of from about 0.1 μg/kg to about 100 mg/kg body weight of the patient to be treated. For example, a suitable dosage may be from about 1 μg/kg to about 10 mg/kg body weight per day or from about 10 g/kg to about 5 mg/kg body weight per day.

Dosage regimens may be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Antibodies or polypeptides may be administered in a single dose or in multiple doses. The multiple doses may be administered via the same or different routes and to the same or different locations. Alternatively, antibodies or polypeptides can be administered as a sustained release formulation as described above, in which case less frequent administration is required. Dosage and frequency may vary depending on the half-life of the polypeptide in the patient and the duration of treatment that is desired. The dosage and frequency of administration can also vary depending on whether the treatment is prophylactic or therapeutic. In prophylactic applications, a relatively low dosage may be administered at relatively infrequent intervals over a long period of time. In therapeutic applications, a relatively high dosage may be administered, for example until the patient shows partial or complete amelioration of symptoms of disease.

Combined administration of two or more agents may be achieved in a number of different ways. In one embodiment, the antibody or polypeptide and the other agent may be administered together in a single composition. In another embodiment, the antibody or polypeptide and the other agent may be administered in separate compositions as part of a combined therapy. For example, the modulator may be administered before, after or concurrently with the other agent.

An antibody, polypeptide or composition of the invention may also be used in a method of increasing the activation of a population of cells expressing GITR and CTLA-4, the method comprising administering to said population of cells a polypeptide or composition of the invention under conditions suitable to permit interaction between said cell and a polypeptide of the invention. The population of cells typically comprises at least some cells which express GITR, typically T cells, and at least some cells which express CTLA-4. The method is typically carried out ex vivo.

### Binding Domains for GITR

The bispecific polypeptides of the invention comprise a binding domain which is specific for glucocorticoid-induced TNFR-related protein (GITR; also known as tumour necrosis factor receptor superfamily member 18 [TNFRSF18] and activation-inducible TNFR family receptor [AITR]).

The antibody, or antigen binding fragment thereof, that binds specifically to GITR has certain preferred binding characteristics and functional effects, which are explained in more detail below. Said antibody, or antigen binding fragment thereof, preferably retains these binding characteristics and functional effects when incorporated as part of a bispecific antibody of the invention. The invention also provides said antibody as an antibody or antigen-binding fragment thereof in isolated form, i.e. independently of a bispecific antibody of the invention.

The anti-GITR domain (B1) preferably specifically binds to GITR, i.e. it binds to GITR but does not bind, or binds at a lower affinity, to other molecules. The term “GITR” as used herein typically refers to human GITR. The amino acid sequence of human GITR is set out in SEQ ID NO: 111 (corresponding to GenBank: AAI52382.1). The B1 domain may have some binding affinity for GITR from other mammals, such as GITR from a non-human primate, for example Macaca fascicularis (cynomolgus monkey). The B1 domain preferably does not bind to murine GITR and/or does not bind to other human TNFR superfamily members, for example human CD137, OX40 or CD40.

The B1 domain has the ability to bind to GITR in its native state and in particular to GITR localised on the surface of a cell. “Localised on the surface of a cell” is as defined previously. Preferably, the B1 domain will bind specifically to GITR. That is, the B1 domain will preferably bind to GITR with greater binding affinity than that at which it binds to another molecule.

Preferably, the above binding properties of the B1 domain are substantially maintained in the bispecific antibody of the invention.

Thus, the bispecific antibody may modulate the activity of a cell expressing GITR, wherein said modulation is an increase or decrease in the activity of said cell. The cell is typically a T cell. The antibody may increase the activity of a CD4+ or CD8+ effector T cell, or may decrease the activity of, or deplete, a Treg cell. In either case, the net effect of the antibody will be an increase in the activity of Teff cells, particularly CD4+, CD8+ or NK effector T cells. Methods for determining a change in the activity of effector T cells are well known and are as described earlier.

The antibody preferably causes an increase in activity in a CD8+ T cell in vitro, optionally wherein said increase in activity is an increase in proliferation, IFN-γ production and/or IL-2 production by the T cell. The increase is preferably at least 2-fold, more preferably at least 10-fold and even more preferably at least 25-fold higher than the change in activity caused by an isotype control antibody measured in the same assay.

The antibody preferably binds to human GITR with a Kd value which is less than 10×10⁻⁹M or less than 7×10⁻⁹M, more preferably less than 4, or 2×10⁻⁹M, most preferably less than 1×10⁻⁹M.

For example, the antibody preferably does not bind to murine GITR or any other TNFR superfamily member, such as OX40 or CD40. Therefore, typically, the Kd for the antibody with respect to human GITR will be 2-fold, preferably 5-fold, more preferably 10-fold less than Kd with respect to the other, non-target molecule, such as murine GITR, other TNFR superfamily members, or any other unrelated material or accompanying material in the environment. More preferably, the Kd will be 50-fold less, even more preferably 100-fold less, and yet more preferably 200-fold less.

The value of this dissociation constant can be determined directly by well-known methods, as described earlier. A competitive binding assay can also be conducted, as described earlier.

An antibody of the invention is preferably capable of binding to its target with an affinity that is at least two-fold, 10-fold, 50-fold, 100-fold or greater than its affinity for binding to another non-target molecule.

In summary therefore, the anti-GITR antibody preferably exhibits at least one of the following functional characteristics:
I. binding to human GITR with a K_{D} value which is less than 10×10⁻⁹M, more preferably less than 1.2×10⁻⁹M; and
II. is capable of causing an increase in activity in a CD3+ T cell in vitro, optionally wherein said increase in activity is an increase in proliferation, IFN-γ production and/or IL-2 production by the T cell. The increase is preferably at least 2-fold, more preferably at least 10-fold and even more preferably at least 25-fold higher than the change in activity caused by an isotype control antibody measured in the same assay.

The antibody is specific for GITR, typically human GITR and may comprise any one, two, three, four, five or all six of the exemplary CDR sequences of any corresponding pair of rows in Tables D(1) and D(2).

For example, the antibody may comprise any one, two, three, four, five or all six of the exemplary CDR sequences of the first rows of Table D(1) and Table D(2) (SEQ ID NOs: 76, 77, 78, 88, 89, 90)

Alternatively the antibody may comprise any one, two, three, four, five or all six of the exemplary CDR sequences of the second, third or fourth rows of Tables D(1) and D(2).

Preferred anti-GITR antibodies may comprise at least a heavy chain CDR3 as defined in any individual row of Table D(1) and/or a light chain CDR3 as defined in in any individual row of Table D(2). The antibody may comprise all three heavy chain CDR sequences shown in an individual row of Table D(1) (that is, all three heavy chain CDRs of a given “VH number”) and/or all three light chain CDR sequences shown in an individual row of Table D(2) (that is, all three light chain CDRs of a given “VL number”).

Examples of complete heavy and light chain variable region amino acid sequences of anti-GITR antibodies are shown in Table C. Exemplary nucleic acid sequences encoding each amino acid sequence are also shown. SEQ ID NOs 52 to 67 refer to the relevant amino acid and nucleotide sequences of anti-GITR antibodies. The numbering of said VH and VL regions in Table C corresponds to the numbering system used as in Table D(1) and (2). Thus, for example, the amino acid sequence for “2349, light chain VL” is an example of a complete VL region sequence comprising all three CDRs of VL number 2349 shown in Table D(2) and the amino acid sequence for “2348, heavy chain VH” is an example of a complete VH region sequence comprising all three CDRs of VH number 2348 shown in Table D(1).

Preferred anti-GITR antibodies of the invention include a VH region which comprises all three CDRs of a particular VH number and a VL region which comprises all three CDRs of a particular VL number. For example: an antibody may comprise all three CDRs of VH number 2348 and all three CDRs of VL number 2349. Such an antibody may preferably comprise the corresponding complete VH and VL sequences of 2348 and 2349 (mAb—without CTLA-4 binding domain) as shown in Table C (SEQ ID NOs: 52 and 61).

An antibody may alternatively comprise all three CDRs of VH number 2372 and all three CDRs of VL number 2373. Such an antibody may preferably comprise the corresponding complete VH and VL sequences of 2372 and 2373 (mAb—without CTLA-4 binding domain) as shown in Table C (SEQ ID NOs: 54 and 63).

An antibody may alternatively comprise all three CDRs of VH number 2396 and all three CDRs of VL number 2397. Such an antibody may preferably comprise the corresponding complete VH and VL sequences of 2396 and 2397 (mAb—without CTLA-4 binding domain) as shown in Table C (SEQ ID NOs: 56 and 65).

An antibody may alternatively comprise all three CDRs of VH number 2404 and all three CDRs of VL number 2405. Such an antibody may preferably comprise the corresponding complete VH and VL sequences of 2404 and 2405 (mAb—without CTLA-4 binding domain) as shown in Table C (SEQ ID NOs: 58 and 67)

The anti-GITR antibody may bind to the same epitope as any of the specific anti-GITR antibodies described herein.

In an alternative embodiment, the binding domain (B1) may be capable of competitively inhibiting the binding to human GITR of one or more of the exemplary GITR binding domains described herein, e.g. an antibody or fragment or variant thereof comprising a light chain variable region amino acid sequence selected from the group consisting of SEQ ID NOs: 61, 63, 65 and 67 and a heavy chain variable region amino acid sequence selected from the group consisting of SEQ ID NOs: 52, 54, 56 and 58.

Competitive binding typically arises because the test antibody binds at, or at least in close proximity to, the epitope on the antigen to which binds the reference antibody (in this case, 1630/1631). However, it will be appreciated by persons skilled in the art that competitive binding may also arise by virtue of steric interference; thus, the test antibody may bind at an epitope different from that to which the reference antibody binds but may still be of sufficient size or configuration to hinder the binding of the reference antibody to the antigen.

Methods for identifying polypeptides capable competitively inhibiting the binding of a reference polypeptide to a target are well known in the art, e.g. ELISA, BLI or SPR.

### Binding Domains for CTLA-4

The multispecific (e.g. bispecific) polypeptides of the invention also comprise a binding domain specific for cytotoxic T-lymphocyte-associated protein 4 (CTLA-4; also known as CD152).

The amino acid sequence of human CTLA-4 is provided in SEQ ID NO:1.

CD86 and CD80 may be referred to herein as B7 proteins (B7-2 and B7-1 respectively). These proteins are expressed on the surface of antigen presenting cells and interact with the T cell receptors CD28 and CTLA-4. The binding of the B7 molecules to CD28 promotes T cell activation while binding of B7 molecules to CTLA-4 switches off the activation of the T cell. The interaction between the B7 proteins with CD28 and/or CTLA-4 constitutes a costimulatory signalling pathway which plays an important role in immune activation and regulation. Thus, the B7 molecules are part of a pathway, amenable to manipulation in order to uncouple immune inhibition, thereby enhancing immunity in patients.

The CD86 protein is a monomer and consists of two extracellular immunoglobulin superfamily domains. The receptor binding domain of CD86 has a typical IgV-set structure, whereas the membrane proximal domain has a C1-set like structure. The structures of CD80 and CD86 have been determined on their own or in complex with CTLA-4. The contact residues on the CD80 and CD86 molecules are in the soluble extracellular domain, and mostly located in the beta-sheets and not in the (CDR-like) loops.

SEQ ID NO: 3 is the amino acid sequence of the monomeric soluble extracellular domain of human wild-type CD86. This wild type sequence may optionally lack Alanine and Proline at the N terminus, i.e. positions 24 and 25. These amino acids may be referred to herein as A24 and P25 respectively.

A bispecific polypeptide of the invention may incorporate as a polypeptide binding domain a domain which is specific for CTLA-4, a “CTLA-4 binding domain”. Suitable examples of such binding domains are disclosed in WO 2014/207063, the contents of which are incorporated by reference. The binding domain specific for CTLA-4 may also bind to CD28. The term CTLA-4 as used herein typically refers to human CTLA-4 and the term CD28 as used herein typically refers to human CD28. The sequences of human CTLA-4 and human CD28 are set out in SEQ ID NOs: 1 and 2 respectively. The CTLA-4 binding domain of the polypeptide of the present invention may have some binding affinity for CTLA-4 or CD28 from other mammals, for example primate or murine CTLA-4 or CD28.

The CTLA-4 binding domain has the ability to bind to CTLA-4 in its native state and in particular to CTLA-4 localised on the surface of a cell. “Localised on the surface of a cell” is as defined above.

The CTLA-4 binding domain part of the polypeptide of the invention may comprise or consist of:
(i) the amino acid sequence of SEQ ID NO: 3; or
(ii) an amino acid sequence in which at least one amino acid is changed when compared to the amino acid sequence of SEQ ID NO: 3 provided that said binding domain binds to human CTLA-4 with higher affinity than wild-type human CD86.

In other words, the CTLA-4 binding domain is a polypeptide binding domain specific for human CTLA-4 which comprises or consists of (i) the monomeric soluble extracellular domain of human wild-type CD86, or (ii) a polypeptide variant of said soluble extracellular domain, provided that said polypeptide variant binds to human CTLA-4 with higher affinity than wild-type human CD86.

Accordingly, the CTLA-4 binding domain of the polypeptide of the invention may have the same target binding properties as human wild-type CD86, or may have different target binding properties compared to the target binding properties of human wild-type CD86. For the purposes of comparing such properties, “human wild-type CD86” typically refers to the monomeric soluble extracellular domain of human wild-type CD86 as described in the preceding section.

Human wild-type CD86 specifically binds to two targets, CTLA-4 and CD28. Accordingly, the binding properties of the CTLA-4 binding domain of the polypeptide of the invention may be expressed as an individual measure of the ability of the polypeptide to bind to each of these targets. For example, a polypeptide variant of the monomeric extracellular domain of human wild-type CD86 preferably binds to CTLA-4 with a higher binding affinity than that of wild-type human CD86 for CTLA-4. Such a polypeptide may optionally also bind to CD28 with a lower binding affinity than that of wild-type human CD86 for CD28.

The CTLA-4 binding domain of the polypeptide of the invention is a polypeptide binding domain specific for CTLA-4. This means that it binds to CTLA-4 preferably with a greater binding affinity than that at which it binds to another molecule. The CTLA-4 binding domain preferably binds to CTLA-4 with the same or with a higher affinity than that of wild-type human CD86 for CTLA-4.

Preferably, the Kd of the CTLA-4 binding domain of the polypeptide of the invention for human CTLA-4 will be at least 2-fold, at least 2.5-fold, at least 3-fold, at least 3.5-fold, at least 4-fold, at least 4.5-fold, at least 5-fold, at least 5.5-fold, at least 8-fold or at least 10-fold less than the Kd of wild-type human CD86 for human CTLA-4. Most preferably, the Kd of the CTLA-4 binding domain for human CTLA-4 will be at least 5-fold or at least 10-fold less than the Kd of wild-type human CD86 for human CTLA-4. A preferred method for determining the Kd of a polypeptide for CTLA-4 is SPR analysis, e.g. with a Biacore™ system. Suitable protocols for the SPR analysis of polypeptides are known in the art.

Preferably, the EC50 of the CTLA-4 binding domain of the polypeptide of the invention for human CTLA-4 will be at least 1.5-fold, at least 2-fold, at least 3-fold, at least 5-fold, at least 10-fold, at least 12-fold, at least 14-fold, at least 15-fold, at least 17-fold, at least 20-fold, at least 25-fold or at least 50-fold less than the EC50 of wild-type human CD86 for human CTLA-4 under the same conditions. Most preferably, the EC50 of the CTLA-4 binding domain for human CTLA-4 will be at least 10-fold or at least 25-fold less than the EC50 of wild-type human CD86 for human CTLA-4 under the same conditions. A preferred method for determining the EC50 of a polypeptide for CTLA-4 is via ELISA. Suitable ELISA assays for use in the assessment of the EC50 of polypeptides are known in the art.

Preferably, the IC50 of the CTLA-4 binding domain of the polypeptide of the invention when competing with wild-type human CD86 for binding to human CTLA-4 will be at least 2-fold, at least 3-fold, at least 4-fold, at least 5-fold, at least 10-fold, at least 13-fold, at least 15-fold, at least 50-fold, at least 100-fold, or at least 300-fold less than the IC50 of wild-type human CD86 under the same conditions. Most preferably, the IC50 of the CTLA-4 binding domain will be at least 10-fold or at least 300-fold less than the IC50 of wild-type human CD86 under the same conditions. A preferred method for determining the IC50 of a polypeptide of the invention is via ELISA. Suitable ELISA assays for use in the assessment of the IC50 of polypeptides of the invention are known in the art.

The CTLA-4 binding domain of the polypeptide of the invention may also bind specifically to CD28. That is, the CTLA-4 binding domain may bind to CD28 with greater binding affinity than that at which it binds to another molecule, with the exception of CTLA-4. The CTLA-4 binding domain may bind to human CD28 with a lower affinity than that of wild-type human CD86 for human CD28. Preferably, the Kd of the CTLA-4 binding domain for human CD28 will be at least 2-fold, preferably at least 5-fold, more preferably at least 10-fold higher than the Kd of wild-type human CD86 for human CD28.

The binding properties of the CTLA-4 binding domain of the polypeptide of the invention may also be expressed as a relative measure of the ability of a polypeptide to bind to the two targets, CTLA-4 and CD28. That is, the binding properties of the CTLA-4 binding domain may be expressed as a relative measure of the ability of the polypeptide to bind to CTLA-4 versus its ability to bind to CD28. Preferably the CTLA-4 binding domain has an increased relative ability to bind to CTLA-4 versus CD28, when compared to the corresponding relative ability of human wild-type CD86 to bind to CTLA-4 versus CD28.

When the binding affinity of a polypeptide for both CTLA-4 and CD28 is assessed using the same parameter (e.g. Kd, EC50), then the relative binding ability of the polypeptide for each target may be expressed as a simple ratio of the values of the parameter for each target. This ratio may be referred to as the binding ratio or binding strength ratio of a polypeptide. For many parameters used to assess binding affinity (e.g. Kd, EC50), a lower value indicates a higher affinity. When this is the case, the ratio of binding affinities for CTLA-4 versus CD28 is preferably expressed as a single numerical value calculated according to the following formula:

Binding ratio=[binding affinity for CD28]÷[binding affinity for CTLA-4]

Alternatively, if binding affinity is assessed using a parameter for which a higher value indicates a higher affinity, the inverse of the above formula is preferred. In either context, the CTLA-4 binding domain of the polypeptide of the invention preferably has a higher binding ratio than human wild-type CD86. It will be appreciated that direct comparison of the binding ratio for a given polypeptide to the binding ratio for another polypeptide typically requires that the same parameters be used to assess the binding affinities and calculate the binding ratios for both polypeptides.

Preferably, the binding ratio for a polypeptide is calculated by determining the Kd of the polypeptide for each target and then calculating the ratio in accordance with the formula [Kd for CD28]÷[Kd for CTLA-4]. This ratio may be referred to as the Kd binding ratio of a polypeptide. A preferred method for determining the Kd of a polypeptide for a target is SPR analysis, e.g. with a Biacore™ system. Suitable protocols for the SPR analysis of polypeptides of the invention are set out in the Examples. The binding ratio of the CTLA-4 binding domain of the polypeptide of the invention calculated according to this method is preferably at least 2-fold or at least 4-fold higher than the binding ratio of wild-type human CD86 calculated according to the same method.

Alternatively, the binding ratio for a polypeptide may be calculated by determining the EC50 of the polypeptide for each target and then calculating the ratio in accordance with the formula [EC50 for CD28]÷[EC50 for CTLA-4]. This ratio may be referred to as the EC50 binding ratio of a polypeptide. A preferred method for determining the EC50 of a polypeptide for a target is via ELISA. Suitable ELISA assays for use in the assessment of the EC50 of polypeptides of the invention known in the art. The binding ratio of the CTLA-4 binding domain of the polypeptide of the invention calculated according to this method is at least 2-fold, at least 3-fold, at least 4-fold, at least 5-fold, at least 6-fold, at least 7-fold, at least 8-fold, at least 9-fold or at least 10-fold higher than the binding ratio of wild-type human CD86 calculated according to the same method.

The CTLA-4 binding domain of the polypeptide of the invention may have the ability to cross-compete with another polypeptide for binding to CTLA-4. For example, the CTLA-4 binding domain may cross-compete with a polypeptide having the amino acid sequence of any one of SEQ ID NOs: 6 to 24 for binding to CTLA-4. Such cross-competing polypeptides may be identified in standard binding assays. For example, SPR analysis (e.g. with a Biacore™ system), ELISA assays or flow cytometry may be used to demonstrate cross-competition.

In addition to the above functional characteristics, the CTLA-4 binding domain of the polypeptide of the invention has certain preferred structural characteristics. The CTLA-4 binding domain either comprises or consists of (i) the monomeric soluble extracellular domain of human wild-type CD86, or (ii) a polypeptide variant of said soluble extracellular domain, provided that said polypeptide variant binds to human CTLA-4 with higher affinity than wild-type human CD86.

A polypeptide variant of the monomeric soluble extracellular domain of human wild-type CD86 comprises or consists of an amino acid sequence which is derived from that of human wild-type CD86, specifically the amino acid sequence of the soluble extracellular domain of human wild-type CD86 (SEQ ID NO: 3), optionally lacking A24 and P25. In particular, a variant comprises an amino acid sequence in which at least one amino acid is changed when compared to the amino acid sequence of SEQ ID NO: 3 (or said sequence lacking A24 and P25). By “changed” it is meant that at least one amino acids is deleted, inserted, or substituted compared to the amino acid sequence of SEQ ID NO: 3 (or said sequence lacking A24 and P25). By “deleted” it is meant that the at least one amino acid present in the amino acid sequence of SEQ ID NO: 3 (or said sequence lacking A24 and P25) is removed, such that the amino acid sequence is shortened by one amino acid. By “inserted” it is meant that the at least one additional amino acid is introduced into the amino acid sequence of SEQ ID NO: 3 (or said sequence lacking A24 and P25), such that the amino acid sequence is lengthened by one amino acid. By “substituted” it is meant that the at least one amino acid in the amino acid sequence of SEQ ID NO: 3 (or said sequence lacking A24 and P25) is replaced with an alternative amino acid.

Typically, at least 1, 2, 3, 4, 5, 6, 7, 8 or 9 amino acids are changed when compared to the amino acid sequence of SEQ ID NO: 3 (or said sequence lacking A24 and P25). Typically, no more than 10, 9, 8, 7, 6, 5, 4, 2 or 1 amino acids are changed when compared to the amino acid sequence of SEQ ID NO: 3 (or said sequence lacking A24 and P25). It will be appreciated that any of these lower limits may be combined with any of these upper limits to define a range for the permitted number of changes compared to the amino acid sequence of SEQ ID NO: 3 (or said sequence lacking A24 and P25). Thus, for example, a polypeptide of the invention may comprise an amino acid sequence in which the permitted number of amino acid changes compared to the amino acid sequence of SEQ ID NO: 3 (or said sequence lacking A24 and P25) is in the range 2 to 3, 2 to 4, 2 to 5, 2 to 6, 2 to 7, 2 to 8, 2 to 9, 2 to 10, 3 to 4, 3 to 5, 3 to 6, and so on.

It is particularly preferred that at least 2 amino acids are changed when compared to the amino acid sequence of SEQ ID NO: 3 (or said sequence lacking A24 and P25). Preferably, the permitted number of amino acid changes compared to the amino acid sequence of SEQ ID NO: 3 (or said sequence lacking A24 and P25) is in the range 2 to 9, 2 to 8 or 2 to 7.

The numbers and ranges set out above may be achieved with any combination of deletions, insertions or substitutions compared to the amino acid sequence of SEQ ID NO: 3 (or said sequence lacking A24 and P25). For example, there may be only deletions, only insertions, or only substitutions compared to the amino acid sequence of SEQ ID NO: 3 (or said sequence lacking A24 and P25), or any mixture of deletions, insertions or substitutions. Preferably the variant comprises an amino acid sequence in which all of the changes compared to the amino acid sequence of SEQ ID NO: 3 (or said sequence lacking A24 and P25) are substitutions. That is, a sequence in which no amino acids are deleted or inserted compared to the sequence of SEQ ID NO: 3 (or said sequence lacking A24 and P25). In the amino acid sequence of a preferred variant, 1, 2, 3, 4, 5, 6, 7, or 8 amino acids are substituted when compared to the amino acid sequence of SEQ ID NO: 3 (or said sequence lacking A24 and P25) and no amino acids are deleted or inserted compared to the sequence of SEQ ID NO: 3 (or said sequence lacking A24 and P25).

Preferably the changes compared to the sequence of SEQ ID NO: 3 (or said sequence lacking A24 and P25) are in the FG loop region (positions 114 to 121) and/or the beta sheet region of SEQ ID NO: 3. The strands of the beta sheet region have the following positions in SEQ ID NO: 3: A:27-31, B:36-37, C:54-58, C′:64-69, C″:72-74, D:86-88, E:95-97, F:107-113, G:122-133.

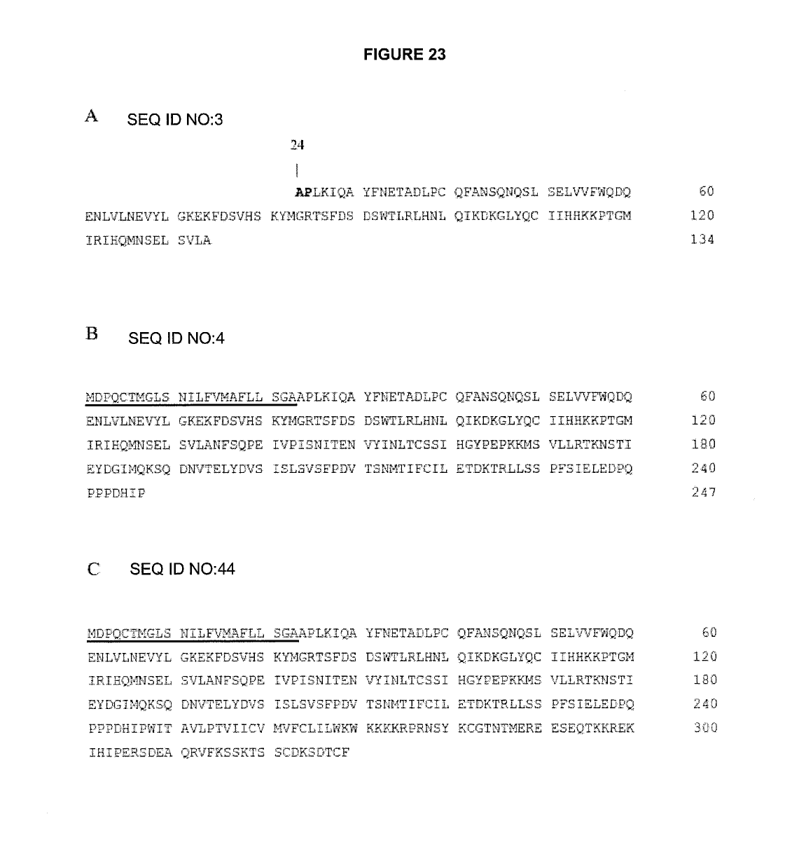

Most preferably, the changes compared to the sequence of SEQ ID NO: 3 (or said sequence lacking A24 and P25) are in one or more of the positions selected from 32, 48, 49, 54, 74, 77, 79, 103, 107, 111, 118, 120, 121, 122, 125, 127 or 134. All numbering of amino acid positions herein is based on counting the amino acids in SEQ ID NO: 4 starting from the N terminus. Thus, the first position at the N terminus of SEQ ID NO: 3 is numbered 24 (see schematic diagram in FIG. 23).

Particularly preferred insertions include a single additional amino acid inserted between positions 116 and 117 and/or a single additional amino acid inserted between positions 118 and 119. The inserted amino acid is preferably Tyrosine (Y), Serine (S), Glycine (G), Leucine (L) or Aspartic Acid (D).

A particularly preferred substitution is at position 122, which is Arginine (R). The polypeptide of the invention preferably includes an amino acid sequence in which at least position 122 is substituted compared to the amino acid sequence of SEQ ID NO: 3 (or said sequence lacking A24 and P25). The most preferred substitution at position 122 is to replace Arginine (R) with Lysine (K) or Asparagine (N), ranked in order of preference. This substitution may be referred to as R122K/N.

Other preferred substitutions are at positions 107, 121, and 125, which are Leucine (L), Isoleucine (I) and Glutamic acid (Q), respectively. In addition to the substitution at position 122, the polypeptide of the invention preferably includes an amino acid sequence in which at least one of the amino acids at positions 107, 121 and 125 is also substituted compared to the amino acid sequence of SEQ ID NO: 3 (or said sequence lacking A24 and P25). The amino acid sequence of the polypeptide of the invention may also be substituted at one or more of positions 32, 48, 49, 54, 64, 74, 77, 79, 103, 111, 118, 120, 127 and 134.

The most preferred substitution at position 107 is to replace Leucine (L) with Isoleucine (I), Phenylalanine (F) or Arginine (R), ranked in order of preference. This substitution may be referred to as L107I/F/R. Similar notation is used for other substitutions described herein. The most preferred substitution at position 121 is to replace Isoleucine (I) with Valine (V). This substitution may be referred to as I121V.

The most preferred substitution at position 125 is to replace Glutamine (Q) with Glutamic acid (E). This substitution may be referred to as Q125E.

Other substitutions which may be preferred in the amino acid sequence of the polypeptide of the invention include: F32I, Q48L, S49T, V54I, V64I, K74I/R, S77A, H79D/S/A, K103E, I111V, T118S, M120L, N127S/D and A134T.

Particularly preferred variants of said soluble extracellular domain of human wild-type CD86 comprise or consist of any one of the amino acid sequences of SEQ ID NOs: 6 to 24, as shown in Table A.

The amino acid sequences shown in SEQ ID NOs: 6 to 14 may optionally include the additional residues AP at the N-terminus. The amino acid sequences shown in SEQ ID NOs: 15 to 24 may optionally lack the residues AP at the N-terminus. In either case, these residues correspond to A24 and P25 of SEQ ID NO: 3.

The CTLA-4 binding domain of the polypeptide of the invention may comprise or consist of any of the above-described variants of said soluble extracellular domain of human wild-type CD86. That is, the CTLA-4 binding domain of the polypeptide of the invention may comprise or consist of the amino acid sequence of any one of SEQ ID NOs: 6 to 24, as shown in Table A.

The binding domain may modulate signalling from CTLA-4, for example when administered to a cell expressing CTLA-4, such as a T cell. Preferably the binding domain reduces, i.e. inhibits or blocks, said signalling and thereby increases the activation of said cell. Changes in CTLA-4 signalling and cell activation as a result of administration of a test agent (such as the binding domain) may be determined by any suitable method. Suitable methods include assaying for the ability of membrane-bound CD86 (e.g. on Raji cells) to bind and signal through CTLA-4 expressed on the surface of T cells, when in the presence of a test agent or in the presence of a suitable control. An increased level of T cell IL-2 production or an increase in T cell proliferation in the presence of the test agent relative to the level of T cell IL-2 production and/or T cell proliferation in the presence of the control is indicative of reduced signalling through CTLA-4 and increased cell activation. A typical assay of this type is disclosed in Example 9 of US20080233122.

### Binding Domains for Other T Cell Targets

The multispecific (e.g. bispecific) polypeptides of the invention also comprise a binding domain specific for a T cell target other than GITR and CTLA-4 (see above).

### (a) OX40-Binding Domains

In one embodiment, the multispecific (e.g. bispecific) polypeptide further comprises a binding domain specific for OX40.

Exemplary VH and VL regions of OX40-binding domains are disclosed in WO 2016/185016, the disclosures of which are incorporated by reference.

### (b) CD40-Binding Domains

In one embodiment, the multispecific (e.g. bispecific) polypeptide further comprises a binding domain specific for CD40.

Exemplary VH and VL regions of CD40-binding domains are shown in WO 2015/091853 and WO 2013/034904, the disclosures of which are incorporated herein by reference.

Embodiments of the Multispecific (e.g. Bispecific) Polypeptides of the Invention

In an embodiment of the first aspect of the invention, the bispecific polypeptide has binding domains which are specific for GITR and CTLA-4, for example B1 is specific for GITR and B2 is specific for CTLA-4.

These binding domains are as defined above.

### The Bispecific Polypeptide of the Embodiment Part B1—Binding Domain Specific for GITR

The binding domain specific for GITR is as defined above.

### The Bispecific Polypeptide of the Embodiment Part B2—Binding Domain Specific for CTLA-4

The binding domain specific for CTLA-4 is as defined above.

### The Bispecific Polypeptide of the Embodiment

The bispecific polypeptide of the invention is capable of specifically binding to both human GITR and human CTLA-4. By “capable of specifically binding to both GITR and CTLA-4”, it is meant that the anti-CTLA-4 part specifically binds to CTLA-4 and the anti-GITR part specifically binds to GITR, in accordance with the definitions provided for each part above. The bispecific polypeptide may comprise any GITR binding domain as described herein linked to any CTLA-4 binding domain as described herein. Preferably the binding characteristics of the different parts for their respective targets are unchanged or substantially unchanged when they are present as part of a bispecific antibody of the invention, when compared to said characteristics for the individual parts when present as separate entities.

Typically, this means that the bispecific molecule will have a Kd for CTLA-4 which is preferably substantially the same as the Kd value for CTLA-4 of the CTLA-4 binding domain when present alone. Alternatively, if the bispecific molecule has a Kd for CTLA-4 which is increased relative to the Kd for CTLA-4 of the CTLA-4 binding domain when present alone, then the increase is by no more than 10-fold, preferably no more than 9-fold, 8-fold, 7-fold, 6-fold, 5-fold, 4-fold, 3-fold or 2-fold. In addition, the bispecific molecule will independently have a Kd for GITR which is preferably substantially the same as the Kd value for GITR of the GITR binding domain when present alone. Alternatively, if the bispecific molecule has a Kd for GITR which is increased relative to the Kd for GITR of the anti-GITR antibody when present alone, then the increase is by no more than 10-fold, preferably no more than 9-fold, 8-fold, 7-fold, 6-fold, 5-fold, 4-fold, 3-fold or 2-fold. Preferred Kd values for the individual binding domains are as described above.

It will be appreciated that any of the fold changes in CTLA-4 binding may be independently combined with any of the recited fold changes in GITR binding to describe the binding characteristics of a given bispecific molecule.

The binding characteristics for GITR or CTLA-4 of any bispecific polypeptide of the invention may be assessed by any suitable assay. In particular, the assays set out above for each separate part may also be applied to a bispecific antibody or a combined assay to assess simultaneous binding to both targets may be used. Suitable assays for assessing the binding characteristics of bispecific polypeptides of the invention are also set out in the Examples, and are known in the art.

The bispecific polypeptide of the embodiment is capable of modulating the activity of cells of the immune system to a greater extent than an individual agonist of GITR or CTLA-4 alone, or than a combination of such individual agonists. In particular, administration of the bispecific polypeptide produces a higher level of T cell activity, in particular effector T cell activity, for example CD4+ effector T cell activity. The increase in effector T cell activity is also more localised than that which results from administration of an individual GITR or CTLA-4 agonist alone (or a combination thereof), because the bispecific polypeptide exerts the greatest effect only in a microenvironment in which CTLA-4 and GITR are both highly expressed. Tumours are such a microenvironment. GITR is expressed in elevated levels on CD8 T cells and may thus activate them in particular. CD8 T cells are one of the main effector component of an effective tumour response.

The increase in effector T cell activity may result directly from stimulation of the effector T cells via activation of the GITR pathway or via blockade of the CTLA-4 inhibition pathway, or may result indirectly from depletion or down-regulation of Tregs, thereby reducing their immunosuppressive effect. Depletion/down-regulation of Tregs may be mediated by ADCP or ADCC mechanisms. Overall, the result will be a very powerful, localised immune activation for the immediate generation of tumouricidal activity.

The cell surface expression pattern of CTLA-4 and GITR is partly overlapping, thus, the bispecific antibodies of the invention may bind to both targets both in cis and in trans. This may result in stimulation through GITR and CTLA-4 in an FcγR-cross-linking independent manner, either by increasing the level of receptor clustering in cis on the same cell, or by creating an artificial immunological synapse between two cells in trans, which in turn may lead to enhanced receptor clustering and increased signalling in both cells. Overall, the result will be a very powerful, tumour directed immune activation for the generation of tumouricidal activity.

Measurement of the effect of a bispecific polypeptide of the invention on cells of the immune system may be achieved with any suitable assay. For example, increased activity of effector T cells may be measured by assays as described above in respect of individual components B1 and B2 of the bispecific polypeptide, and include measurement of proliferation or IFNγ or IL-2 production by CD4+ and/or CD8+ T cells in the presence of the bispecific polypeptide relative to a control. An increase of proliferation or IFNγ or IL-2 production relative to control is indicative of increased cell activation. A typical assay of this type is disclosed in Example 9 of US20080233122. Assays for cell proliferation and/or IFNγ or IL-2 production are well known and are also exemplified in the Examples. When assessed in the same assay, the bispecific molecule will typically induce an increase in the activity of an effector T cell which is at least 1.5-fold higher or at least 2-fold higher, more preferably 3-fold higher, most preferably 5-fold higher than the increase in activity of an effector T cell induced by a combination of monospecific agents binding to the same targets.

The bispecific molecule potently activates the immune system when in a microenvironment in which both GITR and CTLA-4 are highly expressed. Typically, the bispecific molecule will increase the activity of a CD4+ or CD8+ effector cell, or may decrease the activity of a Treg cell. In either case, the net effect of the antibody will be an increase in the activity of effector T cells. When assessed in the same assay, the bispecific molecule will typically induce an increase in the activity of an effector T cell which is at least 1.5-fold higher or at least 1.7-fold higher, more preferably 4.5-fold higher, most preferably 7-fold higher than the increase in activity of an effector T cell induced by a combination of monospecific agents binding to the same targets.

Methods for determining a change in the activity of effector T cells are well known and are as described earlier. Assays for cell proliferation and/or IFNγ or IL-2 production are well known and are exemplified in the Examples.

For example, the polypeptide may be capable of specifically binding to both CTLA-4 and GITR, and B1 may be an antibody, or antigen binding fragment thereof, specific for GITR; and B2 may be a polypeptide binding domain specific for CTLA-4, which comprises or consists of:
i) the amino acid sequence of SEQ ID NO: 3; or
ii) an amino acid sequence in which at least one amino acid is changed when compared to the amino acid sequence of SEQ ID NO: 3 provided that said binding domain binds to human CTLA-4 with higher affinity than wild-type human CD86.

The CTLA-4 specifically bound by the polypeptide may be primate or murine, preferably human, CTLA-4, and/or the GITR specifically bound by the polypeptide may be primate, preferably human, GITR.

Part B1 of the polypeptide of the invention is an antibody, or antigen-binding fragment thereof, which typically comprises at least one heavy chain (H) and/or at least one light chain (L). Part B2 of the polypeptide of the invention may be attached to any part of B1, but may typically be attached to said at least one heavy chain (H) or at least one light chain (L), preferably at either the N or the C terminus. Part B2 of the polypeptide of the invention may be so attached either directly or indirectly via any suitable linking molecule (a linker).

Part B1 preferably comprises at least one heavy chain (H) and at least one light chain (L) and part B2 is preferably attached to the N or the C terminus of either said heavy chain (H) or said light chain (L). An exemplary antibody of B1 consists of two identical heavy chains (H) and two identical light chains (L). Such an antibody is typically arranged as two arms, each of which has one H and one L joined as a heterodimer, and the two arms are joined by disulfide bonds between the H chains. Thus, the antibody is effectively a homodimer formed of two H-L heterodimers. Part B2 of the polypeptide of the invention may be attached to both H chains or both L chains of such an antibody, or to just one H chain, or just one L chain.

The polypeptide of the invention may therefore alternatively be described as an anti-GITR antibody, or an antigen binding fragment thereof, to which is attached at least one polypeptide binding domain specific for CTLA-4, which comprises or consists of the monomeric soluble extracellular domain of human wild-type CD86 or a variant thereof. The binding domains of B1 and B2 may be the only binding domains in the polypeptide of the invention.

The polypeptide of the invention may comprise a polypeptide arranged according to any one of the following formulae, written in the direction N-C:

L-(X)n-B2; (A)

B2-(X)n-L; (B)

B2-(X)n-H; (C)

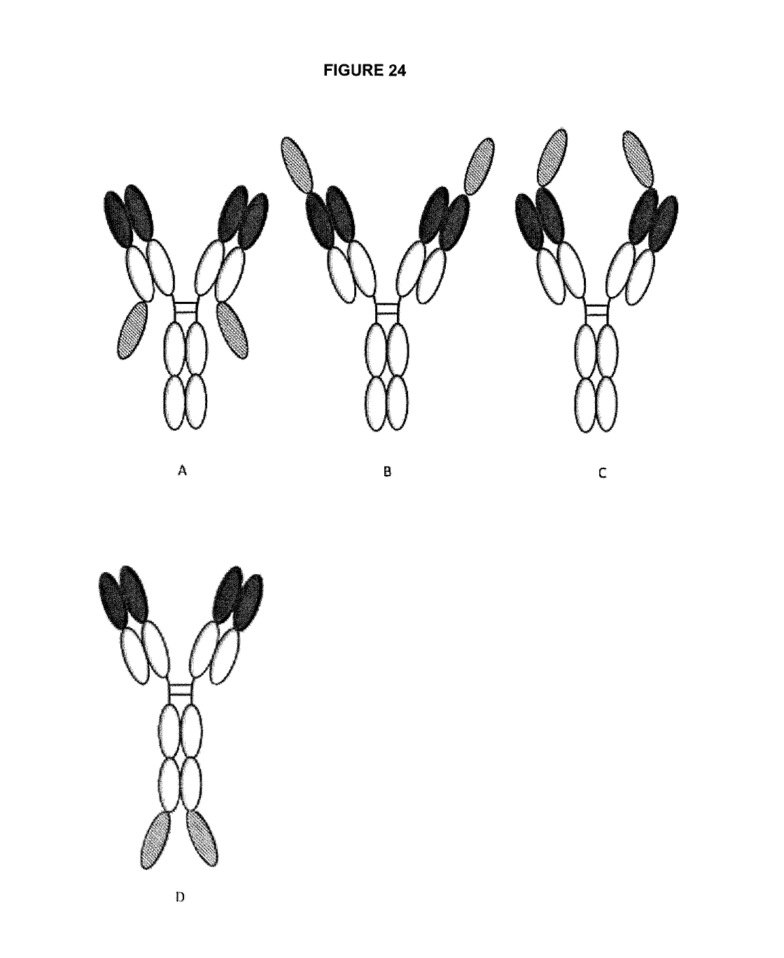

H-(X)n-B2; (D)

wherein H is the heavy chain of an antibody (i.e. of B1), L is the light chain of an antibody (i.e. of B1), X is a linker and n is 0 or 1. Where the linker (X) is a peptide, it typically has the amino acid sequence SGGGGSGGGGS (SEQ ID NO: 47), SGGGGSGGGGSAP (SEQ ID NO: 48), NFSQP (SEQ ID NO: 49), KRTVA (SEQ ID NO: 50), GGGGSGGGGSGGGGS (SEQ ID NO: 51) or (SG)m, where m=1 to 7. Schematic representations of formulae (A) to (D) are shown in FIG. 24.

The present invention also provides a polypeptide which consists of a polypeptide arranged according to any of formulae (A) to (D). Said polypeptide may be provided as a monomer or may be present as a component of a multimeric protein, such as an antibody. Said polypeptide may be isolated. Examples of amino acid sequences of such polypeptides are shown in Table C. Exemplary nucleic acid sequences encoding each amino acid sequence are also shown. Exemplary amino acid and nucleotide sequences are recited in SEQ ID NOs 68-75.

Part B2 may be attached to any part of a polypeptide of the invention, or to a linker, by any suitable means. For example, the various parts of the polypeptide may be joined by chemical conjugation, such as with a peptide bond. Thus, the polypeptide of the invention may comprise or consist of a fusion protein comprising B1 (or a component part thereof) and B2, optionally joined by a peptide linker. In such a fusion protein, the GITR-binding domain or domains of B1 and the CTLA-4-binding domain or domains of B2 may be the only binding domains.

Other methods for conjugating molecules to polypeptides are known in the art. For example, carbodiimide conjugation (see Bauminger & Wilchek, 1980, Methods Enzymol. 70:151-159; the disclosures of which are incorporated herein by reference) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety. As a further example, conjugation may be achieved by sodium periodate oxidation followed by reductive alkylation of appropriate reactants, or by glutaraldehyde cross-linking. However, it is recognised that, regardless of which method is selected, a determination should preferably be made that parts B1 and B2 retain or substantially retain their target binding properties when present as parts of the polypeptide of the invention.

The same techniques may be used to link the polypeptide of the invention (directly or indirectly) to another molecule. The other molecule may be a therapeutic agent or a detectable label. Suitable therapeutic agents include a cytotoxic moiety or a drug.

A polypeptide of the invention may be provided in isolated or substantially isolated form. By substantially isolated, it is meant that there may be substantial, but not total, isolation of the polypeptide from any surrounding medium. The polypeptides may be mixed with carriers or diluents which will not interfere with their intended use and still be regarded as substantially isolated.

Exemplary polypeptides of the invention may comprise or consist of any one of the amino acid sequences shown in Table C.

Representative polynucleotides which encode examples of a heavy chain or light chain amino acid sequence of an antibody may comprise or consist of any one of the nucleotide sequences set out in Table C as SEQ ID NOs 53, 55, 57, 59, 60, 62, 64, or 66. Representative polynucleotides which encode the polypeptides shown in Table C may comprise or consist of the corresponding nucleotide sequences which are also shown in Table C (intron sequences are shown in lower case) (For example, SEQ ID NOs 68, 70, 72, and 74). Representative polynucleotides which encode examples of part B2 may comprise or consist of any one of SEQ ID NOS: 25 to 43 as shown in Table B.

### Further Aspects of the Invention

A second aspect of the invention comprises a multispecific (e.g. bispecific) polypeptide according to the first aspect of the invention for use in a method for treating or preventing a disease or condition in an individual, as described above.

A third aspect of the invention is a method of treating or preventing a disease or condition in an individual, the method comprising administering to an individual a multispecific (e.g. bispecific) polypeptide according to the first or second aspects of the invention, as described above.

One embodiment of the invention is a multispecific (e.g. bispecific) polypeptide according to the second aspect of the invention or a method according to third aspect of the invention wherein the disease or condition is cancer and optionally wherein the individual is human.

In a further embodiment, the method comprises administering the multispecific (e.g. bispecific) antibody systemically or locally, such as at the site of a tumour or into a tumour draining lymph node, as described above.

The cancer may be prostate cancer, breast cancer, colorectal cancer, pancreatic cancer, ovarian cancer, lung cancer, cervical cancer, rhabdomyosarcoma, neuroblastoma, multiple myeloma, leukemia, acute lymphoblastic leukemia, melanoma, bladder cancer, gastric cancer, head and neck cancer, liver cancer, skin cancer, lymphoma or glioblastoma.

A fourth aspect of the invention is a polynucleotide encoding at least one polypeptide chain of a multispecific (e.g. bispecific) polypeptide according to the first or second aspects of the invention, as described above.

A fifth aspect of the invention is a composition comprising a multispecific (e.g. bispecific) polypeptide according to the first or second aspects of the invention and at least one pharmaceutically acceptable diluent or carrier.

In one embodiment of the invention a polypeptide according to either the first or second aspect of the embodiment is conjugated to an additional therapeutic moiety.

It will also be appreciated by persons skilled in the art that the pharmaceutical compositions of the invention may be administered alone or in combination with other therapeutic agents used in the treatment of cancers, such as antimetabolites, alkylating agents, anthracyclines and other cytotoxic antibiotics, vinca alkyloids, etoposide, platinum compounds, taxanes, topoisomerase I inhibitors, antiproliferative immunosuppressants, corticosteroids, sex hormones and hormone antagonists, and other immunotherapeutic antibodies (such as trastuzumab).

The combination therapies of the invention may additionally comprise a further immunotherapeutic agent, effective in the treatment of cancer, which specifically binds to an immune checkpoint molecule other than GITR and/or CTLA-4. It will be appreciated that the therapeutic benefit of the further immunotherapeutic agent may be mediated by attenuating the function of an inhibitory immune checkpoint molecule and/or by activating the function of a stimulatory immune checkpoint molecule.

In another embodiment, the additional therapeutic moiety is an immunotherapeutic agent selected from the groups consisting of:
(a) an immunotherapeutic agent that binds PD-1;
(b) an immunotherapeutic agent that binds OX40; and
(c) an immunotherapeutic agent that binds CD137.

Thus, the further immunotherapeutic agent may be a PD1 inhibitor, such as an anti-PD1 antibody, or antigen-binding fragment thereof capable of inhibiting PD1 function (for example, Nivolumab, Pembrolizumab, Lambrolizumab, Pidilzumab and AMP-224). Alternatively, the PD1 inhibitor may comprise or consist of an anti-PD-L1 antibody, or antigen-binding fragment thereof capable of inhibiting PD1 function (for example, MEDI-4736 and MPDL3280A).

A sixth aspect of the invention is an antibody specific for GITR which is as defined earlier.

Preferred, non-limiting examples which embody certain aspects of the invention will now be described, with reference to the following figures:

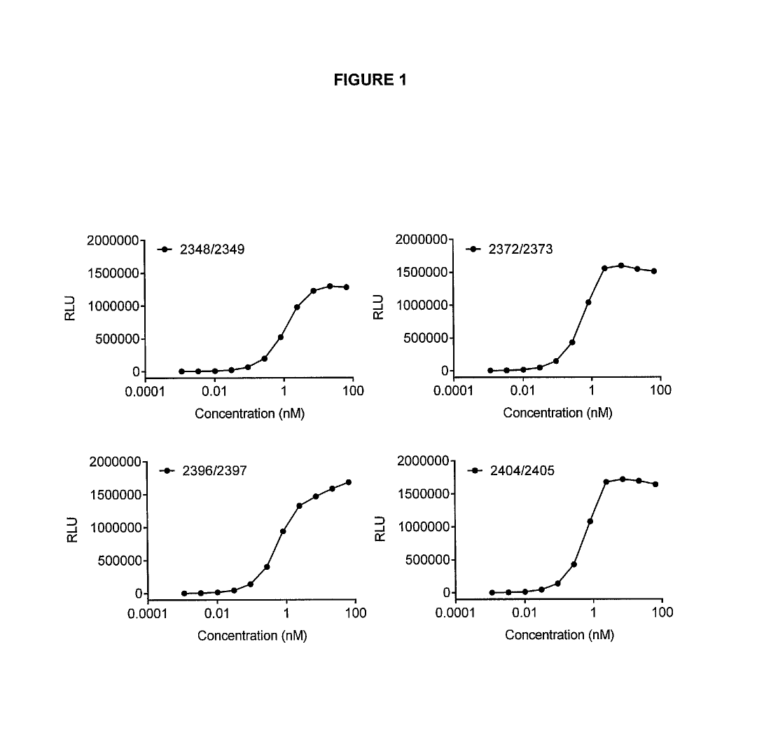

FIG. 1 shows dual antigen binding by a range of different bispecific antibodies. Human GITR was coated in ELISA plates, and the bispecific antibodies added at different concentrations. Biotinylated CTLA-4 was added as secondary antigen and Streptavidin-HRP used as a detection reagent.

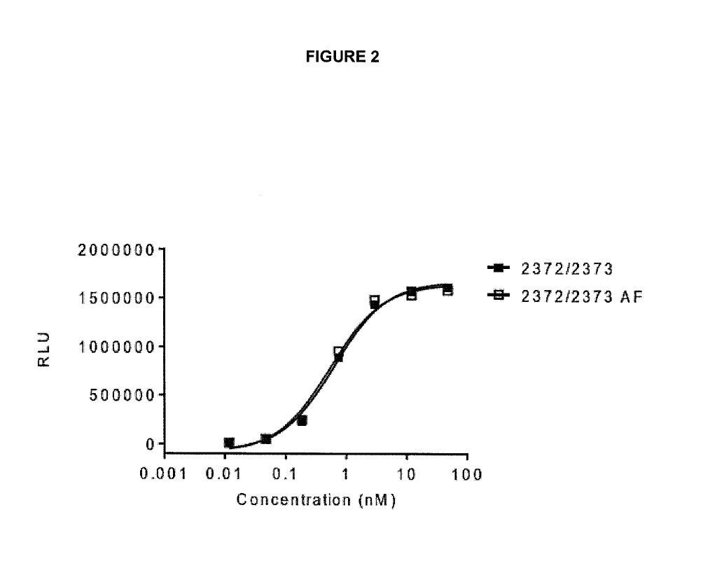

FIG. 2 shows dual antigen binding by GITR/CTLA-4 bispecific antibody 2372/2373 in wildtype and afucosylated format. Human GITR was coated in ELISA plates, and the antibodies added at different concentrations. Biotinylated CTLA-4 was added as secondary antigen and Streptavidin-HRP used as a detection reagent.

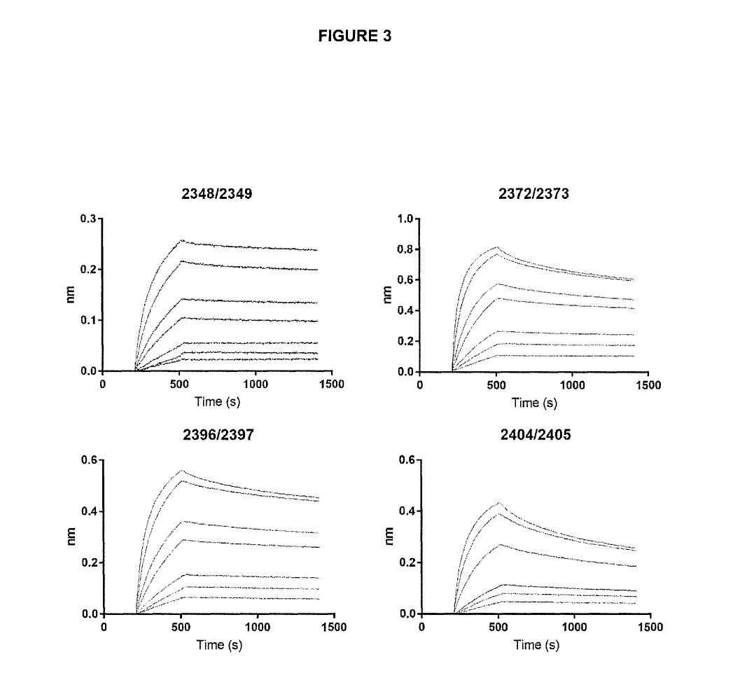

FIG. 3 shows kinetic profiles of bispecific antibodies interacting with human GITR. The bispecific antibodies were assayed (300 sec association and 900 sec dissociation) against GITR immobilized on sensor tip surfaces at concentrations ranging from 1.25 to 80 nM.

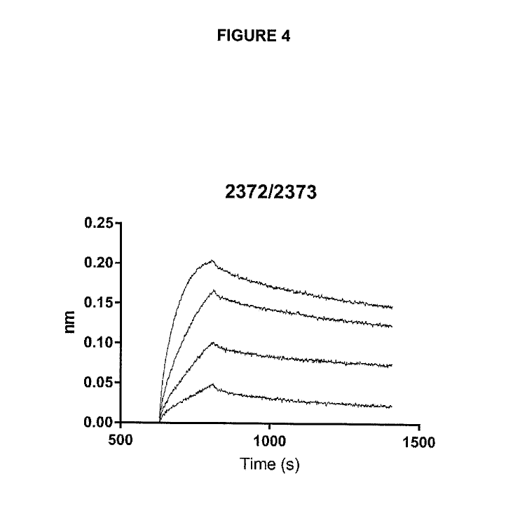

FIG. 4 shows the kinetic profile of bispecific antibody 2372/2373 interacting with human CTLA-4. The bispecific antibody was immobilized on sensor tips and assayed (180 sec association and 600 sec dissociation) against hCTLA-4 at concentrations ranging from 10 to 80 nM.

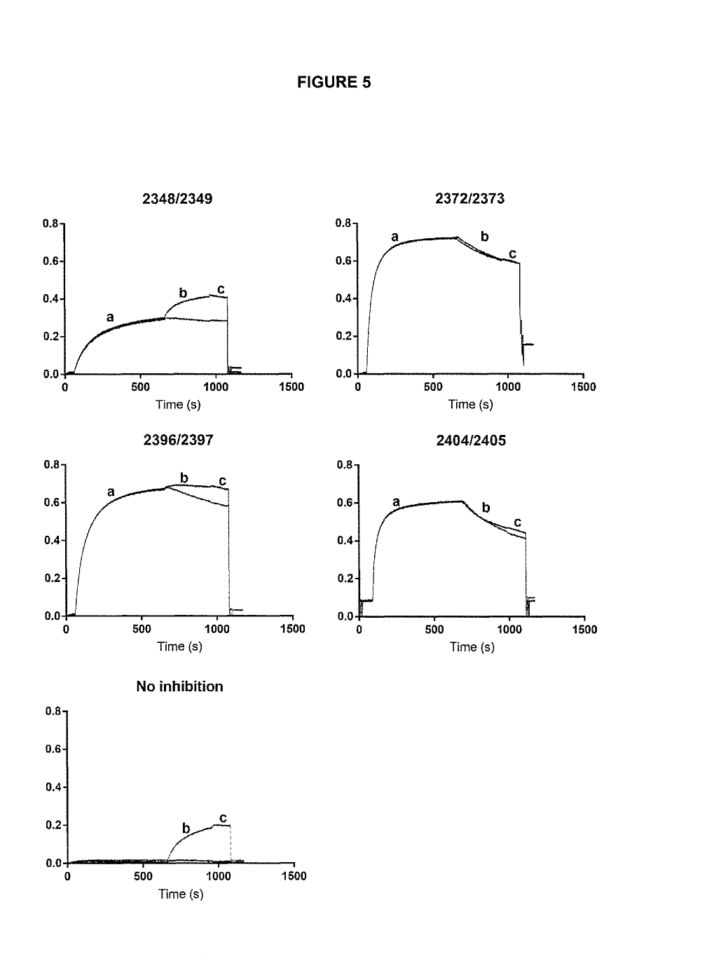

FIG. 5 shows the ability of bispecific antibodies to block GITR-GITR Ligand interactions. The top four subfigures show sensograms from the two sensor tips used for each bispecific antibody (assay sensor and reference sensor) and the bottom subfigure shows binding of GITR Ligand to GITR without the presence of any bispecific antibody. The different steps included in the figure are a) binding of bispecific antibody to immobilized GITR, b) either binding of GITR Ligand to immobilized GITR (assay sensor) or dissociation of bound bispecific antibodies in kinetics buffer (reference sensor) and c) dissociation of formed GITR-GITR Ligand complexes.

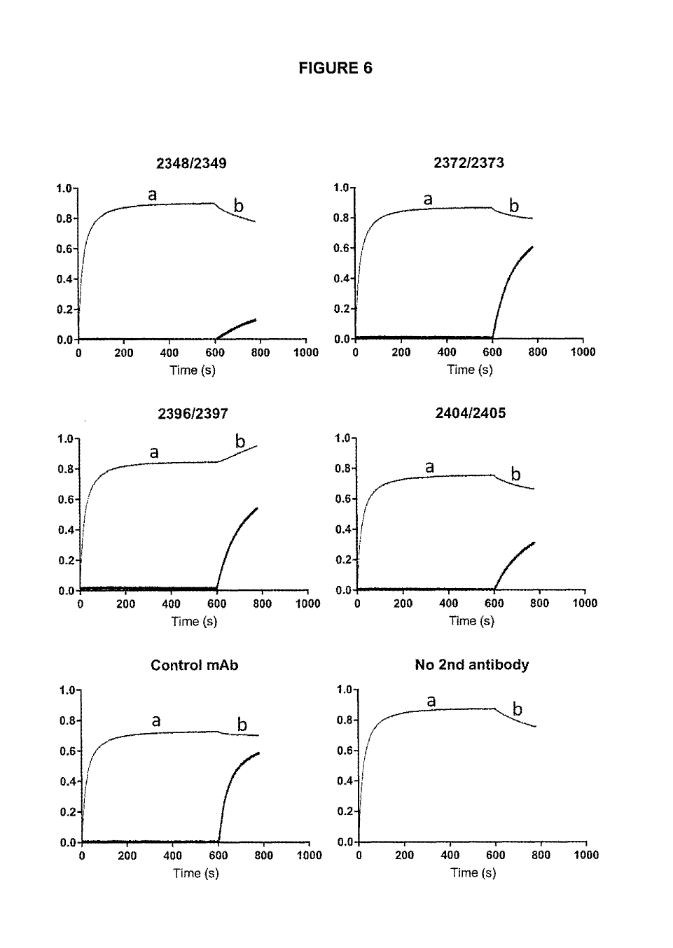

FIG. 6 shows the ability of bispecific antibody 2372/2373 to block interaction of secondary antibodies (bispecific or monospecific) with GITR. The top four subfigures show sensograms from the two sensor tips used for each secondary bispecific antibody (assay sensor and reference sensor), the bottom left subfigure shows sensor tips used for the control mAb and the bottom right subfigure shows the association and dissociation profile of 2372/2373 without any secondary antibody. The different steps included in the figure are a) binding of bispecific antibody 2372/2373 to immobilized GITR, b) binding of secondary antibody to immobilized GITR with (assay sensor, top sensogram) or without (reference sensor, bottom sensogram) prior blocking with 2372/2373.

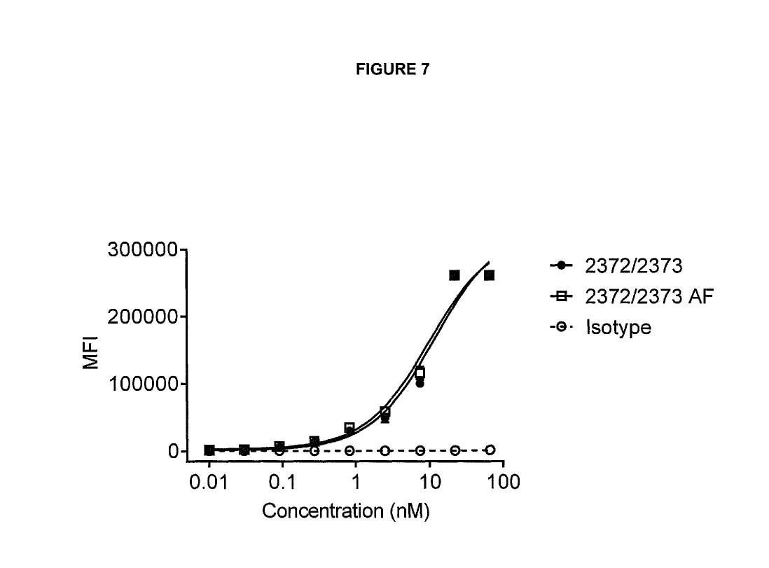

FIG. 7 shows the binding of GITR/CTLA-4 bispecific antibody 2372/2373 in wildtype and afucosylated format to target-expressing cells, as determined by flow cytometry. CHO-GITR^{hi}-CTLA-4^{hi} cells were stained with serially diluted antibody followed by a secondary PE-conjugated anti-h Fc antibody.

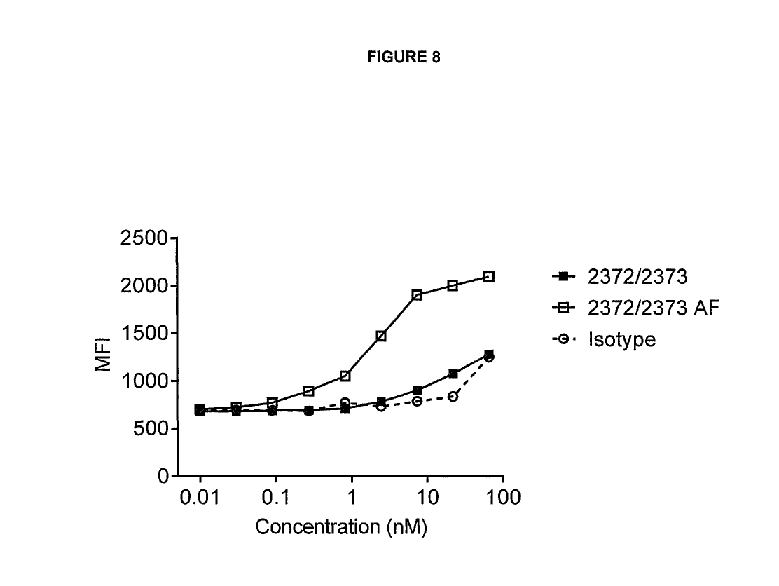

FIG. 8 shows the binding of GITR/CTLA-4 bispecific antibody 2372/2373 in wildtype and afucosylated format to FcγRIIIa-expressing cells was determined by flow cytometry. CHO-FcγRIIIa cells were stained with serially diluted antibodies followed by a secondary PE-conjugated anti-hFc antibody.

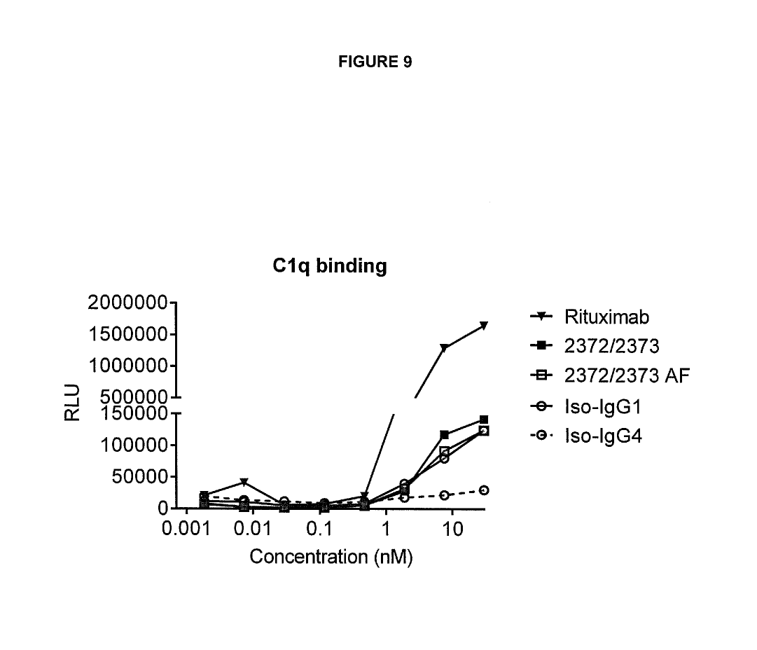

FIG. 9 shows binding to C1q of wildtype and afucosylated 2372/2373 GITR/CTLA-4 bispecific antibodies, assessed using ELISA. Human C1q was coated onto the plate, and the antibodies were added at different concentrations. A sheep anti-human C1q-HRP was used as detection antibody, followed by peroxidase substrate. Rituximab was included as a positive control, and IgG1 and IgG4 isotype controls as negative controls.

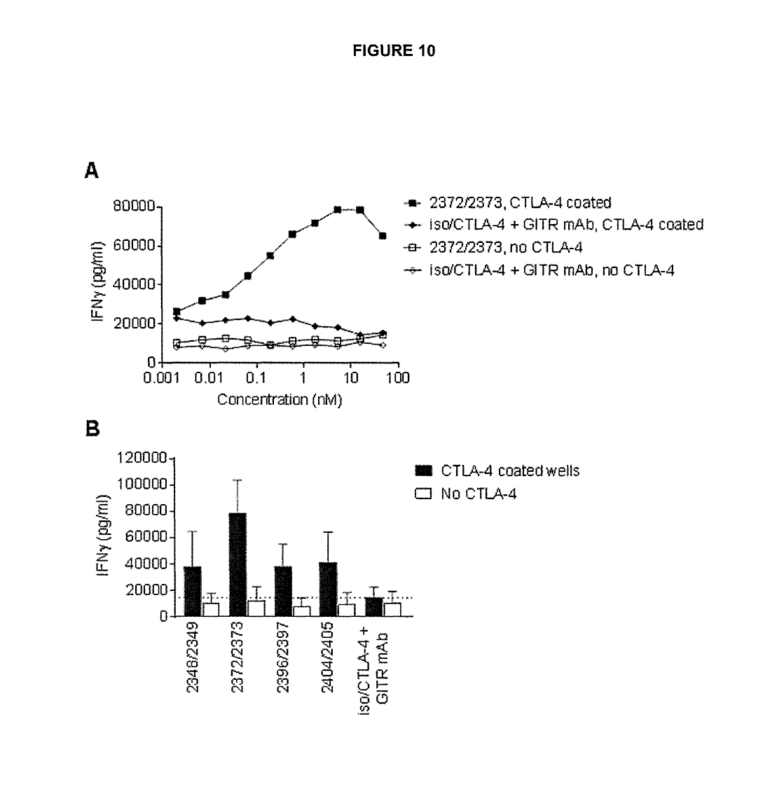

FIG. 10 shows IFNγ production following stimulation in vitro of human CD3 positive T cells stimulated with either soluble GITR/CTLA-4 bispecific antibodies or the combination of soluble monospecific controls (a GITR mAb from Miltenyi and an isotype control with the CTLA-4 binding part, iso/CTLA-4). The experiment was performed in plates coated with CD3 with or without CTLA-4. A) A full dose-response curve of the GITR/CTLA-4 bispecific antibody: 2372/2373. B) A single antibody concentration (16 nM) of the bispecific antibodies: 2348/2349, 2372/2373, 2396/2397 and 2404/2405 or monospecific controls. The assay was performed twice in a total of 4 donors. One representative experiment (mean of 2 donors) is shown.

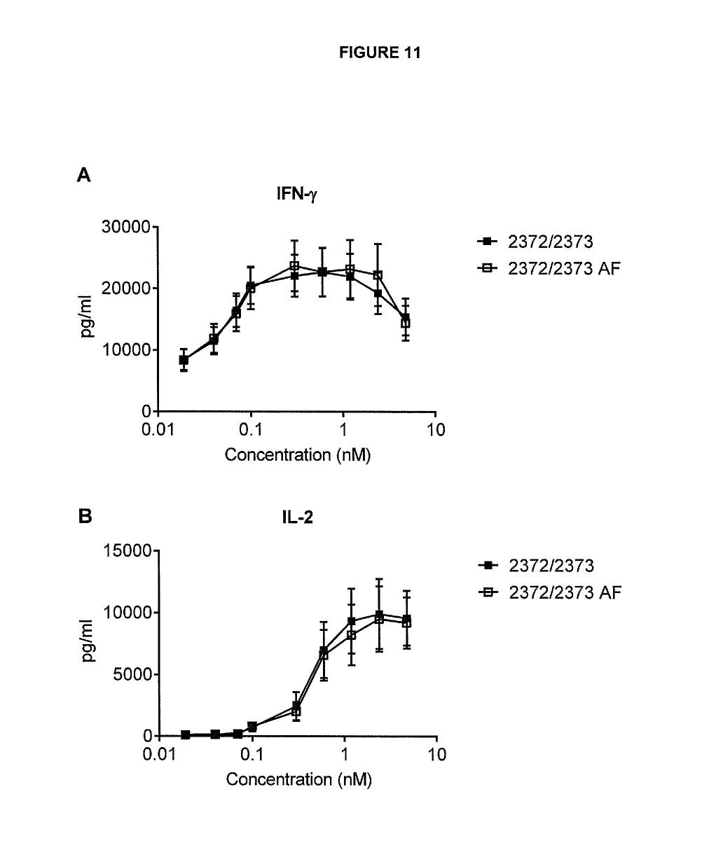

FIG. 11 shows the agonistic effect of the wildtype and the afucosylated 2372/2373 variant. CD3⁺ T cells were stimulated with wildtype and afucosylated GITR/CTLA-4 bispecific antibodies for 72 h in plates coated with αCD3 and CTLA-4. Secretion of (A) IFN-γ, and (B) IL-2 were measured in the supernatants by ELISA. One representative experiment (mean of 4 donors) is shown.

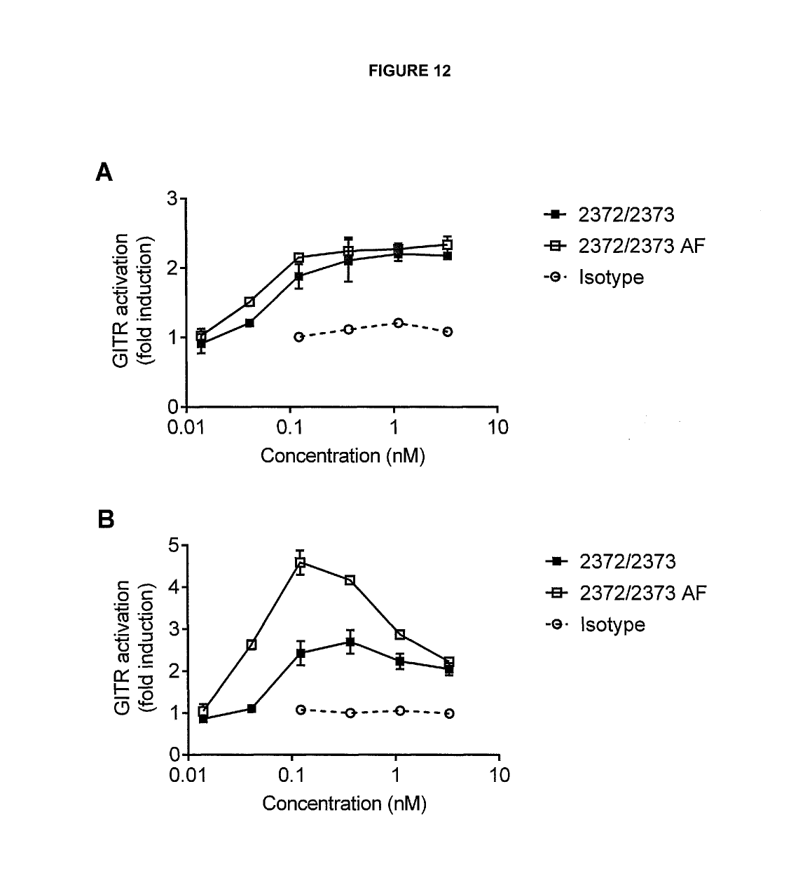

FIG. 12 shows GITR activation in response to wildtype and afucosylated GITR/CTLA-4 bispecific antibody 2372/2373 and isotype control A) in the absence of FcγRIIIa expressing cells, and B) in the presence of FcγRIIIa expressing CHO cells (100,000 cells/well). GITR expressing Jurkat cells were used as reporter cells. Data is presented as fold induction over medium control.

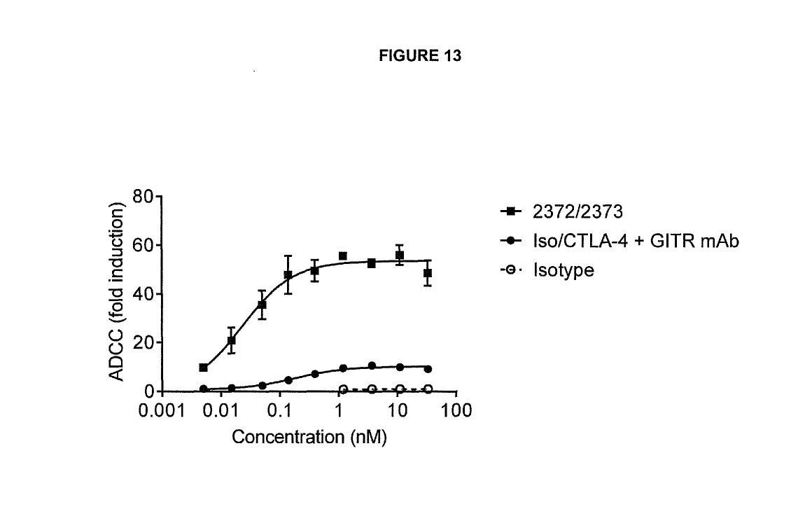

FIG. 13 shows activation of FcγRIIIa (V158) effector cells in response to the GITR/CTLA-4 bispecific antibody 2372/2373, the combination of monospecific counterparts (iso/CTLA-4⁺αGITR mAb) and isotype control. GITR^{hi}-CTLA4^{lo} CHO cells were used as target cells. Data is presented as fold induction over medium control. One out of two experiments is shown.

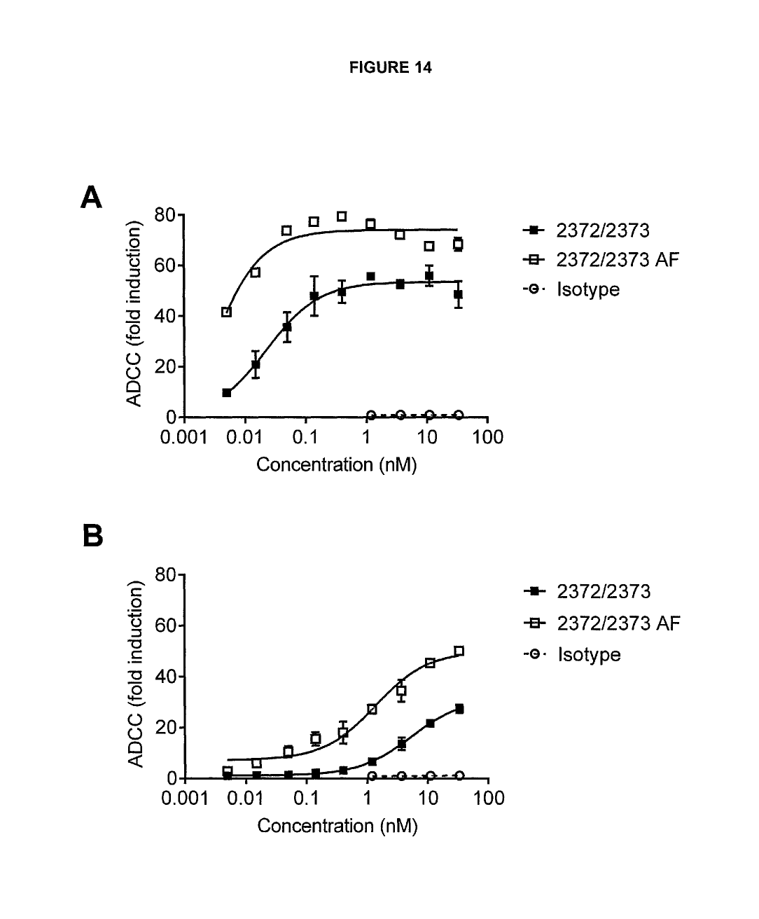

FIG. 14 shows activation of FcγRIIIa (V158) effector cells in response to wildtype and afucosylated 2372/2373 GITR/CTLA-4 bispecific antibody and isotype control. As target cells, A) CHO-GITR^{hi}-CTLA4^{lo} cells, and B) CHO-GITR^{hi}-CTLA4^{hi} cells were used. Data is presented as fold induction over medium control. One out of two experiments is shown.

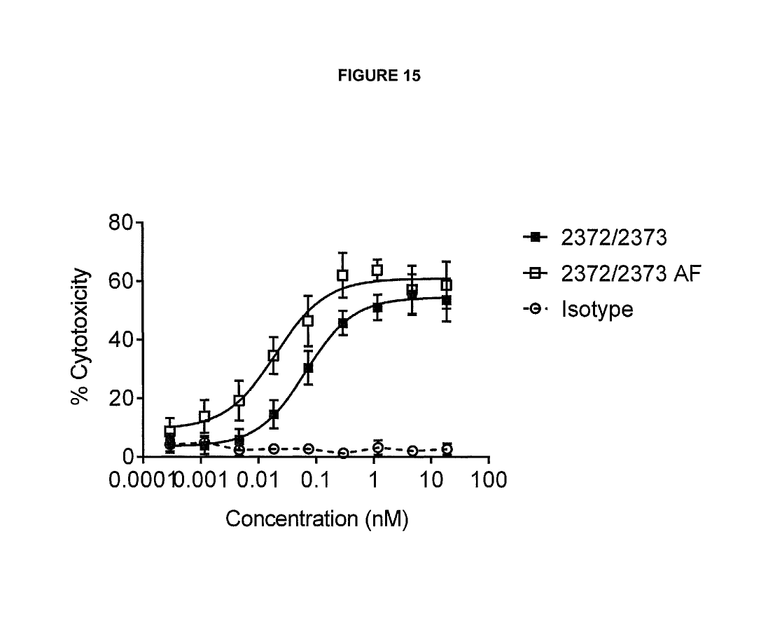

FIG. 15 shows ADCC in response to wildtype and afucosylated GITR/CTLA-4 bispecific antibodies 2372/2373 and isotype control. PBMC effector cells and CHO-GITR^{hi}-CTLA4^{hi} cells as target cells were co-cultured at a 50:1 ratio with test compounds for 4 h before measurements of LDH in the supernatants. The mean of 4 donors is shown.

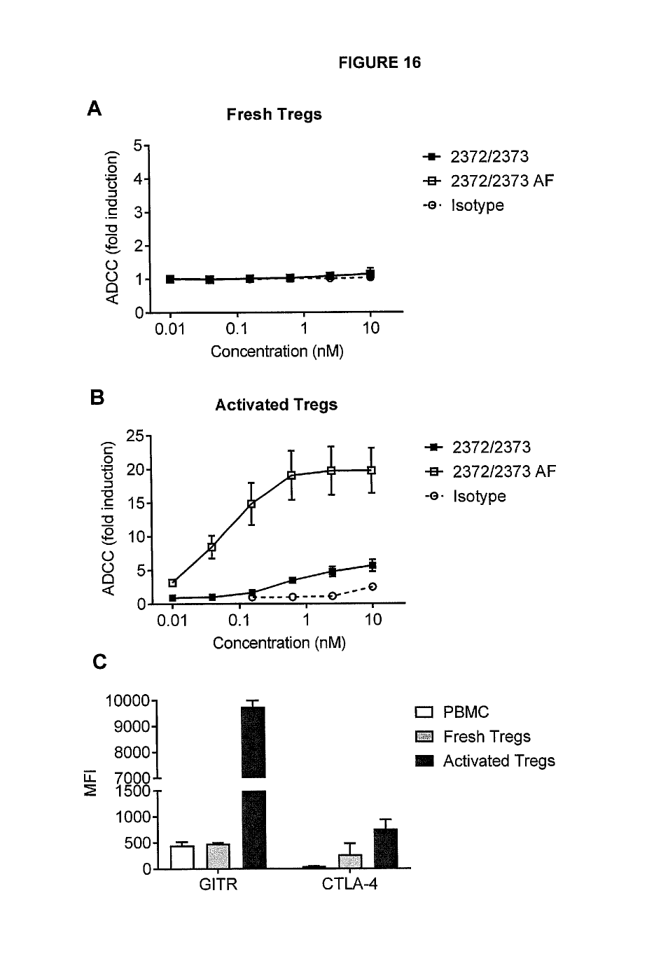

FIG. 16 shows activation of FcγRIIIa (V158) effector cells in response to wildtype and afucosylated 2372/2373 GITR/CTLA-4 bispecific antibodies. As target cells, (A) freshly isolated Tregs (CD4⁺CD25⁺CD127^{lo}), and (B) Tregs activated for 48 h with αCD3/αCD28 beads were used. Data is presented as fold induction over medium control. (C) Expression of GITR and CTLA-4 was determined by flow cytometry on PBMC and Tregs before and after activation. The mean of two donors is shown.

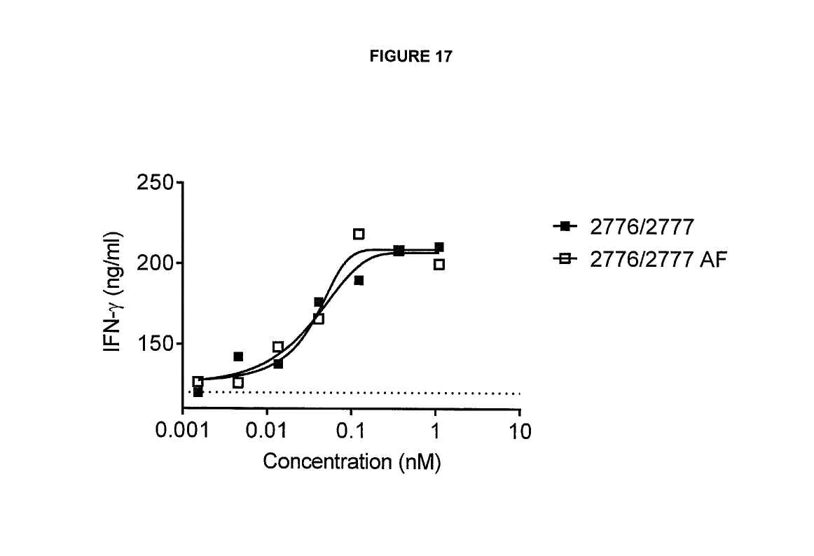

FIG. 17 shows agonistic effects of the surrogate bispecific antibodies in splenocyte assay. CD3⁺ T cells were stimulated with wildtype or afucosylated GITR/CTLA-4 bispecific antibodies for 48 h in plates coated with αCD3 and CTLA-4, and the activation of T-cells was measured in form of IFN-γ secretion by ELISA.

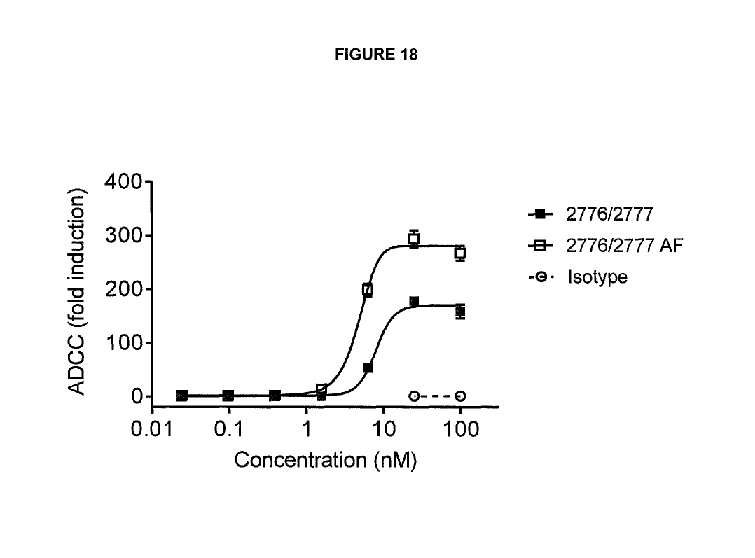

FIG. 18 shows activation of mFcγRIV reporter cells as an indicator for ADCC response by the surrogate wildtype or afucosylated GITR/CTLA-4 bispecific antibodies. Data is presented as fold induction over medium control.

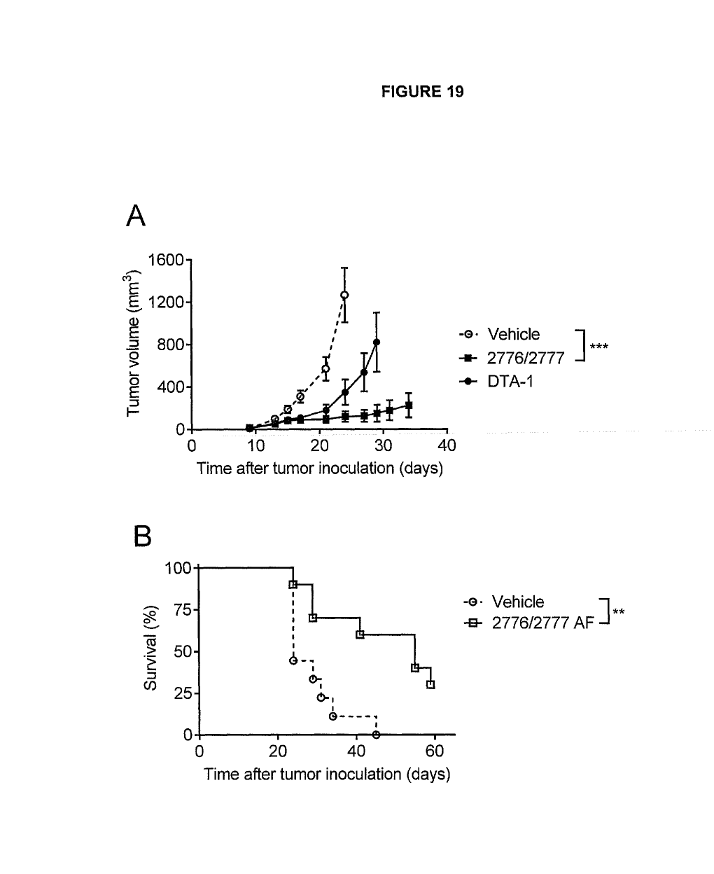

FIG. 19 shows anti-tumor effects of bispecific surrogate GITR/CTLA-4 antibodies in CT26 colon carcinoma model. Intraperitoneal treatments were done on days 7, 10 and 13. (A) Tumor volume inhibition by 2776/2777 compared to vehicle and DTA-1. (B) Increased survival of 2776/2777 AF compared to vehicle. The graphs shown exemplary graph, mean tumor volume +/−SEM or Kaplan-Meyer survival, n=10/experiment.

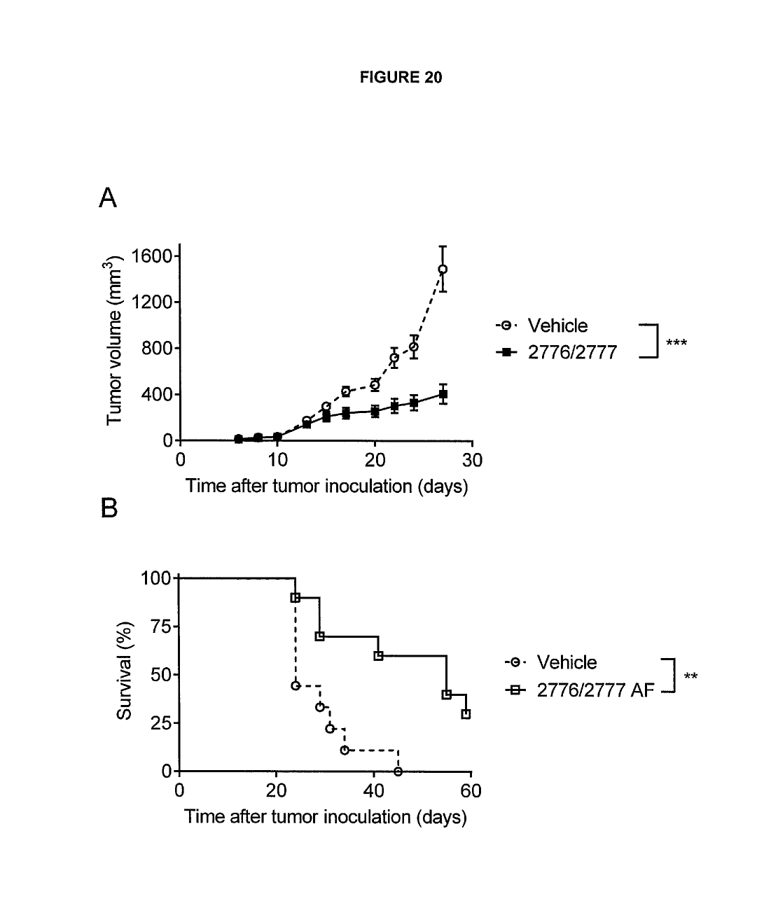

FIG. 20 shows anti-tumor effects of bispecific surrogate GITR/CTLA-4 antibodies in MC38 colon carcinoma model. Treatments were done intraperitoneally on days 7, 10 and 13 on mice bearing established subcutaneous tumors. (A) Tumor volume inhibition by 2776/2777, (B) Increased survival of 2776/2777 AF treated mice compared to vehicle. The graphs show exemplary graph, mean tumor volume +/−SEM, or Kaplan-Meyer survival, n=10/experiment.

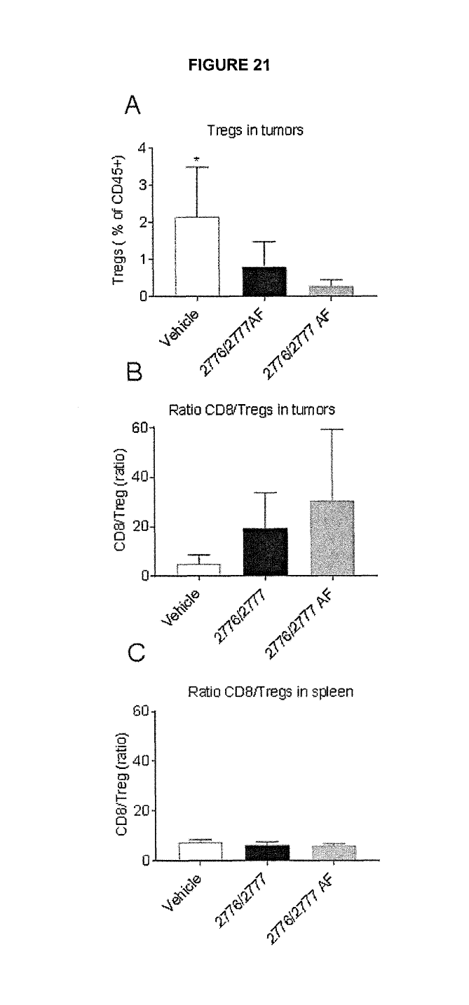

FIG. 21 shows anti-tumor effects of bispecific surrogate antibodies on Tregs. Mice bearing subcutaneous MC38 colon carcinoma were treated with intraperitoneal injections with 2776/2777 or 2776/2777 AF (200 μg) on days 10, 13 and, 16. Twenty-four hours after the last injection, the tumors and spleens were harvested, and stained for Tregs and effector cells. (A) Percent Tregs in tumors (B) Intratumoral CD8/Treg ratio, and (C) CD8/Treg ratio in spleens. The graphs show mean+SD.

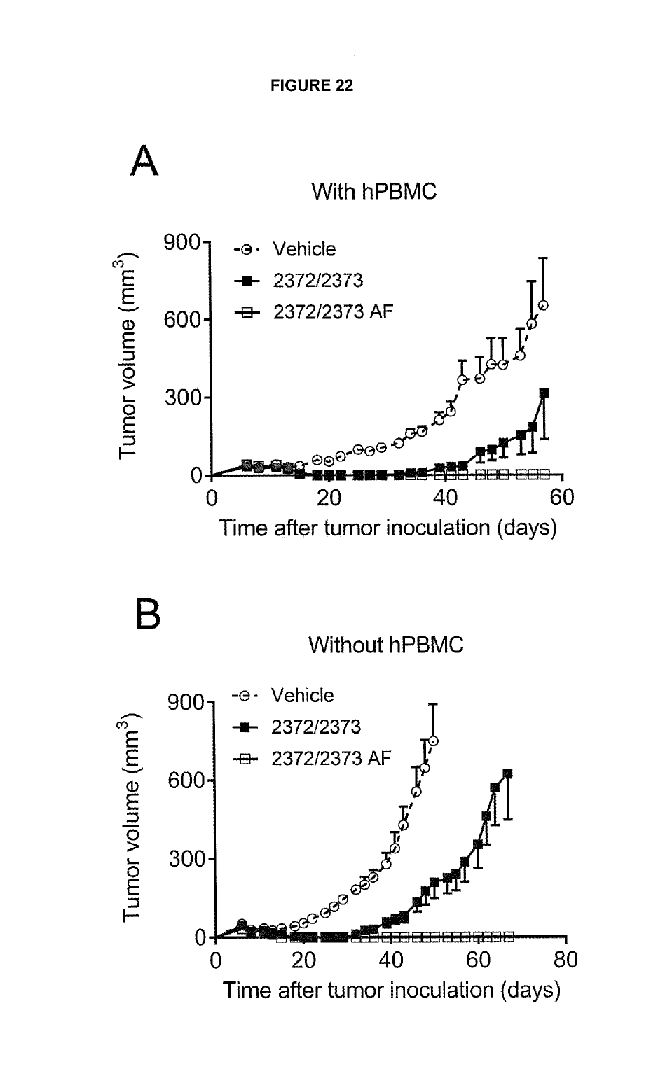

FIG. 22 shows anti-tumor efficacy of bispecific GITR/CTLA-4 bispecific antibodies. RPMI-8226 plasmacytoma (10×10⁶) was inoculated subcutaneously to the right hind flank/back at day 0. Human PBMC cells (5×10⁶) were administered intraperitoneally on day 5. The treatments were done by intraperitoneal injections (app 500 nmol/dose) on days 5, 11 and 18. (A) Tumor volume inhibition in the presence of hPBMC, n=5/donor, n(donor)=2 (B) Tumor volume inhibition without hPBMC, n=10/group. The graphs show the mean+/−SEM.

FIG. 23 provides a schematic representation of human wild-type CD86 amino acid sequences disclosed herein. (A) is the amino acid sequence of the monomeric soluble extracellular domain of human CD86 without N-terminal signal sequence (SEQ ID NO: 3); (B) is the amino acid sequence of the monomeric extracellular and transmembrane domains of human wildtype CD86, including N-terminal signal sequence (SEQ ID NO: 4); (C) is the full length amino acid sequence of human CD86 (Genbank ABK41931.1; SEQ ID NO: 44). The sequence in A may optionally lack Alanine and Proline at the N terminus, i.e. positions 24 and 25, shown in bold. Signal sequences in B and C are underlined. Numbering of amino acid positions is based on SEQ ID NOs: 4 and 44, starting from the N terminus.

FIG. 24 shows a schematic representation of the structure of exemplary arrangements for the bispecific polypeptides of the invention. Anti-GITR antibody variable domains are filled in black; constant domains in white. CTLA-A binding domains are shaded with diagonal lines.

### DESCRIPTION OF THE SEQUENCES

SEQ ID NO: 1 is the amino acid sequence of human CTLA-4 (corresponding to GenBank: AAD00698.1)

SEQ ID NO: 2 is the amino acid sequence of human CD28 (corresponding to GenBank: AAA51944.1)

SEQ ID NO: 3 is the amino acid sequence of the monomeric extracellular domain of human wildtype CD86, excluding a 23-amino acid signal sequence from the N terminus.

SEQ ID NO: 4 is the amino acid sequence of the monomeric extracellular and transmembrane domains of human wildtype CD86, including N-terminal signal sequence (see FIG. 23). All numbering of amino acid positions herein is based on the positions in SEQ ID NO: 4 starting from the N terminus. Thus, the Alanine at the N terminus of SEQ ID NO: 3 is numbered 24.

SEQ ID NO: 5 is the amino acid sequence of a mutant form of the extracellular domain of human CD86 disclosed in Peach et al (Journal of Biological Chemistry 1995, vol 270(36), 21181-21187). H at position 79 of the wild type sequence is substituted with A in the corresponding position for the sequence of SEQ ID NO: 5. This change is referred to herein as H79A. Equivalent nomenclature is used throughout for other amino acid substitutions referred to herein. Numbering of positions is based on SEQ ID NO: 4 as outlined above.

SEQ ID NOs: 6 to 24 are the amino acid sequences of specific proteins of the invention.

SEQ ID NOs: 25 to 43 are nucleotide sequences encoding the amino acid sequences of each of SEQ ID NOs 6 to 24, respectively

SEQ ID NO: 44 is the full length amino acid sequence of human CD86 (corresponding to GenBank: ABK41931.1)

SEQ ID NO: 45 is the amino acid sequence of murine CTLA-4 (corresponding to UniProtKB/Swiss-Prot: P09793.1).

SEQ ID NO: 46 is the amino acid sequence of murine CD28 (corresponding to GenBank: AAA37395.1).

SEQ ID NOs: 47 to 51 are various linkers which may be used in the bispecific polypeptides of the invention.

SEQ ID NOs: 52 to 75 are exemplary sequences of the invention.

SEQ ID NOs: 76 to 96 are exemplary CDR sequences of the invention.

SEQ ID NO: 97 is an exemplary heavy chain constant region amino acid sequence.

SEQ ID NO: 98 is an exemplary light chain constant region amino acid sequence.

SEQ ID NO: 99 is an exemplary modified human heavy chain IgG4 constant region sequence with a mutation from Ser to Pro in the hinge region (position 108) and from His to Arg in the CH3 region (position 315). Mutations result in reduced serum half-life and stabilization of the core hinge of IgG4 making the IgG4 more stable, preventing Fab arm exchange.

SEQ ID NO: 100 is an exemplary wild type human heavy chain IgG4 constant region sequence. That is a sequence lacking the mutations of SEQ ID NO: 99.

SEQ ID NO: 101 is an exemplary modified human heavy chain IgG4 constant region sequence with a single mutation from Ser to Pro in the hinge region (position 108). Mutation results in stabilization of the core hinge of IgG4 making the IgG4 more stable, preventing Fab arm exchange.

SEQ ID NO: 102 is an exemplary cDNA sequence (i.e. lacking introns) encoding the IgG4 constant region of SEQ ID NO: 99.

SEQ ID NO: 103 is an exemplary genomic DNA sequence (i.e. including introns) encoding the IgG4 constant region of SEQ ID NO: 99

SEQ ID NO: 104 is an exemplary cDNA sequence (i.e. lacking introns) encoding the IgG4 constant region of SEQ ID NO: 100.

SEQ ID NO: 105 is an exemplary genomic DNA sequence (i.e. including introns) encoding the IgG4 constant region of SEQ ID NO: 100.

SEQ ID NOs: 106 and 107 are exemplary cDNA and genomic DNA sequences, respectively, encoding the IgG1 constant region of SEQ ID NO: 97.

SEQ ID NOs: 108 is an exemplary DNA sequence encoding the light chain kappa region of SEQ ID NO: 98.

SEQ ID NO: 109 is an exemplary cDNA sequence (i.e. lacking introns) encoding the IgG4 region of SEQ ID NO: 101.

SEQ ID NO: 110 is an exemplary genomic DNA sequence (i.e. including introns) encoding the IgG4 region of SEQ ID NO: 101.

SEQ ID NO: 111 is the amino acid sequence of human GITR (corresponding to GenBank: AAD00698.1)

SEQ ID NOs: 112 to 143 are exemplary amino acid and nucleotide sequences of VL and VH regions of OX40-binding domains

### Tables (Sequences)

TABLE A
Exemplary variants of domain of human CD86

| SEQ | | |
|---|---|---|
| ID | | |
| NO. | DESIGNATION | SEQUENCE |
| 6 | 900 | LKIQAYFNETADLPCQFANSQNQSLSELVVFWQDQENLVLNEVYL |
| | | GKEKFDSVDSKYMGRTSFDSDSWTLRLHNLQIKDKGIYQCVIHHK |
| | | KPSGLVKIHEMNSELSVLA |
| 7 | 901 | LKIQAYFNETADLPCQFANSQNLTLSELVVFWQDQENLVLNEVYLG |
| | | KEKFDSVHSKYMGRTSFDSDSWTLRLHNLQIKDKGIYQCVIHHKKP |
| | | TGMIKIHEMNSELSVLT |
| 8 | 904 | LKIQAYFNETADLPCQFANSQNQSLSELIVFWQDQENLVLNEVYLG |
| | | KERFDAVDSKYMGRTSFDSDSWTLRLHNLQIKDKGIYQCIIHHKKP |
| | | SGMVKIHQMDSELSVLA |
| 9 | 906 | LKIQAYINETADLPCQFANSQNLSLSELVVFWQDQENLVLNEVYLG |
| | | KERFDSVDSKYMGRTSFDSDSWTLRLHNLQIKDKGFYQCIIHHKKP |
| | | TGLVKIHEMNSELSVLA |
| 10 | 907 | LKIQAYFNETADLPCQFANSQNQSLSELVVFWQDQENLVLNEVYL |
| | | GKEKFDSVHSKYMGRTSFDSDSWTLRLHNLQIKDKGLYQCIIHHKK |
| | | PTGMIKIHEMNSELSVLA |
| 11 | 908 | LKIQAYFNETADLPCQFANSQNQSLSELVVFWQDQENLVLNEVYL |
| | | GKEKFDSVHSKYMGRTSFDSDSWTLRLHNLQIKDKGIYQCIIHHKK |
| | | PTGMVKIHEMNSELSVLA |
| 12 | 910 | LKIQAYFNETADLPCQFANSQNQSLSELVVFWQDQENLVLNEVYL |
| | | GKEKFDSVDSKYMGRTSFDSDSWTLRLHNLQIKDKGIYQCIIHHKK |
| | | PTGMVKIHEMNSELSVLA |
| 13 | 915 | LKIQAYFNETADLPCQFANSQNQSLSELVVFWQDQENLILNEVYLG |
| | | KEKFDSVDSKYMGRTSFDSDSWTLRLHNLQIKDKGFYQCIIHHKKP |
| | | SGLIKIHQMDSELSVLA |
| 14 | 938 | LKIQAYFNETADLPCQFANSQNQSLSELVVFWQDQENLILNEVYLG |
| | | KEKFDSVHSKYMGRTSFDSDSWTLRLHNLQIKDKGIYQCIIHHKKP |
| | | TGMVKIHQMNSELSVLA |
| 15 | 1038 | APLKIQAYFNETADLPCQFANSQNLSLSELVVFWQDQENLVLNEVY |
| | | LGKEKFDSVDSKYMGRTSFDSDSWTLRLHNLQIKDKGIYQCIIHHK |
| | | KPTGMVKIHEMNSELSVLA |
| 16 | 1039 | APLKIQAYFNETADLPCQFANSQNLSLSELVVFWQDQENLVLNEVY |
| | | LGKEKFDSVSSKYMGRTSFDSDSWTLRLHNLQIKDKGIYQCIIHHK |
| | | KPSGMVKIHQMDSELSVLA |
| 17 | 1040 | APLKIQAYFNETADLPCQFANSQNLSLSELVVFWQDQENLVLNEVY |
| | | LGKERFDSVDSKYMGRTSFDSDSWTLRLHNLQIKDKGRYQCIIHH |
| | | KKPTGMINIHQMNSELSVLA |
| 18 | 1041 | APLKIQAYLNETADLPCQFANSQNLSLSELVVFWQDQENLVLNEVY |
| | | LGKEKFDSVDSKYMGRTSFDSDSWTLRLHNLQIKDKGIYQCIIHHK |
| | | KPTGLVKIHEMNSELSVLA |
| 19 | 1042 | APLKIQAYFNETADLPCQFANSQNLSLSELVVFWQDQENLVLNEVY |
| | | LGKEIFDSVSSKYMGRTSFDSDSWTLRLHNLQIKDKGIYQCIIHHKK |
| | | PSGMVKIHQMDSELSVLA |
| 20 | 1043 | APLKIQAYFNETADLPCQFANSQNLSLSELVVFWQDQENLVLNEVY |
| | | LGKEKFDSVDSKYMGRTSFDSDSWTLRLHNLQIKDKGIYQCIIHHK |
| | | KPTGMIKIHEMNSELSVLA |
| 21 | 1044 | APLKIQAYFNETADLPCQFANSQNLTLSELVVFWQDQENLVLNEVY |
| | | LGKEKFDSVSSKYMGRTSFDSDSWTLRLHNLQIKDKGIYQCIIHHK |
| | | KPTGMIKIHEMSSELSVLA |
| 22 | 1045 | APLKIQAYFNETADLPCQFANSQNLTLSELVVFWQDQENLVLNEVY |
| | | LGKEKFDSVDSKYMGRTSFDSDSWTLRLHNLQIKDKGLYQCIIHHK |
| | | KPTGLVKIHEMNSELSVLA |
| 23 | 1046 | APLKIQAYFNETADLPCQFANSQNQSLSELVVFWQDQENLVLNEV |
| | | YLGKEKFDSVDSKYMGRTSFDSDSWTLRLHNLQIEDKGIYQCIIHH |
| | | KKPSGMVKIHQMDSELSVLA |
| 24 | 1047 | APLKIQAYFNETADLPCQFANSQNLSLSELVVFWQDQENLVLNEVY |
| | | LGKEKFDSVDSKYMGRTSFDSDSWTLRLHNLQIKDKGIYQCIIHHK |
| | | KPTGLVKIHEMNSELSVLA |

TABLE B
Exemplary polynucleotides encoding B2 - CTLA-4

| SEQ | | |
|---|---|---|
| ID | | |
| 25 | 900 | CTCAAAATCCAAGCGTACTTCAACGAAACTGCAGACTTACCGTG |
| | | TCAGTTTGCCAATTCGCAGAATCAAAGCCTGAGCGAACTGGTG |
| | | GTTTTCTGGCAGGATCAGGAGAACCTGGTTCTGAACGAAGTCTA |
| | | TCTGGGCAAAGAGAAATTCGACAGCGTGGACAGCAAGTATATG |
| | | GGCCGCACCAGCTTTGATAGCGACAGCTGGACCCTGCGTCTGC |
| | | ACAATCTGCAAATCAAAGATAAGGGTATCTACCAGTGCGTGATC |
| | | CACCATAAGAAGCCGAGCGGTCTGGTGAAGATTCACGAGATGA |
| | | ACTCCGAGTTGTCTGTCCTGGCG |
| 26 | 901 | CTCAAAATCCAAGCGTACTTCAACGAAACTGCAGACTTACCGTG |
| | | TCAGTTTGCCAATTCGCAGAATCTGACCCTGAGCGAACTGGTG |
| | | GTTTTCTGGCAGGATCAGGAGAACCTGGTTCTGAACGAAGTCTA |
| | | TCTGGGCAAAGAGAAATTCGACAGCGTGCATAGCAAGTATATG |
| | | GGCCGCACCAGCTTTGATAGCGACAGCTGGACCCTGCGTCTGC |
| | | ACAATCTGCAAATCAAAGATAAGGGTATCTACCAGTGCGTGATC |
| | | CACCATAAGAAGCCGACGGGTATGATTAAGATTCACGAGATGAA |
| | | CTCCGAGTTGTCTGTCCTGACC |
| 27 | 904 | CTCAAAATCCAAGCGTACTTCAACGAAACTGCAGACTTACCGTG |
| | | TCAGTTTGCCAATTCGCAGAATCAAAGCCTGAGCGAACTGATCG |
| | | TTTTCTGGCAGGATCAGGAGAACCTGGTTCTGAACGAAGTCTAT |
| | | CTGGGCAAAGAGCGGTTCGACGCCGTGGACAGCAAGTATATGG |
| | | GCCGCACCAGCTTTGATAGCGACAGCTGGACCCTGCGTCTGCA |
| | | CAATCTGCAAATCAAAGATAAGGGTATCTACCAGTGCATTATCC |
| | | ACCATAAGAAGCCGAGCGGTATGGTGAAGATTCACCAAATGGA |
| | | CTCCGAGTTGTCTGTCCTGGCG |
| 28 | 906 | CTCAAAATCCAAGCGTACATCAACGAAACTGCAGACTTACCGTG |
| | | TCAGTTTGCCAATTCGCAGAATCTGAGCCTGAGCGAACTGGTG |
| | | GTTTTCTGGCAGGATCAGGAGAACCTGGTTCTGAACGAAGTCTA |
| | | TCTGGGCAAAGAGCGGTTCGACAGCGTGGACAGCAAGTATATG |
| | | GGCCGCACCAGCTTTGATAGCGACAGCTGGACCCTGCGTCTGC |
| | | ACAATCTGCAAATCAAAGATAAGGGTTTCTACCAGTGCATTATC |
| | | CACCATAAGAAGCCGACGGGTCTGGTGAAGATTCACGAGATGA |
| | | ACTCCGAGTTGTCTGTCCTGGCG |
| 29 | 907 | CTCAAAATCCAAGCGTACTTCAACGAAACTGCAGACTTACCGTG |
| | | TCAGTTTGCCAATTCGCAGAATCAAAGCCTGAGCGAACTGGTG |
| | | GTTTTCTGGCAGGATCAGGAGAACCTGGTTCTGAACGAAGTCTA |
| | | TCTGGGCAAAGAGAAATTCGACAGCGTGCATAGCAAGTATATG |
| | | GGCCGCACCAGCTTTGATAGCGACAGCTGGACCCTGCGTCTGC |
| | | ACAATCTGCAAATCAAAGATAAGGGTCTGTACCAGTGCATTATC |
| | | CACCATAAGAAGCCGACGGGTATGATTAAGATTCACGAGATGAA |
| | | CTCCGAGTTGTCTGTCCTGGCG |
| 30 | 908 | CTCAAAATCCAAGCGTACTTCAACGAAACTGCAGACTTACCGTG |
| | | TCAGTTTGCCAATTCGCAGAATCAAAGCCTGAGCGAACTGGTG |
| | | GTTTTCTGGCAGGATCAGGAGAACCTGGTTCTGAACGAAGTCTA |
| | | TCTGGGCAAAGAGAAATTCGACAGCGTGCATAGCAAGTATATG |
| | | GGCCGCACCAGCTTTGATAGCGACAGCTGGACCCTGCGTCTGC |
| | | ACAATCTGCAAATCAAAGATAAGGGTATCTACCAGTGCATTATC |
| | | CACCATAAGAAGCCGACGGGTATGGTGAAGATTCACGAGATGA |
| | | ACTCCGAGTTGTCTGTCCTGGCG |
| 31 | 910 | CTCAAAATCCAAGCGTACTTCAACGAAACTGCAGACTTACCGTG |
| | | TCAGTTTGCCAATTCGCAGAATCAAAGCCTGAGCGAACTGGTG |
| | | GTTTTCTGGCAGGATCAGGAGAACCTGGTTCTGAACGAAGTCTA |
| | | TCTGGGCAAAGAGAAATTCGACAGCGTGGACAGCAAGTATATG |
| | | GGCCGCACCAGCTTTGATAGCGACAGCTGGACCCTGCGTCTGC |
| | | ACAATCTGCAAATCAAAGATAAGGGTATCTACCAGTGCATTATC |
| | | CACCATAAGAAGCCGACGGGTATGGTGAAGATTCACGAGATGA |
| | | ACTCCGAGTTGTCTGTCCTGGCG |
| 32 | 915 | CTCAAAATCCAAGCGTACTTCAACGAAACTGCAGACTTACCGTG |
| | | TCAGTTTGCCAATTCGCAGAATCAAAGCCTGAGCGAACTGGTG |
| | | GTTTTCTGGCAGGATCAGGAGAACCTGATCCTGAACGAAGTCTA |
| | | TCTGGGCAAAGAGAAATTCGACAGCGTGGACAGCAAGTATATG |
| | | GGCCGCACCAGCTTTGATAGCGACAGCTGGACCCTGCGTCTGC |
| | | ACAATCTGCAAATCAAAGATAAGGGTTTCTACCAGTGCATTATC |
| | | CACCATAAGAAGCCGAGCGGTCTGATTAAGATTCACCAAATGGA |
| | | CTCCGAGTTGTCTGTCCTGGCG |
| 33 | 938 | CTCAAAATCCAAGCGTACTTCAACGAAACTGCAGACTTACCGTG |
| | | TCAGTTTGCCAATTCGCAGAATCTGAGCCTGAGCGAACTGGTG |
| | | GTTTTCTGGCAGGATCAGGAGAACCTGATCCTGAACGAAGTCTA |
| | | TCTGGGCAAAGAGCGGTTCGACAGCGTGCATAGCAAGTATATG |
| | | GGCCGCACCAGCTTTGATAGCGACAGCTGGACCCTGCGTCTGC |
| | | ACAATCTGCAAATCAAAGATAAGGGTCTGTACCAGTGCATTATC |
| | | CACCATAAGAAGCCGAGCGGTATGGTGAAGATTCACGAGATGA |
| | | ACTCCGAGTTGTCTGTCCTGGCG |
| 34 | 1038 | GCCCCCCTCAAAATCCAAGCGTACTTCAACGAAACTGCAGACTT |
| | | ACCGTGTCAGTTTGCCAATTCGCAGAATCTGAGCCTGAGCGAA |
| | | CTGGTGGTTTTCTGGCAGGATCAGGAGAACCTGGTTCTGAACG |
| | | AAGTCTATCTGGGCAAAGAGAAATTCGACAGCGTGGACAGCAA |
| | | GTATATGGGCCGCACCAGCTTTGATAGCGACAGCTGGACCCTG |
| | | CGTCTGCACAATCTGCAAATCAAAGATAAGGGTATCTACCAGTG |
| | | CATTATCCACCATAAGAAGCCGACGGGTATGGTGAAGATTCAC |
| | | GAGATGAACTCCGAGTTGTCTGTCCTGGCG |
| 35 | 1039 | GCCCCCCTCAAAATCCAAGCGTACTTCAACGAAACTGCAGACTT |
| | | ACCGTGTCAGTTTGCCAATTCGCAGAATCTGAGCCTGAGCGAA |
| | | CTGGTGGTTTTCTGGCAGGATCAGGAGAACCTGGTTCTGAACG |
| | | AAGTCTATCTGGGCAAAGAGAAATTCGACAGCGTGAGTAGCAA |
| | | GTATATGGGCCGCACCAGCTTTGATAGCGACAGCTGGACCCTG |
| | | CGTCTGCACAATCTGCAAATCAAAGATAAGGGTATCTACCAGTG |
| | | CATTATCCACCATAAGAAGCCGAGCGGTATGGTGAAGATTCACC |
| | | AAATGGACTCCGAGTTGTCTGTCCTGGCG |
| 36 | 1040 | GCCCCCCTCAAAATCCAAGCGTACTTCAACGAAACTGCAGACTT |
| | | ACCGTGTCAGTTTGCCAATTCGCAGAATCTGAGCCTGAGCGAA |
| | | CTGGTGGTTTTCTGGCAGGATCAGGAGAACCTGGTTCTGAACG |
| | | AAGTCTATCTGGGCAAAGAGCGGTTCGACAGCGTGGACAGCAA |
| | | GTATATGGGCCGCACCAGCTTTGATAGCGACAGCTGGACCCTG |
| | | CGTCTGCACAATCTGCAAATCAAAGATAAGGGTAGGTACCAGTG |
| | | CATTATCCACCATAAGAAGCCGACGGGTATGATTAATATTCACC |
| | | AAATGAACTCCGAGTTGTCTGTCCTGGCG |
| 37 | 1041 | GCCCCCCTCAAAATCCAAGCGTACCTCAACGAAACTGCAGACTT |
| | | ACCGTGTCAGTTTGCCAATTCGCAGAATCTGAGCCTGAGCGAA |
| | | CTGGTGGTTTTCTGGCAGGATCAGGAGAACCTGGTTCTGAACG |
| | | AAGTCTATCTGGGCAAAGAGAAATTCGACAGCGTGGACAGCAA |
| | | GTATATGGGCCGCACCAGCTTTGATAGCGACAGCTGGACCCTG |
| | | CGTCTGCACAATCTGCAAATCAAAGATAAGGGTATCTACCAGTG |
| | | CATTATCCACCATAAGAAGCCGACGGGTCTGGTGAAGATTCAC |
| | | GAGATGAACTCCGAGTTGTCTGTCCTGGCG |
| 38 | 1042 | GCCCCCCTCAAAATCCAAGCGTACTTCAACGAAACTGCAGACTT |
| | | ACCGTGTCAGTTTGCCAATTCGCAGAATCTGAGCCTGAGCGAA |
| | | CTGGTGGTTTTCTGGCAGGATCAGGAGAACCTGGTTCTGAACG |
| | | AAGTCTATCTGGGCAAAGAGATTTTCGACAGCGTGAGTAGCAA |
| | | GTATATGGGCCGCACCAGCTTTGATAGTGACAGCTGGACCCTG |
| | | CGTCTGCACAATCTGCAAATCAAAGATAAGGGTATCTACCAGTG |
| | | CATTATCCACCATAAGAAGCCGAGCGGTATGGTGAAGATTCACC |
| | | AAATGGACTCCGAGTTGTCTGTCCTGGCG |
| 39 | 1043 | GCCCCCCTCAAAATCCAAGCGTACTTCAACGAAACTGCAGACTT |
| | | ACCGTGTCAGTTTGCCAATTCGCAGAATCTGAGCCTGAGCGAA |
| | | CTGGTGGTTTTCTGGCAGGATCAGGAGAACCTGGTTCTGAACG |
| | | AAGTCTATCTGGGCAAAGAGAAATTCGACAGCGTGGATAGCAA |
| | | GTATATGGGCCGCACCAGCTTTGATAGCGACAGCTGGACCCTG |
| | | CGTCTGCACAATCTGCAAATCAAAGATAAGGGTATCTACCAGTG |
| | | CATTATCCACCATAAGAAGCCGACGGGTATGATTAAGATTCACG |
| | | AGATGAACTCCGAGTTGTCTGTCCTGGCG |
| 40 | 1044 | GCCCCCCTCAAAATCCAAGCGTACTTCAACGAAACTGCAGACTT |
| | | ACCGTGTCAGTTTGCCAATTCGCAGAATCTGACCCTGAGCGAA |
| | | CTGGTGGTTTTCTGGCAGGATCAGGAGAACCTGGTTCTGAACG |
| | | AAGTCTATCTGGGCAAAGAGAAATTCGACAGCGTGTCTAGCAA |
| | | GTATATGGGCCGCACCAGCTTTGATAGCGACAGCTGGACCCTG |
| | | CGTCTGCACAATCTGCAAATCAAAGATAAGGGTATCTACCAGTG |
| | | CATTATCCACCATAAGAAGCCGACGGGTATGATTAAGATTCACG |
| | | AGATGAGCTCCGAGTTGTCTGTCCTGGCG |
| 41 | 1045 | GCCCCCCTCAAAATCCAAGCGTACTTCAACGAAACTGCAGACTT |
| | | ACCGTGTCAGTTTGCCAATTCGCAGAATCTGACCCTGAGCGAA |
| | | CTGGTGGTTTTCTGGCAGGATCAGGAGAACCTGGTTCTGAACG |
| | | AAGTCTATCTGGGCAAAGAGAAATTCGACAGCGTGGACAGCAA |
| | | GTATATGGGCCGCACCAGCTTTGATAGCGACAGCTGGACCCTG |
| | | CGTCTGCACAATCTGCAAATCAAAGATAAGGGTCTGTACCAGTG |
| | | CATTATCCACCATAAGAAGCCGACGGGTCTGGTGAAGATTCAC |
| | | GAGATGAACTCCGAGTTGTCTGTCCTGGCG |
| 42 | 1046 | GCCCCCCTCAAAATCCAAGCGTACTTCAACGAAACTGCAGACTT |
| | | ACCGTGTCAGTTTGCCAATTCGCAGAATCAAAGCCTGAGCGAA |
| | | CTGGTGGTTTTCTGGCAGGATCAGGAGAACCTGGTTCTGAACG |
| | | AAGTCTATCTGGGCAAAGAGAAATTCGACAGCGTGGACAGCAA |
| | | GTATATGGGCCGCACCAGCTTTGATAGCGACAGCTGGACCCTG |
| | | CGTCTGCACAATCTGCAAATCGAAGATAAGGGTATCTACCAGTG |
| | | CATTATCCACCATAAGAAGCCGAGCGGTATGGTGAAGATTCACC |
| | | AAATGGACTCCGAGTTGTCTGTCCTGGCG |
| 43 | 1047 | GCCCCCCTCAAAATCCAAGCGTACTTCAACGAAACTGCAGACTT |
| | | ACCGTGTCAGTTTGCCAATTCGCAGAATCTGAGCCTGAGCGAA |
| | | CTGGTGGTTTTCTGGCAGGATCAGGAGAACCTGGTTCTGAACG |
| | | AAGTCTATCTGGGCAAAGAGAAATTCGACAGCGTGGACAGCAA |
| | | GTATATGGGCCGCACCAGCTTTGATAGCGACAGCTGGACCCTG |
| | | CGTCTGCACAATCTGCAAATCAAAGATAAGGGTATCTACCAGTG |
| | | CATTATCCACCATAAGAAGCCGACGGGTCTGGTGAAGATTCAC |
| | | GAGATGAACTCCGAGTTGTCTGTCCTGGCG |

TABLE C
Exemplary sequences

| SEQ | | | |
|---|---|---|---|
| ID | | | |
| NO. | CHAIN NO. | TYPE | SEQUENCE |
| 52 | 2348, heavy | aa | EVQLLESGGGLVQPGGSLRLSCAASGFTFGYYYMSW |
| | chain VH | | VRQAPGKGLEWVSGISSPSSYTYYADSVKGRFTISRD |
| | | | NSKNTLYLQMNSLRAEDTAVYYCARYYGSYFDYWGQ |
| | | | GTLVTVSS |
| 53 | 2348, heavy | nt | GAGGTGCAGCTGTTGGAGAGCGGGGGAGGCTTGGT |
| | chain VH | | ACAGCCTGGGGGGTCCCTGCGCCTCTCCTGTGCAG |
| | | | CCAGCGGATTCACCTTTGGTTACTACTACATGTCTTG |
| | | | GGTCCGCCAGGCTCCAGGGAAGGGGCTGGAGTGG |
| | | | GTCTCAGGTATTTCTTCTCCTTCTTCTTACACATACTA |
| | | | TGCAGACTCCGTGAAGGGCCGGTTCACCATCTCCC |
| | | | GTGACAATTCCAAGAACACGCTGTATCTGCAAATGA |
| | | | ACAGCCTGCGTGCCGAGGACACGGCTGTATATTATT |
| | | | GTGCGCGCTACTACGGTTCTTACTTTGACTATTGGG |
| | | | GCCAGGGAACCCTGGTCACCGTCTCCTCA |
| 54 | 2372 (VH) | aa | EVQLLESGGGLVQPGGSLRLSCAASGFTFSGYSMGW |
| | | | VRQAPGKGLEWVSAISGSGGSTYYADSVKGRFTISRD |
| | | | NSKNTLYLQMNSLRAEDTAVYYCARYPWGYYFDYWG |
| | | | QGTLVTVSS |
| 55 | 2372 (VH) | nt | GAGGTGCAGCTGTTGGAGAGCGGGGGAGGCTTGGT |
| | | | ACAGCCTGGGGGGTCCCTGCGCCTCTCCTGTGCAG |
| | | | CCAGCGGATTCACCTTTTCTGGTTACTCTATGGGTTG |
| | | | GGTCCGCCAGGCTCCAGGGAAGGGGCTGGAGTGG |
| | | | GTCTCAGCTATTAGTGGTAGTGGTGGTAGCACATAC |
| | | | TATGCAGACTCCGTGAAGGGCCGGTTCACCATCTCC |
| | | | CGTGACAATTCCAAGAACACGCTGTATCTGCAAATG |
| | | | AACAGCCTGCGTGCCGAGGACACGGCTGTATATTAT |
| | | | TGTGCGCGCTACCCGTGGGGTTACTACTTTGACTAT |
| | | | TGGGGCCAGGGAACCCTGGTCACCGTCTCCTCA |
| 56 | 2396 (VH) | aa | EVQLLESGGGLVQPGGSLRLSCAASGFTFSSYAMSWV |
| | | | RQAPGKGLEWVSAISGSGGSTYYADSVKGRFTISRDN |
| | | | SKNTLYLQMNSLRAEDTAVYYCARAYPVHGYWVFDY |
| | | | WGQGTLVTVSS |
| 57 | 2396 (VH) | nt | GAGGTGCAGCTGTTGGAGAGCGGGGGAGGCTTGGT |
| | | | ACAGCCTGGGGGGTCCCTGCGCCTCTCCTGTGCAG |
| | | | CCAGCGGATTCACCTTTAGCAGCTATGCCATGAGCT |
| | | | GGGTCCGCCAGGCTCCAGGGAAGGGGCTGGAGTG |
| | | | GGTCTCAGCTATTAGTGGTAGTGGTGGTAGCACATA |
| | | | CTATGCAGACTCCGTGAAGGGCCGGTTCACCATCTC |
| | | | CCGTGACAATTCCAAGAACACGCTGTATCTGCAAAT |
| | | | GAACAGCCTGCGTGCCGAGGACACGGCTGTATATTA |
| | | | TTGTGCGCGCGCTTACCCGGTTCATGGTTACTGGGT |
| | | | TTTTGACTATTGGGGCCAGGGAACCCTGGTCACCGT |
| | | | CTCCTCA |
| 58 | 2404 (VH) | aa | EVQLLESGGGLVQPGGSLRLSCAASGFTFSYSSMSWV |
| | | | RQAPGKGLEWVSYIGSGGSHTYYADSVKGRFTISRDN |
| | | | SKNTLYLQMNSLRAEDTAVYYCARYSYYFDYWGQGTL |
| | | | VTVSS |
| 59 | 2404 (VH) | nt | GAGGTGCAGCTGTTGGAGAGCGGGGGAGGCTTGGT |
| | | | ACAGCCTGGGGGGTCCCTGCGCCTCTCCTGTGCAG |
| | | | CCAGCGGATTCACCTTTTCTTACTCTTCTATGTCTTG |
| | | | GGTCCGCCAGGCTCCAGGGAAGGGGCTGGAGTGG |
| | | | GTCTCATACATTGGTTCTGGTGGTTCTCACACATACT |
| | | | ATGCAGACTCCGTGAAGGGCCGGTTCACCATCTCCC |
| | | | GTGACAATTCCAAGAACACGCTGTATCTGCAAATGA |
| | | | ACAGCCTGCGTGCCGAGGACACGGCTGTATATTATT |
| | | | GTGCGCGCTACTCTTACTACTTTGACTATTGGGGCC |
| | | | AGGGAACCCTGGTCACCGTCTCCTCA |
| 60 | 2349 (VL)(mAb- | nt | GACATCCAGATGACCCAGTCTCCATCCTCCCTGAGC |
| | without CTLA- | | GCATCTGTAGGAGACCGCGTCACCATCACTTGCCGG |
| | 4 binding | | GCAAGTCAGGCTATTAGCGCTTATTTAAATTGGTATC |
| | domain) | | AGCAGAAACCAGGGAAAGCCCCTAAGCTCCTGATCT |
| | | | ATGCTGCATCCAGTTTGCAAAGTGGGGTCCCATCAC |
| | | | GTTTCAGTGGCAGTGGAAGCGGGACAGATTTCACTC |
| | | | TCACCATCAGCAGTCTGCAACCTGAAGATTTTGCAA |
| | | | CTTATTACTGTCAACAGTCTTACGGTTACTACCTGTA |
| | | | CACTTTTGGCCAGGGGACCAAGCTGGAGATCAAACG |
| | | | T |
| 61 | 2349 (VL)(mAb- | aa | DIQMTQSPSSLSASVGDRVTITCRASQAISAYLNWYQQ |
| | without CTLA- | | KPGKAPKLLIYAASSLQSGVPSRFSGSGSGTDFTLTISS |
| | 4 binding | | LQPEDFATYYCQQSYGYYLYTFGQGTKLEIK |
| | domain) | | |
| 62 | 2373 (VL)(mAb- | nt | GACATCCAGATGACCCAGTCTCCATCCTCCCTGAGC |
| | without CTLA- | | GCATCTGTAGGAGACCGCGTCACCATCACTTGCCGG |
| | 4 binding | | GCAAGTCAGGGTATTAGAGCTTATTTAAATTGGTATC |
| | domain) | | AGCAGAAACCAGGGAAAGCCCCTAAGCTCCTGATCT |
| | | | ATGCTGTATCCAGTTTGCAAAGTGGGGTCCCATCAC |
| | | | GTTTCAGTGGCAGTGGAAGCGGGACAGATTTCACTC |
| | | | TCACCATCAGCAGTCTGCAACCTGAAGATTTTGCAA |
| | | | CTTATTACTGTCAACAGTACTACTACCCGCCGCTGTC |
| | | | CACTTTTGGCCAGGGGACCAAGCTGGAGATCAAACG |
| | | | T |
| 63 | 2373 (VL)(mAb- | aa | DIQMTQSPSSLSASVGDRVTITCRASQGIRAYLNWYQQ |
| | without CTLA- | | KPGKAPKLLIYAVSSLQSGVPSRFSGSGSGTDFTLTISS |
| | 4 binding | | LQPEDFATYYCQQYYYPPLSTFGQGTKLEIK |
| | domain) | | |
| 64 | 2397 (VL)(mAb- | nt | GACATCCAGATGACCCAGTCTCCATCCTCCCTGAGC |
| | without CTLA- | | GCATCTGTAGGAGACCGCGTCACCATCACTTGCCGG |
| | 4 binding | | GCAAGTCAGAGCATTAGCAGCTATTTAAATTGGTATC |
| | domain) | | AGCAGAAACCAGGGAAAGCCCCTAAGCTCCTGATCT |
| | | | ATGCTGCATCCAGTTTGCAAAGTGGGGTCCCATCAC |
| | | | GTTTCAGTGGCAGTGGAAGCGGGACAGATTTCACTC |
| | | | TCACCATCAGCAGTCTGCAACCTGAAGATTTTGCAA |
| | | | CTTATTACTGTCAACAGTCTGTTTCTACTCCGCCCAC |
| | | | TTTTGGCCAGGGGACCAAGCTGGAGATCAAACGT |
| 65 | 2397 (VL)(mAb- | aa | DIQMTQSPSSLSASVGDRVTITCRASQSISSYLNWYQQ |
| | without CTLA- | | KPGKAPKLLIYAASSLQSGVPSRFSGSGSGTDFTLTISS |
| | 4 binding | | LQPEDFATYYCQQSVSTPPTFGQGTKLEIK |
| | domain) | | |
| 66 | 2405 (VL)(mAb- | nt | GACATCCAGATGACCCAGTCTCCATCCTCCCTGAGC |
| | without CTLA- | | GCATCTGTAGGAGACCGCGTCACCATCACTTGCCGG |
| | 4 binding | | GCAAGTCAGAGCATTAGCAGCTATTTAAATTGGTATC |
| | domain) | | AGCAGAAACCAGGGAAAGCCCCTAAGCTCCTGATCT |
| | | | ATGCTGCATCCAGTTTGCAAAGTGGGGTCCCATCAC |
| | | | GTTTCAGTGGCAGTGGAAGCGGGACAGATTTCACTC |
| | | | TCACCATCAGCAGTCTGCAACCTGAAGATTTTGCAA |
| | | | CTTATTACTGTCAACAGAGTCATTACTGGTACCCGCT |
| | | | CACTTTTGGCCAGGGGACCAAGCTGGAGATCAAACG |
| | | | T |
| 67 | 2405 (VL)(mAb- | aa | DIQMTQSPSSLSASVGDRVTITCRASQSISSYLNWYQQ |
| | without CTLA- | | KPGKAPKLLIYAASSLQSGVPSRFSGSGSGTDFTLTISS |
| | 4 binding | | LQPEDFATYYCQQSHYWYPLTFGQGTKLEIK |
| | domain) | | |
| 68 | 2349 | nt | GACATCCAGATGACCCAGTCTCCATCCTCCCTGAGC |
| | Light chain VL, | | GCATCTGTAGGAGACCGCGTCACCATCACTTGCCGG |
| | with constant | | GCAAGTCAGGCTATTAGCGCTTATTTAAATTGGTATC |
| | kappa | | AGCAGAAACCAGGGAAAGCCCCTAAGCTCCTGATCT |
| | sequence, linker | | ATGCTGCATCCAGTTTGCAAAGTGGGGTCCCATCAC |
| | and CD86 | | GTTTCAGTGGCAGTGGAAGCGGGACAGATTTCACTC |
| | mutant 1040 | | TCACCATCAGCAGTCTGCAACCTGAAGATTTTGCAA |
| | inclusive intron | | CTTATTACTGTCAACAGTCTTACGGTTACTACCTGTA |
| | sequence | | CACTtTTGGCCAGGGGACCAAGCTGGAGATCAAACG |
| | | | Tgagtcgtacgctagcaagcttgatatcgaattctaaactctgagggggtcggatgac |
| | | | gtggccattctttgcctaaagcattgagtttactgcaaggtcagaaaagcatgcaaagc |
| | | | cctcagaatggctgcaaagagctccaacaaaacaatttagaactttattaaggaatag |
| | | | ggggaagctaggaagaaactcaaaacatcaagattttaaatacgcttcttggtctcctt |
| | | | gctataattatctgggataagcatgctgttttctgtctgtccctaacatgccctgtgattat |
| | | | ccgcaaacaacacacccaagggcagaactttgttacttaaacaccatcctgtttgcttc |
| | | | tttcctcagGAACTGTGGCTGCACCATCTGTCTTCATCTTCCCGCCATC |
| | | | TGATGAGCAGTTGAAATCTGGAACTGCCTCTGTTGTGTGCCTGCT |
| | | | GAATAACTTCTATCCCAGAGAGGCCAAAGTACAGTGGAAGGTGG |
| | | | ATAACGCCCTCCAATCGGGTAACTCCCAGGAGAGTGTCACAGAGC |
| | | | AGGACAGCAAGGACAGCACCTACAGCCTCAGCAGCACCCTGACG |
| | | | CTGAGCAAAGCAGACTACGAGAAACACAAAGTCTACGCCTGCGA |
| | | | AGTCACCCATCAGGGCCTGAGCTCGCCCGTCACAAAGAGCTTCAA |
| | | | CAGGGGAGAGTGTAGCGGAGGAGGAGGAAGCGGAGGAGGAGG |
| | | | AAGCGCCCCCCTCAAAATCCAAGCGTACTTCAACGAAACTGCAGA |
| | | | CTTACCGTGTCAGTTTGCCAATTCGCAGAATCTGAGCCTGAGCGA |
| | | | ACTGGTGGTTTTCTGGCAGGATCAGGAGAACCTGGTTCTGAACGA |
| | | | AGTCTATCTGGGCAAAGAGCGGTTCGACAGCGTGGACAGCAAGT |
| | | | ATATGGGCCGCACCAGCTTTGATAGCGACAGCTGGACCCTGCGTC |
| | | | TGCACAATCTGCAAATCAAAGATAAGGGTAGGTACCAGTGCATTA |
| | | | TCCACCATAAGAAGCCGACGGGTATGATTAATATTCACCAAATGA |
| | | | ACTCCGAGTTGTCTGTCCTGGCG |
| 69 | 2349 light chain | aa | DIQMTQSPSSLSASVGDRVTITCRASQAISAYLNWYQQ |
| | VL, with | | KPGKAPKLLIYAASSLQSGVPSRFSGSGSGTDFTLTISS |
| | constant kappa, | | LQPEDFATYYCQQSYGYYLYTFGQGTKLEIK |
| | sequence linker | | RTVAAPSVFIFPPSDEQLKSGTASVVCLLNNFYPREAKVQWKV |
| | (underlined) | | DNALQSGNSQESVTEQDSKDSTYSLSSTLTLSKADYEKHKVYAC |
| | and CD86 | | EVTHQGLSSPVTKSFNRGECSGGGGSGGGGSAPLKIQAYFNET |
| | mutant 1040 | | ADLPCQFANSQNLSLSELVVFWQDQENLVLNEVYLGKERFDS |
| | | | VDSKYMGRTSFDSDSWTLRLHNLQIKDKGRYQCIIHHKKPTG |
| | | | MINIHQMNSELSVLA |
| | | | LIGHT CHAIN PREFERABLY ASSEMBLES WITH |
| | | | A HEAVY CHAIN COMPRISING THE 2348 VH |
| | | | SEQUENCE |
| | | | THUS, COMPLETE MOLECULE MAY BE |
| | | | DESIGNATED 2348/2349 |
| 70 | 2373 Light chain | nt | GACATCCAGATGACCCAGTCTCCATCCTCCCTGAGC |
| | VL, with | | GCATCTGTAGGAGACCGCGTCACCATCACTTGCCGG |
| | constant kappa | | GCAAGTCAGGGTATTAGAGCTTATTTAAATTGGTATC |
| | sequence, linker | | AGCAGAAACCAGGGAAAGCCCCTAAGCTCCTGATCT |
| | and CD86 | | ATGCTGTATCCAGTTTGCAAAGTGGGGTCCCATCAC |
| | mutant 1040 | | GTTTCAGTGGCAGTGGAAGCGGGACAGATTTCACTC |
| | | | TCACCATCAGCAGTCTGCAACCTGAAGATTTTGCAA |
| | | | CTTATTACTGTCAACAGTACTACTACCCGCCGCTGTC |
| | | | CACTTTTGGCCAGGGGACCAAGCTGGAGATCAAACG |
| | | | Tgagtcgtacgctagcaagcttgatatcgaattctaaactctgagggggtcggatgac |
| | | | gtggccattctttgcctaaagcattgagtttactgcaaggtcagaaaagcatgcaaagc |
| | | | cctcagaatggctgcaaagagctccaacaaaacaatttagaactttattaaggaatag |
| | | | ggggaagctaggaagaaactcaaaacatcaagattttaaatacgcttcttggtctcctt |
| | | | gctataattatctgggataagcatgctgttttctgtctgtccctaacatgccctgtgattat |
| | | | ccgcaaacaacacacccaagggcagaactttgttacttaaacaccatcctgtttgcttc |
| | | | tttcctcagGAACTGTGGCTGCACCATCTGTCTTCATCTTCCCGCCATC |
| | | | TGATGAGCAGTTGAAATCTGGAACTGCCTCTGTTGTGTGCCTGCT |
| | | | GAATAACTTCTATCCCAGAGAGGCCAAAGTACAGTGGAAGGTGG |
| | | | ATAACGCCCTCCAATCGGGTAACTCCCAGGAGAGTGTCACAGAGC |
| | | | AGGACAGCAAGGACAGCACCTACAGCCTCAGCAGCACCCTGACG |
| | | | CTGAGCAAAGCAGACTACGAGAAACACAAAGTCTACGCCTGCGA |
| | | | AGTCACCCATCAGGGCCTGAGCTCGCCCGTCACAAAGAGCTTCAA |
| | | | CAGGGGAGAGTGTAGCGGAGGAGGAGGAAGCGGAGGAGGAGG |
| | | | AAGCGCCCCCCTCAAAATCCAAGCGTACTTCAACGAAACTGCAGA |
| | | | CTTACCGTGTCAGTTTGCCAATTCGCAGAATCTGAGCCTGAGCGA |
| | | | ACTGGTGGTTTTCTGGCAGGATCAGGAGAACCTGGTTCTGAACGA |
| | | | AGTCTATCTGGGCAAAGAGCGGTTCGACAGCGTGGACAGCAAGT |
| | | | ATATGGGCCGCACCAGCTTTGATAGCGACAGCTGGACCCTGCGTC |
| | | | TGCACAATCTGCAAATCAAAGATAAGGGTAGGTACCAGTGCATTA |
| | | | TCCACCATAAGAAGCCGACGGGTATGATTAATATTCACCAAATGA |
| | | | ACTCCGAGTTGTCTGTCCTGGCG |
| 71 | 2373 Light chain | aa | DIQMTQSPSSLSASVGDRVTITCRASQGIRAYLNWYQQ |
| | VL, with | | KPGKAPKLLIYAVSSLQSGVPSRFSGSGSGTDFTLTISS |
| | constant kappa | | LQPEDFATYYCQQYYYPPLSTFGQGTKLEIK |
| | sequence, linker | | RTVAAPSVFIFPPSDEQLKSGTASVVCLLNNFYPREAKVQWKV |
| | (underlined) | | DNALQSGNSQESVTEQDSKDSTYSLSSTLTLSKADYEKHKVYAC |
| | and CD86 | | EVTHQGLSSPVTKSFNRGECSGGGGSGGGGSAPLKIQAYFNET |
| | mutant 1040 | | ADLPCQFANSQNLSLSELVVFWQDQENLVLNEVYLGKERFDS |
| | | | VDSKYMGRTSFDSDSWTLRLHNLQIKDKGRYQCIIHHKKPTG |
| | | | MINIHQMNSELSVLA |
| | | | LIGHT CHAIN PREFERABLY ASSEMBLES WITH |
| | | | A HEAVY CHAIN COMPRISING THE 2372 VH |
| | | | SEQUENCE |
| | | | THUS, COMPLETE MOLECULE MAY BE |
| | | | DESIGNATED 2372/2373 |
| 72 | 2397 Light chain | nt | GACATCCAGATGACCCAGTCTCCATCCTCCCTGAGC |
| | VL, with | | GCATCTGTAGGAGACCGCGTCACCATCACTTGCCGG |
| | constant kappa | | GCAAGTCAGAGCATTAGCAGCTATTTAAATTGGTATC |
| | sequence, linker | | AGCAGAAACCAGGGAAAGCCCCTAAGCTCCTGATCT |
| | and CD86 | | ATGCTGCATCCAGTTTGCAAAGTGGGGTCCCATCAC |
| | mutant 1040 | | GTTTCAGTGGCAGTGGAAGCGGGACAGATTTCACTC |
| | | | TCACCATCAGCAGTCTGCAACCTGAAGATTTTGCAA |
| | | | CTTATTACTGTCAACAGTCTGTTTCTACTCCGCCCAC |
| | | | TTTTGGCCAGGGGACCAAGCTGGAGATCAAACGTgagt |
| | | | cgtacgctagcaagcttgatatcgaattctaaactctgagggggtcggatgacgtggcc |
| | | | attctttgcctaaagcattgagtttactgcaaggtcagaaaagcatgcaaagccctcag |
| | | | aatggctgcaaagagctccaacaaaacaatttagaactttattaaggaataggggga |
| | | | agctaggaagaaactcaaaacatcaagattttaaatacgcttcttggtctccttgctata |
| | | | attatctgggataagcatgctgttttctgtctgtccctaacatgccctgtgattatccgca |
| | | | aacaacacacccaagggcagaactttgttacttaaacaccatcctgtttgcttctttcct |
| | | | cagGAACTGTGGCTGCACCATCTGTCTTCATCTTCCCGCCATCTGAT |
| | | | GAGCAGTTGAAATCTGGAACTGCCTCTGTTGTGTGCCTGCTGAAT |
| | | | AACTTCTATCCCAGAGAGGCCAAAGTACAGTGGAAGGTGGATAA |
| | | | CGCCCTCCAATCGGGTAACTCCCAGGAGAGTGTCACAGAGCAGG |
| | | | ACAGCAAGGACAGCACCTACAGCCTCAGCAGCACCCTGACGCTGA |
| | | | GCAAAGCAGACTACGAGAAACACAAAGTCTACGCCTGCGAAGTC |
| | | | ACCCATCAGGGCCTGAGCTCGCCCGTCACAAAGAGCTTCAACAGG |
| | | | GGAGAGTGTAGCGGAGGAGGAGGAAGCGGAGGAGGAGGAAGC |
| | | | GCCCCCCTCAAAATCCAAGCGTACTTCAACGAAACTGCAGACTTAC |
| | | | CGTGTCAGTTTGCCAATTCGCAGAATCTGAGCCTGAGCGAACTGG |
| | | | TGGTTTTCTGGCAGGATCAGGAGAACCTGGTTCTGAACGAAGTCT |
| | | | ATCTGGGCAAAGAGCGETTCGACAGCGTGGACAGCAAGTATATG |
| | | | GGCCGCACCAGCTTTGATAGCGACAGCTGGACCCTGCGTCTGCAC |
| | | | AATCTGCAAATCAAAGATAAGGGTAGGTACCAGTGCATTATCCAC |
| | | | CATAAGAAGCCGACGGGTATGATTAATATTCACCAAATGAACTCC |
| | | | GAGTTGTCTGTCCTGGCG |
| 73 | 2397 Light chain | aa | DIQMTQSPSSLSASVGDRVTITCRASQSISSYLNWYQQ |
| | VL, with | | KPGKAPKLLIYAASSLQSGVPSRFSGSGSGTDFTLTISS |
| | constant kappa | | LQPEDFATYYCQQSVSTPPTFGQGTKLEIK |
| | sequence, linker | | RTVAAPSVFIFPPSDEQLKSGTASVVCLLNNFYPREAKVQWKV |
| | (underlined) | | DNALQSGNSQESVTEQDSKDSTYSLSSTLTLSKADYEKHKVYAC |
| | and CD86 | | EVTHQGLSSPVTKSFNRGECSGGGGSGGGGSAPLKIQAYFNET |
| | mutant 1040 | | ADLPCQFANSQNLSLSELVVFWQDQENLVLNEVYLGKERFDS |
| | | | VDSKYMGRTSFDSDSWTLRLHNLQIKDKGRYQCIIHHKKPTG |
| | | | MINIHQMNSELSVLA |
| | | | LIGHT CHAIN PREFERABLY ASSEMBLES WITH |
| | | | A HEAVY CHAIN COMPRISING THE 2396 VH |
| | | | SEQUENCE |
| | | | THUS, COMPLETE MOLECULE MAY BE |
| | | | DESIGNATED 2396/2397 |
| 74 | 2405 | nt | GACATCCAGATGACCCAGTCTCCATCCTCCCTGAGC |
| | Light chain VL, | | GCATCTGTAGGAGACCGCGTCACCATCACTTGCCGG |
| | with constant | | GCAAGTCAGAGCATTAGCAGCTATTTAAATTGGTATC |
| | kappa | | AGCAGAAACCAGGGAAAGCCCCTAAGCTCCTGATCT |
| | sequence, linker | | ATGCTGCATCCAGTTTGCAAAGTGGGGTCCCATCAC |
| | (underlined) | | GTTTCAGTGGCAGTGGAAGCGGGACAGATTTCACTC |
| | and CD86 | | TCACCATCAGCAGTCTGCAACCTGAAGATTTTGCAA |
| | mutant 1040 | | CTTATTACTGTCAACAGAGTCATTACTGGTACCCGCT |
| | | | CACTTTTGGCCAGGGGACCAAGCTGGAGATCAAACG |
| | | | Tgagtcgtacgctagcaagcttgatatcgaattctaaactctgagggggtcggatgac |
| | | | gtggccattctttgcctaaagcattgagtttactgcaaggtcagaaaagcatgcaaagc |
| | | | cctcagaatggctgcaaagagctccaacaaaacaatttagaactttattaaggaatag |
| | | | ggggaagctaggaagaaactcaaaacatcaagattttaaatacgcttcttggtctcctt |
| | | | gctataattatctgggataagcatgctgttttctgtctgtccctaacatgccctgtgattat |
| | | | ccgcaaacaacacacccaagggcagaactttgttacttaaacaccatcctgtttgcttc |
| | | | tttcctcagGAACTGTGGCTGCACCATCTGTCTTCATCTTCCCGCCATC |
| | | | TGATGAGCAGTTGAAATCTGGAACTGCCTCTGTTGTGTGCCTGCT |
| | | | GAATAACTTCTATCCCAGAGAGGCCAAAGTACAGTGGAAGGTGG |
| | | | ATAACGCCCTCCAATCGGGTAACTCCCAGGAGAGTGTCACAGAGC |
| | | | AGGACAGCAAGGACAGCACCTACAGCCTCAGCAGCACCCTGACG |
| | | | CTGAGCAAAGCAGACTACGAGAAACACAAAGTCTACGCCTGCGA |
| | | | AGTCACCCATCAGGGCCTGAGCTCGCCCGTCACAAAGAGCTTCAA |
| | | | CAGGGGAGAGTGTAGCGGAGGAGGAGGAAGCGGAGGAGGAGG |
| | | | AAGCGCCCCCCTCAAAATCCAAGCGTACTTCAACGAAACTGCAGA |
| | | | CTTACCGTGTCAGTTTGCCAATTCGCAGAATCTGAGCCTGAGCGA |
| | | | ACTGGTGGTTTTCTGGCAGGATCAGGAGAACCTGGTTCTGAACGA |
| | | | AGTCTATCTGGGCAAAGAGCGGTTCGACAGCGTGGACAGCAAGT |
| | | | ATATGGGCCGCACCAGCTTTGATAGCGACAGCTGGACCCTGCGTC |
| | | | TGCACAATCTGCAAATCAAAGATAAGGGTAGGTACCAGTGCATTA |
| | | | TCCACCATAAGAAGCCGACGGGTATGATTAATATTCACCAAATGA |
| | | | ACTCCGAGTTGTCTGTCCTGGCG |
| 75 | 2405 | aa | DIQMTQSPSSLSASVGDRVTITCRASQSISSYLNWYQQ |
| | Light chain VL, | | KPGKAPKLLIYAASSLQSGVPSRFSGSGSGTDFTLTISS |
| | with constant | | LQPEDFATYYCQQSHYWYPLTFGQGTKLEIK |
| | kappa | | RTVAAPSVFIFPPSDEQLKSGTASVVCLLNNFYPREAKVQWKV |
| | sequence, linker | | DNALQSGNSQESVTEQDSKDSTYSLSSTLTLSKADYEKHKVYAC |
| | (underlined) | | EVTHQGLSSPVTKSFNRGECSGGGGSGGGGSAPLKIQAYFNET |
| | and CD86 | | ADLPCQFANSQNLSLSELVVFWQDQENLVLNEVYLGKERFDS |
| | mutant 1040 | | VDSKYMGRTSFDSDSWTLRLHNLQIKDKGRYQCIIHHKKPTG |
| | | | MINIHQMNSELSVLA |
| | | | LIGHT CHAIN PREFERABLY ASSEMBLES WITH |
| | | | A HEAVY CHAIN COMPRISING THE 2404 VH |
| | | | SEQUENCE |
| | | | THUS, COMPLETE MOLECULE MAY BE |
| | | | DESIGNATED 2404/2405 |

TABLE D(1)
CDR sequences numbered according to IMGT

| VH | | | |
|---|---|---|---|
| num- | | | |
| ber | CDRH1 | CDRH2 | CDRH3 |
| 2348 | GFTFGYYY | ISSPSSYT | ARYYGSYFDY |
| | (SEQ ID NO: 76) | (SEQ ID NO: 77) | (SEQ ID NO: 78) |
| 2372 | GFTFSGYS | ISGYSMGT | ARYPWGYYFDY |
| | (SEQ ID NO: 79) | (SEQ ID NO: 80) | (SEQ ID NO: 81) |
| 2396 | GFTFSSYA | ISGSGGST | ARAYPVHGYWVFDY |
| | (SEQ ID NO: 82) | (SEQ ID NO: 83) | (SEQ ID NO: 84) |
| 2404 | GFTFSYSS | ISYSSMST | ARYSYYFDY |
| | (SEQ ID NO: 85) | (SEQ ID NO: 86) | (SEQ ID NO: 87) |

TABLE D(2)
CDR sequences numbered according to IMGT

| VL | | | |
|---|---|---|---|
| num- | | | |
| ber | CDRL1 | CDRL2 | CDRL3 |
| 2349 | QAISAY | AAS | QQSYGYYLYT |
| | (SEQ ID NO: 88) | (SEQ ID NO: 89) | (SEQ ID NO: 90) |
| 2373 | QGIRAY | AVS | QQYYYPPLST |
| | (SEQ ID NO: 91) | (SEQ ID NO: 92) | (SEQ ID NO: 93) |
| 2397 | QSISSY | AAS | QQSVSTPPT |
| | (SEQ ID NO: 94) | (SEQ ID NO: 89) | (SEQ ID NO: 95) |
| 2405 | QSISSY | AAS | QQSHYWYPLT |
| | (SEQ ID NO: 94) | (SEQ ID NO: 89) | (SEQ ID NO: 96) |

Other sequences

SEQ ID NO: 1 (human CTLA-4)
MHVAQPAVVLASSRGIASFVCEYASPGKATEVRVTVLRQADSQVTEVCAATYMMGNELTFLDDSI
CTGTSSGNQVNLTIQGLRAMDTGLYICKVELMYPPPYYLGIGNGTQIYVIAKEKKPSYNRGLCEN
APNRARM
SEQ ID NO: 2 (human CD28)
MLRLLLALNLFPSIQVTGNKILVKQSPMLVAYDNAVNLSCKYSYNLFSREFRASLHKGLDSAVEV
CVVYGNYSQQLQVYSKTGFNCDGKLGNESVTFYLQNLYVNQTDIYFCKIEVMYPPPYLDNEKSNG
TIIHVKGKHLCPSPLFPGPSKPFWVLVVVGGVLACYSLLVTVAFIIFWVRSKRSRLLHSDYMNMT
PRRPGPTRKHYQPYAPPRDFAAYRS
SEQ ID NO: 3
APLKIQAYFNETADLPCQFANSQNQSLSELVVFWQDQENLVLNEVYLGKEKFDSVHSKYMGRTSF
DSDSWTLRLHNLQIKDKGLYQCIIHHKKPTGMIRIHQMNSELSVLA
SEQ ID NO: 4
MDPQCTMGLSNILFVMAFLLSGAAPLKIQAYFNETADLPCQFANSQNQSLSELVVFWQDQENLVL
NEVYLGKEKFDSVHSKYMGRTSFDSDSWTLRLHNLQIKDKGLYQCIIHHKKPTGMIRIHQMNSEL
SVLANFSQPEIVPISNITENVYINLTCSSIHGYPEPKKMSVLLRTKNSTIEYDGIMQKSQDNVTE
LYDVSISLSVSFPDVTSNMTIFCILETDKTRLLSSPFSIELEDPQPPPDHIP
SEQ ID NO: 5
APLKIQAYFNETADLPCQFANSQNQSLSELVVFWQDQENLVLNEVYLGKEKFDSVASKYMGRTSF
DSDSWTLRLHNLQIKDKGLYQCIIHHKKPTGMIRIHQMNSELSVLA
SEQ ID NO: 44 (human CD86)
MDPQCTMGLSNILFVMAFLLSGAAPLKIQAYFNETADLPCQFANSQNQSLSELVVFWQDQENLVL
NEVYLGKEKFDSVHSKYMGRTSFDSDSWTLRLHNLQIKDKGLYQCIIHHKKPTGMIRIHQMNSEL
SVLANFSQPEIVPISNITENVYINLTCSSIHGYPEPKKMSVLLRTKNSTIEYDGIMQKSQDNVTE
LYDVSISLSVSFPDVTSNMTIFCILETDKTRLLSSPFSIELEDPQPPPDHIPWITAVLPTVIICV
MVFCLILWKWKKKKRPRNSYKCGTNTMEREESEQTKKREKIHIPERSDEAQRVFKSSKTSSCDKS
DTCF
SEQ ID NO: 45 (murine CTLA-4)
MACLGLRRYKAQLQLPSRTWPFVALLTLLFIPVFSEAIQVTQPSVVLASSHGVASFPCEYSPSHN
TDEVRVTVLRQTNDQMTEVCATTFTEKNTVGFLDYPFCSGTFNESRVNLTIQGLRAVDTGLYLCK
VELMYPPPYFVGMGNGTQIYVIDPEPCPDSDFLLWILVAVSLGLFFYSFLVSAVSLSKMLKKRSP
LTTGVYVKMPPTEPECEKQFQPYFIPIN
SEQ ID NO: 46 (murine CD28)
MTLRLLFLALNFFSVQVTENKILVKQSPLLVVDSNEVSLSCRYSYNLLAKEFRASLYKGVNSDVE
VCVGNGNFTYQPQFRSNAEFNCDGDFDNETVTFRLWNLHVNHTDIYFCKIEFMYPPPYLDNERSN
GTIIHIKEKHLCHTQSSPKLFWALVVVAGVLFCYGLLVTVALCVIWTNSRRNRLLQVTTMNMTPR
RPGLTRKPYQPYAPARDFAAYRP
SEQ ID NO: 47 (linker sequence)
SGGGGSGGGGS
SEQ ID NO: 48 (linker sequence)
SGGGGSGGGGSAP
SEQ ID NO: 49 (linker sequence)
NFSQP
SEQ ID NO: 50 (linker sequence)
KRTVA
SEQ ID NO: 51 (linker sequence)
GGGGSGGGGSGGGGS
SEQ ID NO: 97 (IgG1 heavy chain constant region)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQKSLS
LSPGK
SEQ ID NO: 98 (kappa chain constant region)
RTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
SEQ ID NO: 99 (modified IgG4 constant region)
ASTKGPSVFPLAPCSRSTSESTAALGCLVKDYFPEPVTVSWNSGALTSGVHTFPAVLQSSGLYSL
SSVVTVPSSSLGTKTYTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNRYTQKSLSLSL
GK
SEQ ID NO: 100 (IgG4 constant region)
ASTKGPSVFPLAPCSRSTSESTAALGCLVKDYFPEPVTVSWNSGALTSGVHTFPAVLQSSGLYSL
SSVVTVPSSSLGTKTYTCNVDHKPSNTKVDKRVESKYGPPCPSCPAPEFLGGPSVFLFPPKPKDT
LMISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSL
GK
SEQ ID NO: 101 (modified IgG4 constant region)
ASTKGPSVFPLAPCSRSTSESTAALGCLVKDYFPEPVTVSWNSGALTSGVHTFPAVLQSSGLYSL
SSVVTVPSSSLGTKTYTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSL
GK
SEQ ID NO: 102
gcttccacca agggcccatc cgtcttcccc ctggcgccct gctccaggag cacctccgag
agcacagccg ccctgggctg cctggtcaag gactacttcc ccgaaccggt gacggtgtcg
tggaactcag gcgccctgac cagcggcgtg cacaccttcc cggctgtcct acagtcctca
ggactctact ccctcagcag cgtggtgacc gtgccctcca gcagcttggg cacgaagacc
tacacctgca acgtagatca caagcccagc aacaccaagg tggacaagag agttgagtcc
aaatatggtc ccccatgccc accttgccca gcacctgagt tcctgggggg accatcagtc
ttcctgttcc ccccaaaacc caaggacact ctcatgatct cccggacccc tgaggtcacg
tgcgtggtgg tggacgtgag ccaggaagac cccgaggtcc agttcaactg gtacgtggat
ggcgtggagg tgcataatgc caagacaaag ccgcgggagg agcagttcaa cagcacgtac
cgtgtggtca gcgtcctcac cgtcctgcac caggactggc tgaacggcaa ggagtacaag
tgcaaggtct ccaacaaagg cctcccgtcc tccatcgaga aaaccatctc caaagccaaa
gggcagcccc gagagccaca ggtgtacacc ctgcccccat cccaggagga gatgaccaag
aaccaggtca gcctgacctg cctggtcaaa ggcttctacc ccagcgacat cgccgtggag
tgggagagca atgggcagcc ggagaacaac tacaagacca cgcctcccgt gctggactcc
gacggctcct tcttcctcta cagcaggcta accgtggaca agagcaggtg gcaggagggg
aatgtcttct catgctccgt gatgcatgag gctctgcaca accgctacac acagaagagc
ctctccctgt ctctgggtaa a
SEQ ID NO: 103
agctttctgg ggcaggccgg gcctgacttt ggctgggggc agggaggggg ctaaggtgac
gcaggtggcg ccagccaggt gcacacccaa tgcccatgag cccagacact ggaccctgca
tggaccatcg cggatagaca agaaccgagg ggcctctgcg ccctgggccc agctctgtcc
cacaccgcgg tcacatggca ccacctctct tgcagcttcc accaagggcc catccgtctt
ccccctggcg ccctgctcca ggagcacctc cgagagcaca gccgccctgg gctgcctggt
caaggactac ttccccgaac cggtgacggt gtcgtggaac tcaggcgccc tgaccagcgg
cgtgcacacc ttcccggctg tcctacagtc ctcaggactc tactccctca gcagcgtggt
gaccgtgccc tccagcagct tgggcacgaa gacctacacc tgcaacgtag atcacaagcc
cagcaacacc aaggtggaca agagagttgg tgagaggcca gcacagggag ggagggtgtc
tgctggaagc caggctcagc cctcctgcct ggacgcaccc cggctgtgca gccccagccc
agggcagcaa ggcatgcccc atctgtctcc tcacccggag gcctctgacc accccactca
tgctcaggga gagggtcttc tggatttttc caccaggctc ccggcaccac aggctggatg
cccctacccc aggccctgcg catacagggc aggtgctgcg ctcagacctg ccaagagcca
tatccgggag gaccctgccc ctgacctaag cccaccccaa aggccaaact ctccactccc
tcagctcaga caccttctct cctcccagat ctgagtaact cccaatcttc tctctgcaga
gtccaaatat ggtcccccat gcccaccttg cccaggtaag ccaacccagg cctcgccctc
cagctcaagg cgggacaggt gccctagagt agcctgcatc cagggacagg ccccagccgg
gtgctgacgc atccacctcc atctcttcct cagcacctga gttcctgggg ggaccatcag
tcttcctgtt ccccccaaaa cccaaggaca ctctcatgat ctcccggacc cctgaggtca
cgtgcgtggt ggtggacgtg agccaggaag accccgaggt ccagttcaac tggtacgtgg
atggcgtgga ggtgcataat gccaagacaa agccgcggga ggagcagttc aacagcacgt
accgtgtggt cagcgtcctc accgtcctgc accaggactg gctgaacggc aaggagtaca
agtgcaaggt ctccaacaaa ggcctcccgt cctccatcga gaaaaccatc tccaaagcca
aaggtgggac ccacggggtg cgagggccac acggacagag gccagctcgg cccaccctct
gccctgggag tgaccgctgt gccaacctct gtccctacag ggcagccccg agagccacag
gtgtacaccc tgcccccatc ccaggaggag atgaccaaga accaggtcag cctgacctgc
ctggtcaaag gcttctaccc cagcgacatc gccgtggagt gggagagcaa tgggcagccg
gagaacaact acaagaccac gcctcccgtg ctggactccg acggctcctt cttcctctac
agcaggctaa ccgtggacaa gagcaggtgg caggagggga atgtcttctc atgctccgtg
atgcatgagg ctctgcacaa ccgctacaca cagaagagcc tctccctgtc tctgggtaaa
tgagtgccag ggccggcaag cccccgctcc ccgggctctc ggggtcgcgc gaggatgctt
ggcacgtacc ccgtctacat acttcccagg cacccagcat ggaaataaag cacccaccac
tgccctgggc ccctgtgaga ctgtgatggt tctttccacg ggtcaggccg agtctgaggc
ctgagtgaca tgagggaggc agagcgggtc ccactgtccc cacactgg
SEQ ID NO: 104
gcttccacca agggcccatc cgtcttcccc ctggcgccct gctccaggag cacctccgag
agcacagccg ccctgggctg cctggtcaag gactacttcc ccgaaccggt gacggtgtcg
tggaactcag gcgccctgac cagcggcgtg cacaccttcc cggctgtcct acagtcctca
ggactctact ccctcagcag cgtggtgacc gtgccctcca gcagcttggg cacgaagacc
tacacctgca acgtagatca caagcccagc aacaccaagg tggacaagag agttgagtcc
aaatatggtc ccccatgccc atcatgccca gcacctgagt tcctgggggg accatcagtc
ttcctgttcc ccccaaaacc caaggacact ctcatgatct cccggacccc tgaggtcacg
tgcgtggtgg tggacgtgag ccaggaagac cccgaggtcc agttcaactg gtacgtggat
ggcgtggagg tgcataatgc caagacaaag ccgcgggagg agcagttcaa cagcacgtac
cgtgtggtca gcgtcctcac cgtcctgcac caggactggc tgaacggcaa ggagtacaag
tgcaaggtct ccaacaaagg cctcccgtcc tccatcgaga aaaccatctc caaagccaaa
gggcagcccc gagagccaca ggtgtacacc ctgcccccat cccaggagga gatgaccaag
aaccaggtca gcctgacctg cctggtcaaa ggcttctacc ccagcgacat cgccgtggag
tgggagagca atgggcagcc ggagaacaac tacaagacca cgcctcccgt gctggactcc
gacggctcct tcttcctcta cagcaggcta accgtggaca agagcaggtg gcaggagggg
aatgtcttct catgctccgt gatgcatgag gctctgcaca accactacac acagaagagc
ctctccctgt ctctgggtaa a
SEQ ID NO: 105
agctttctgg ggcaggccgg gcctgacttt ggctgggggc agggaggggg ctaaggtgac
gcaggtggcg ccagccaggt gcacacccaa tgcccatgag cccagacact ggaccctgca
tggaccatcg cggatagaca agaaccgagg ggcctctgcg ccctgggccc agctctgtcc
cacaccgcgg tcacatggca ccacctctct tgcagcttcc accaagggcc catccgtctt
ccccctggcg ccctgctcca ggagcacctc cgagagcaca gccgccctgg gctgcctggt
caaggactac ttccccgaac cggtgacggt gtcgtggaac tcaggcgccc tgaccagcgg
cgtgcacacc ttcccggctg tcctacagtc ctcaggactc tactccctca gcagcgtggt
gaccgtgccc tccagcagct tgggcacgaa gacctacacc tgcaacgtag atcacaagcc
cagcaacacc aaggtggaca agagagttgg tgagaggcca gcacagggag ggagggtgtc
tgctggaagc caggctcagc cctcctgcct ggacgcaccc cggctgtgca gccccagccc
agggcagcaa ggcatgcccc atctgtctcc tcacccggag gcctctgacc accccactca
tgctcaggga gagggtcttc tggatttttc caccaggctc ccggcaccac aggctggatg
cccctacccc aggccctgcg catacagggc aggtgctgcg ctcagacctg ccaagagcca
tatccgggag gaccctgccc ctgacctaag cccaccccaa aggccaaact ctccactccc
tcagctcaga caccttctct cctcccagat ctgagtaact cccaatcttc tctctgcaga
gtccaaatat ggtcccccat gcccatcatg cccaggtaag ccaacccagg cctcgccctc
cagctcaagg cgggacaggt gccctagagt agcctgcatc cagggacagg ccccagccgg
gtgctgacgc atccacctcc atctcttcct cagcacctga gttcctgggg ggaccatcag
tcttcctgtt ccccccaaaa cccaaggaca ctctcatgat ctcccggacc cctgaggtca
cgtgcgtggt ggtggacgtg agccaggaag accccgaggt ccagttcaac tggtacgtgg
atggcgtgga ggtgcataat gccaagacaa agccgcggga ggagcagttc aacagcacgt
accgtgtggt cagcgtcctc accgtcctgc accaggactg gctgaacggc aaggagtaca
agtgcaaggt ctccaacaaa ggcctcccgt cctccatcga gaaaaccatc tccaaagcca
aaggtgggac ccacggggtg cgagggccac acggacagag gccagctcgg cccaccctct
gccctgggag tgaccgctgt gccaacctct gtccctacag ggcagccccg agagccacag
gtgtacaccc tgcccccatc ccaggaggag atgaccaaga accaggtcag cctgacctgc
ctggtcaaag gcttctaccc cagcgacatc gccgtggagt gggagagcaa tgggcagccg
gagaacaact acaagaccac gcctcccgtg ctggactccg acggctcctt cttcctctac
agcaggctaa ccgtggacaa gagcaggtgg caggagggga atgtcttctc atgctccgtg
atgcatgagg ctctgcacaa ccactacaca cagaagagcc tctccctgtc tctgggtaaa
tgagtgccag ggccggcaag cccccgctcc ccgggctctc ggggtcgcgc gaggatgctt
ggcacgtacc ccgtctacat acttcccagg cacccagcat ggaaataaag cacccaccac
tgccctgggc ccctgtgaga ctgtgatggt tctttccacg ggtcaggccg agtctgaggc
ctgagtgaca tgagggaggc agagcgggtc ccactgtccc cacactgg
SEQ ID NO: 106
gcctccacca agggcccatc ggtcttcccc ctggcaccct cctccaagag cacctctggg
ggcacagcgg ccctgggctg cctggtcaag gactacttcc ccgaaccggt gacggtgtcg
tggaactcag gcgccctgac cagcggcgtg cacaccttcc cggctgtcct acagtcctca
ggactctact ccctcagcag cgtggtgacc gtgccctcca gcagcttggg cacccagacc
tacatctgca acgtgaatca caagcccagc aacaccaagg tggacaagaa agttgagccc
aaatcttgtg acaaaactca cacatgccca ccgtgcccag cacctgaact cctgggggga
ccgtcagtct tcctcttccc cccaaaaccc aaggacaccc tcatgatctc ccggacccct
gaggtcacat gcgtggtggt ggacgtgagc cacgaagacc ctgaggtcaa gttcaactgg
tacgtggacg gcgtggaggt gcataatgcc aagacaaagc cgcgggagga gcagtacaac
agcacgtacc gtgtggtcag cgtcctcacc gtcctgcacc aggactggct gaatggcaag
gagtacaagt gcaaggtctc caacaaagcc ctcccagccc ccatcgagaa aaccatctcc
aaagccaaag ggcagccccg agaaccacag gtgtacaccc tgcccccatc ccgggatgag
ctgaccaaga accaggtcag cctgacctgc ctggtcaaag gcttctatcc cagcgacatc
gccgtggagt gggagagcaa tgggcagccg gagaacaact acaagaccac gcctcccgtg
ctggactccg acggctcctt cttcctctac agcaagctca ccgtggacaa gagcaggtgg
cagcagggga acgtcttctc atgctccgtg atgcatgagg ctctgcacaa ccactacacg
cagaagagcc tctccctgtc tccgggtaaa
SEQ ID NO: 107
gcctccacca agggcccatc ggtcttcccc ctggcaccct cctccaagag cacctctggg
ggcacagcgg ccctgggctg cctggtcaag gactacttcc ccgaaccggt gacggtgtcg
tggaactcag gcgccctgac cagcggcgtg cacaccttcc cggctgtcct acagtcctca
ggactctact ccctcagcag cgtggtgacc gtgccctcca gcagcttggg cacccagacc
tacatctgca acgtgaatca caagcccagc aacaccaagg tggacaagaa agttggtgag
aggccagcac agggagggag ggtgtctgct ggaagccagg ctcagcgctc ctgcctggac
gcatcccggc tatgcagccc cagtccaggg cagcaaggca ggccccgtct gcctcttcac
ccggaggcct ctgcccgccc cactcatgct cagggagagg gtcttctggc tttttcccca
ggctctgggc aggcacaggc taggtgcccc taacccaggc cctgcacaca aaggggcagg
tgctgggctc agacctgcca agagccatat ccgggaggac cctgcccctg acctaagccc
accccaaagg ccaaactctc cactccctca gctcggacac cttctctcct cccagattcc
agtaactccc aatcttctct ctgcagagcc caaatcttgt gacaaaactc acacatgccc
accgtgccca ggtaagccag cccaggcctc gccctccagc tcaaggcggg acaggtgccc
tagagtagcc tgcatccagg gacaggcccc agccgggtgc tgacacgtcc acctccatct
cttcctcagc acctgaactc ctggggggac cgtcagtctt cctcttcccc ccaaaaccca
aggacaccct catgatctcc cggacccctg aggtcacatg cgtggtggtg gacgtgagcc
acgaagaccc tgaggtcaag ttcaactggt acgtggacgg cgtggaggtg cataatgcca
agacaaagcc gcgggaggag cagtacaaca gcacgtaccg tgtggtcagc gtcctcaccg
tcctgcacca ggactggctg aatggcaagg agtacaagtg caaggtctcc aacaaagccc
tcccagcccc catcgagaaa accatctcca aagccaaagg tgggacccgt ggggtgcgag
ggccacatgg acagaggccg gctcggccca ccctctgccc tgagagtgac cgctgtacca
acctctgtcc ctacagggca gccccgagaa ccacaggtgt acaccctgcc cccatcccgg
gatgagctga ccaagaacca ggtcagcctg acctgcctgg tcaaaggctt ctatcccagc
gacatcgccg tggagtggga gagcaatggg cagccggaga acaactacaa gaccacgcct
cccgtgctgg actccgacgg ctccttcttc ctctacagca agctcaccgt ggacaagagc
aggtggcagc aggggaacgt cttctcatgc tccgtgatgc atgaggctct gcacaaccac
tacacgcaga agagcctctc cctgtctccg ggtaaa
SEQ ID NO: 108
cgaactgtgg ctgcaccatc tgtcttcatc ttcccgccat ctgatgagca gttgaaatct
ggaactgcct ctgttgtgtg cctgctgaat aacttctatc ccagagaggc caaagtacag
tggaaggtgg ataacgccct ccaatcgggt aactcccagg agagtgtcac agagcaggac
agcaaggaca gcacctacag cctcagcagc accctgacgc tgagcaaagc agactacgag
aaacacaaag tctacgcctg cgaagtcacc catcagggcc tgagctcgcc cgtcacaaag
agcttcaaca ggggagagtg t
SEQ ID NO: 109
gcttccacca agggcccatc cgtcttcccc ctggcgccct gctccaggag cacctccgag
agcacagccg ccctgggctg cctggtcaag gactacttcc ccgaaccggt gacggtgtcg
tggaactcag gcgccctgac cagcggcgtg cacaccttcc cggctgtcct acagtcctca
ggactctact ccctcagcag cgtggtgacc gtgccctcca gcagcttggg cacgaagacc
tacacctgca acgtagatca caagcccagc aacaccaagg tggacaagag agttgagtcc
aaatatggtc ccccatgccc accttgccca gcacctgagt tcctgggggg accatcagtc
ttcctgttcc ccccaaaacc caaggacact ctcatgatct cccggacccc tgaggtcacg
tgcgtggtgg tggacgtgag ccaggaagac cccgaggtcc agttcaactg gtacgtggat
ggcgtggagg tgcataatgc caagacaaag ccgcgggagg agcagttcaa cagcacgtac
cgtgtggtca gcgtcctcac cgtcctgcac caggactggc tgaacggcaa ggagtacaag
tgcaaggtct ccaacaaagg cctcccgtcc tccatcgaga aaaccatctc caaagccaaa
gggcagcccc gagagccaca ggtgtacacc ctgcccccat cccaggagga gatgaccaag
aaccaggtca gcctgacctg cctggtcaaa ggcttctacc ccagcgacat cgccgtggag
tgggagagca atgggcagcc ggagaacaac tacaagacca cgcctcccgt gctggactcc
gacggctcct tcttcctcta cagcaggcta accgtggaca agagcaggtg gcaggagggg
aatgtcttct catgctccgt gatgcatgag gctctgcaca accactacac acagaagagc
ctctccctgt ctctgggtaa a
SEQ ID NO: 110
agctttctgg ggcaggccgg gcctgacttt ggctgggggc agggaggggg ctaaggtgac
gcaggtggcg ccagccaggt gcacacccaa tgcccatgag cccagacact ggaccctgca
tggaccatcg cggatagaca agaaccgagg ggcctctgcg ccctgggccc agctctgtcc
cacaccgcgg tcacatggca ccacctctct tgcagcttcc accaagggcc catccgtctt
ccccctggcg ccctgctcca ggagcacctc cgagagcaca gccgccctgg gctgcctggt
caaggactac ttccccgaac cggtgacggt gtcgtggaac tcaggcgccc tgaccagcgg
cgtgcacacc ttcccggctg tcctacagtc ctcaggactc tactccctca gcagcgtggt
gaccgtgccc tccagcagct tgggcacgaa gacctacacc tgcaacgtag atcacaagcc
cagcaacacc aaggtggaca agagagttgg tgagaggcca gcacagggag ggagggtgtc
tgctggaagc caggctcagc cctcctgcct ggacgcaccc cggctgtgca gccccagccc
agggcagcaa ggcatgcccc atctgtctcc tcacccggag gcctctgacc accccactca
tgctcaggga gagggtcttc tggatttttc caccaggctc ccggcaccac aggctggatg
cccctacccc aggccctgcg catacagggc aggtgctgcg ctcagacctg ccaagagcca
tatccgggag gaccctgccc ctgacctaag cccaccccaa aggccaaact ctccactccc
tcagctcaga caccttctct cctcccagat ctgagtaact cccaatcttc tctctgcaga
gtccaaatat ggtcccccat gcccaccttg cccaggtaag ccaacccagg cctcgccctc
cagctcaagg cgggacaggt gccctagagt agcctgcatc cagggacagg ccccagccgg
gtgctgacgc atccacctcc atctcttcct cagcacctga gttcctgggg ggaccatcag
tcttcctgtt ccccccaaaa cccaaggaca ctctcatgat ctcccggacc cctgaggtca
cgtgcgtggt ggtggacgtg agccaggaag accccgaggt ccagttcaac tggtacgtgg
atggcgtgga ggtgcataat gccaagacaa agccgcggga ggagcagttc aacagcacgt
accgtgtggt cagcgtcctc accgtcctgc accaggactg gctgaacggc aaggagtaca
agtgcaaggt ctccaacaaa ggcctcccgt cctccatcga gaaaaccatc tccaaagcca
aaggtgggac ccacggggtg cgagggccac acggacagag gccagctcgg cccaccctct
gccctgggag tgaccgctgt gccaacctct gtccctacag ggcagccccg agagccacag
gtgtacaccc tgcccccatc ccaggaggag atgaccaaga accaggtcag cctgacctgc
ctggtcaaag gcttctaccc cagcgacatc gccgtggagt gggagagcaa tgggcagccg
gagaacaact acaagaccac gcctcccgtg ctggactccg acggctcctt cttcctctac
agcaggctaa ccgtggacaa gagcaggtgg caggagggga atgtcttctc atgctccgtg
atgcatgagg ctctgcacaa ccactacaca cagaagagcc tctccctgtc tctgggtaaa
tgagtgccag ggccggcaag cccccgctcc ccgggctctc ggggtcgcgc gaggatgctt
ggcacgtacc ccgtctacat acttcccagg cacccagcat ggaaataaag cacccaccac
tgccctgggc ccctgtgaga ctgtgatggt tctttccacg ggtcaggccg agtctgaggc
ctgagtgaca tgagggaggc agagcgggtc ccactgtccc cacactgg
SEQ ID NO: 111
MAQHGAMGAF RALCGLALLC ALSLGQRPTG GPGCGPGRLL LGTGTDARCC RVHTTRCCRD
YPGEECCSEW DCMCVQPEFH CGDPCCTTCR HHPCPPGQGV QSQGKFSFGF QCIDCASGTF
SGGHEGHCKP WTDCTQFGFL TVFPGNKTHN AVCVPGSPPA EPLGWLTVVL LAVAACVLLL
TSAQLGLHIW QLRSQCMWPR ETQLLLEVPP STEDARSCQF PEEERGERSA EEKGRLGDLW
V

### EXAMPLES

The present invention is further illustrated by the following examples which should not be construed as further limiting. The contents of all figures and all references, patents and published patent applications cited throughout this application are expressly incorporated herein by reference.

### Example 1—Dual ELISA of Bispecific Antibodies GITR/CTLA-4

### Material and Methods

ELISA plates were coated with GITR-hFc (0.5 ug/ml) 50 ul/well (R&D Systems, #689-GR). The plates were then washed 3 times with PBST (PBS+0.05% polysorbate 20) and blocked with PBST and 1% BSA for 1 h at room temperature. After 3 washes with PBST, the bispecific antibodies were added at different concentrations (highest concentration 66.7 nM) and incubated for 1 h at room temperature. The plates were washed as above and 0.1 μg/ml biotinylated CTLA-4-mFc (Ancell, #501-030) was added and incubated for 1 h at room temperature. After three washes with PBST, HRP-labeled streptavidin was added and incubated for 1 h at room temperature. The plates were washed 6 times with PBST and SuperSignal Pico Luminescent substrate (Thermo Scientific, #37069) was added according to the manufacturer's protocol and the luminescence was measured in a Fluorostar Optima (BMG labtech).

### Results and Conclusions

The bispecific antibodies can bind to both targets simultaneously (FIG. 1) in a dose-dependent manner, which is important for the proposed mode of action. No difference in target binding is seen with the afucosylated bispecific antibody format (FIG. 2). The EC50 values are 0.64 and 0.54 nM for the wildtype and afucosylated antibodies, respectively.

### Example 2—Kinetics of Bispecific Antibody Interactions with GITR

### Material and Methods

Kinetic measurements were performed using the Octet RED96 platform equipped with AR2G (Amine Reactive 2nd Gen) sensor tips (ForteBio). Human GITR (Acro Biosystems, #GIR-H5228) was coupled to the biosensor surface in 10 mM sodium acetate (pH 5.0) using standard amine coupling with 20 mM 1-ethyl-3-(3-dimethylaminopropyl)carbodiimide hydrochloride (EDC), 10 mM N-hydroxysuccinimide (NHS), and 1 M ethanolamine-HCl (pH 8.5). Bispecific antibodies were diluted in 1× Kinetics Buffer (ForteBio) to 80 nM, 40 nM, 20 nM, 10 nM, 5 nM, 2.5 nM and 1.25 nM. Binding kinetics was studied in 1× Kinetics buffer where association was allowed for 300 sec followed by dissociation for 900 sec. Sensor tips were regenerated using 10 mM glycine, pH 1.7. Data generated was referenced by subtracting a parallel buffer blank, the baseline was aligned with the y-axis, inter-step correlation by alignment against dissociation was performed and the data was smoothed by a Savitzky-Golay filter in the data analysis software (v. 9.0.0.14). The processed data was fitted using a 1:1 Langmuir binding model with X² as a measurement of fitting accuracy.

### Results and Conclusions

As summarized in Table 1 below, and FIG. 3, the bispecific antibodies bind to GITR with KD in the low nM to sub-nM range using the above described assay setup. X² values confirms good curve fitting.

TABLE 1
Summary of kinetic profiles of bispecific antibody interactions with GITR.

| Bispecific | | | | |
|---|---|---|---|---|
| antibody | kₐ (M⁻¹ s⁻¹) | k_{d} (s⁻¹) | K_{D} (M) | X² (nm) |
| 2348/2349 | 1.53 × 10⁵ | 5.28 × 10⁻⁵ | 3.46 × 10⁻¹⁰ | 0.0245 |
| 2372/2373 | 3.51 × 10⁵ | 2.14 × 10⁻⁴ | 6.08 × 10⁻¹⁰ | 0.4943 |
| 2396/2397 | 2.17 × 10⁵ | 1.46 × 10⁻⁴ | 6.73 × 10⁻¹⁰ | 0.0891 |
| 2404/2405 | 2.54 × 10⁵ | 4.23 × 10⁻⁴ | 1.67 × 10⁻⁹ | 0.2305 |

### Example 3—Kinetics of the Interaction of Bispecific Antibodies with CTLA-4

### Material and Methods

Kinetic measurements were performed using the Octet RED96 platform equipped with Anti-hIgG Fc Capture (AHC) sensor tips (ForteBio). Bispecific antibodies were diluted to 2 μg/ml in 1× Kinetics Buffer (ForteBio) and loaded to sensors tips for 300 seconds. The immobilized bispecific antibodies were then assayed against 4 2-fold dilutions of human CTLA-4 (ACRO Biosystems, #CT4-H5229). Binding kinetics was studied in 1× Kinetics buffer where association was allowed for 180 sec followed by dissociation for 600 sec. Sensor tips were regenerated using 10 mM glycine, pH 1.7. Data generated was referenced by subtracting a parallel buffer blank, the baseline was aligned with the y-axis, inter-step correlation by alignment against dissociation was performed and the data was smoothed by a Savitzky-Golay filter in the data analysis software (v. 9.0.0.14). The processed data was fitted using a 1:1 Langmuir binding model with X² as a measurement of fitting accuracy.

### Results and Conclusions

As summarized in Table 2 below, and FIG. 4, the CTLA-4 binder 2372/2373 interacts with CTLA-4 with KD in the nM range using the above described assay setup. X² values confirms good curve fitting.

TABLE 2
Summary of the kinetic profile of 2372/2373 interaction with CTLA-4.

| kₐ (M⁻¹ s⁻¹) | k_{d} (s⁻¹) | K_{D} (M) | X² (nm) |
|---|---|---|---|
| 1.90 × 10⁵ | 5.51 × 10⁻⁴ | 2.9 × 10⁻⁹ | 0.0754 |

### Example 4—Ability of GITR/CTLA-4 Bispecific Antibodies to Block Interaction Between GITR and GITR Ligand

### Material and Methods

Ligand blocking experiments were performed using the Octet RED96 platform equipped with AR2G (Amine Reactive 2nd Gen) sensor tips (ForteBio). Human GITR (Acro Biosystems, # GIR-H5228) was coupled to the biosensor surface in 10 mM sodium acetate (pH 5.0) using standard amine coupling with 20 mM 1-ethyl-3-(3-dimethyl-aminopropyl)-carbodiimide hydrochloride (EDC), 10 mM N-hydroxysuccinimide (NHS), and 1 M ethanolamine-HCl (pH 8.5). Bispecific antibodies were diluted to 80 nM and GITR Ligand (Acro Biosystems, # GIL-H526a) to 5 μg/ml in 1× Kinetics Buffer (ForteBio). Each bispecific antibody was allowed to bind to two parallel biosensor tips for 600 sec prior to dipping one sensor in GITR Ligand solution (assay sensor) and one sensor in 1× Kinetics buffer (reference sensor) for 300 sec. One pair of biosensors were run in 1× Kinetics buffer without any bispecific antibody to demonstrate GITR Ligand binding without inhibition. Finally, dissociation of formed GITR-GITR Ligand complexes in 1× Kinetics Buffer were followed for 120 sec prior to sensor tip regeneration using 10 mM glycine, pH 1.7.

### Results and Conclusions

As shown in FIG. 5, the bispecific antibodies bind to GITR in a way that completely or partially blocks the ability of GITR to interact with GITR Ligand. 2372/2373 and 2404/2405 almost completely block the GITR ligand.

### Example 5—Ability of GITR/CTLA-4 Bispecific Antibodies to Block Each Other's Interaction with GITR

### Material and Methods

Blocking experiments were performed using the Octet RED96 platform equipped with AR2G (Amine Reactive 2nd Gen) sensor tips (ForteBio). Human GITR (Acro Biosystems, # GIR-H5228) was coupled to the biosensor surface in 10 mM sodium acetate (pH 5.0) using standard amine coupling with 20 mM 1-ethyl-3-(3-dimethylaminopropyl)carbodiimide hydrochloride (EDC), 10 mM N-hydroxysuccinimide (NHS), and 1 M ethanolamine-HCl (pH 8.5). Bispecific antibodies were diluted to either 80 nM (primary bispecific antibodies) or 20 nM (secondary bispecific antibodies and control mAb) in 1× Kinetics Buffer (ForteBio). As control a commercially available GITR specific monospecific mAb (DT5D3, Miltenyi Biotec) was used. Two biosensor tips were used for each assay. Primary bispecific antibodies were allowed to bind to one of these sensors (assay sensor) for 600 sec while the other sensor was incubated in 1× Kinetics Buffer (reference sensor). Next, the two sensors were incubated in wells containing the secondary antibodies and binding was studied for 180 sec prior to regeneration of the sensors using 10 mM glycine, pH 1.7.

### Results and Conclusions

As exemplified in FIG. 6, the bispecific antibodies possess the ability to at least in part inhibit the binding of all analysed secondary antibodies (bispecific as well as control mAb) to GITR. The assay was repeated using all four bispecific antibodies as primary antibody, with similar results in all setups (data not shown). This indicates that all antibodies included in this assay bind to epitopes that overlap or at least in such close proximity that they block each other's binding to GITR, or interfere with binding to the receptor by steric hindrance or by inducing conformational changes in GITR.

### Example 6—Binding to Target-Expressing Cells of GITR/CTLA-4 Bispecific Antibodies

Binding of GITR/CTLA-4 bispecific antibodies to target-expressing cells was assessed by flow cytometry. The afucosylated format was compared to wildtype IgG1. No difference in the target-binding capacity was expected.

### Material and Methods

Transfected CHO cells stably expressing high levels of GITR and CTLA-4 (CHO-GITR^{hi}-CTLA-4^{hi} cells) were used. 250,000 cells/well was stained with serially diluted GITR/CTLA-4 bispecific antibodies in FACS buffer (PBS with 0.5% BSA) for 1 h at 4° C. Cells were washed in FACS buffer followed by the addition of a secondary PE-conjugated anti-hFc antibody (Jackson, #109-115-098) diluted 1:100 in FACS buffer. After a 30-min incubation at 4° C., cells were washed twice, resuspended in FACS buffer and analysed on a FACS Verse.

### Results and Conclusions

As seen in FIG. 7, no difference in target binding is seen. The EC50 values of the wildtype and afucosylated antibodies are 11.7 and 9.9 nM, respectively.

### Example 7—Fc Receptor Binding of GITR/CTLA-4 Bispecific Antibodies with Afucosylated Fc Domain

In addition to antigen binding, antibodies can engage Fc-gamma receptors (FcγRs) through interactions with the constant domains. These interactions mediate effector function such as antibody-dependent cellular cytotoxicity (ADCC), antibody-dependent cellular phagocytosis (ADCP) and complement-dependent cytotoxicity (CDC). Effector function activity is high for the IgG1 isotype, but low for IgG2 and IgG4. It is sometimes desirable to enhance the effector functions of IgG1 antibodies, particularly ADCC. This can be achieved e.g. through the introduction of mutations or through afucosylation. Here, we have compared a wildtype and afucosylated GITR/CTLA-4 bispecific antibody for its binding to human and mouse FcγRs. An enhanced binding to human FcγRIIIa is expected with the afucosylated format.

### Material and Methods

FcγR affinity was determined using the Octet RED96 platform equipped with Anti-Human Fab-CH1 (FAB2G) sensor tips (ForteBio). Bispecific antibodies were diluted to 200 nM in 1× Kinetics Buffer (ForteBio) and loaded to a set of 8 parallel sensors for 300 seconds to reach an immobilization response of >1.5 nm. The immobilized bispecific antibodies were then assayed against 7 2-fold dilutions of FcγRs, starting at 100 nM for human FcγRI and 1 μM for all other assayed FcγRs. One immobilized sensor was assayed against 1× Kinetics Buffer for referencing and the entire assay was repeated without immobilization of bispecific antibodies to allow for double referencing. FcγRs included were obtained from R&D Systems (human FcγRI, #1257-FC-050; human FcγRIIa, #1330-CD-050; human FcγRIIb, #1460-CD-050; human FcγRIIIa (V158), #4325-FC-050; human FcγRIIIa (F158), #8894-FC-050; mouse FcγRI, #2074-FC-050; mouse FcγRIIb, #1875-CD-050; mouse FcγRIII, #1960-FC-050) and Sino Biologicals (mouse FcγRIV, #50036-M27H-50). Binding to FcγRs was carried out for 60 seconds, followed by dissociation for 60 seconds in 1× Kinetics Buffer and regeneration of sensor tips using 10 mM glycine, pH 1.7. Data generated was referenced by standard double referencing, the baseline was aligned with the y-axis, inter-step correlation by alignment against dissociation was performed and the data was smoothed by a Savitzky-Golay filtering in the data analysis software (v. 9.0.0.14). The processed data was fitted using a 1:1 Langmuir binding model with X² as a measurement of fitting accuracy. To improve curve fitting quality of dissociation curves generated against FcγRs with very fast dissociation rates, only the initial 10 seconds of the dissociation curves were included in the curve fitting.

### Results and Conclusions

The obtained affinity constants (K_{D}) of assessed bispecific antibodies against the set of FcγRs are summarized in Table 3 and Table 4. As expected, afucosylation of 2372/2373 led to an increased affinity for human FcγRIIIa (both V158 and F158 variants). In addition to this, the afucosylated versions of 2372/2373 and the bispecific surrogate antibody bound mouse FcγRIV with a 2.1-2.5-fold increased affinity compared to the wild-type versions of these bispecific antibodies.

TABLE 3
Summary of affinity constants (K_{D}, nM) of wildtype and afucosylated GITR/CTLA-4 bispecific
antibodies to human Fcγ receptors.

| | FcγRI¹ | | FcγRIIa | | FcyRIIb² | | FcγRIIIa (V158) | | FcγRIIIa (F158) | |
|---|---|---|---|---|---|---|---|---|---|---|
| | K_{D} | Fold³ | K_{D} | Fold | K_{D} | Fold | K_{D} | Fold | K_{D} | Fold |
| 2372/2373 WT | 0.04 | N/A | 845 | 1.3 | 270 | 0.7 | 471 | 15.7 | 1070 | 9.3 |
| 2372/2373 AF | <0.01 | | 635 | | 418 | | 30 | | 115 | |

¹The very slow dissociation rate of formed complexes reduces accuracy of determined dissociation rate constants and consequently also the affinity constants
²Low responses due to the low affinity of these interactions significantly reduces curve fitting quality
³Fold = K_{D} 2372/2373 WT/K_{D} 2372/2373 AF

TABLE 4
Summary of affinity constants (K_{D}, nM) of wildtype
and afucosylated 2372/2373 and surrogate GITR/CTLA-4
bispecific antibody to mouse Fcγ receptors

| | FcγRI | | FcγRIIb¹ | | FcγRIII | | FcγRIV | |
|---|---|---|---|---|---|---|---|---|
| | K_{D} | Fold² | K_{D} | Fold | K_{D} | Fold | K_{D} | Fold |
| 2372/2373 | 113 | 0.7 | 236 | 0.5 | 88.6 | 0.6 | 54.5 | 2.1 |
| WT | | | | | | | | |
| 2372/2373 | 170 | | 438 | | 143 | | 26 | |
| AF | | | | | | | | |
| Surrogate | 139 | 1.0 | 607 | 1.7 | 101 | 0.9 | 69.1 | 2.5 |
| WT | | | | | | | | |
| Surrogate | 143 | | 357 | | 117 | | 27.4 | |
| AF | | | | | | | | |

¹Low responses due to the low affinity of these interactions significantly reduces curve fitting quality
²Fold = K_{D} 2372/2373 WT/K_{D} 2372/2373 AF

### Example 8—Binding to FcγRIIIa-Expressing Cells of GITR/CTLA-4 Bispecific Antibodies with Afucosylated Fc Domain

To confirm the enhanced binding to FcγRIIIa of the afucosylated GITR/CTLA-4 bispecific antibody, binding to FcγRIIIa-expressing cells was assessed by flow cytometry.

### Material and Methods

Transfected CHO cells stably expressing high levels of FcγRIIIa (V158) (CHO-FcγRIIIa cells) were used. 250,000 cells/well was stained with serially diluted GITR/CTLA-4 bispecific antibodies in FACS buffer (PBS with 0.5% BSA) for 1 h at 4° C. Cells were washed in FACS buffer followed by the addition of a secondary PE-conjugated anti-hFc antibody (Jackson, #109-115-098) diluted 1:100 in FACS buffer. After a 30-min incubation at 4° C., cells were washed twice, resuspended in FACS buffer and analysed on a FACS Verse.

### Results and Conclusions

As expected, an enhanced binding to FcγRIIIa-expressing cells was seen with the afucosylated bispecific antibody compared to the wildtype IgG1 variant (FIG. 8).

### Example 9—Binding of GITR/CTLA-4 Bispecific Antibodies to the C1q Component of Human Complement

In this example, the binding to the C1q component of the human complement system was evaluated using GITR/CTLA-4 bispecific antibodies with wildtype and afucosylated IgG1 format.

### Material and Methods

ELISA plates were coated with human C1q protein (2 μg/ml), 50 μl/well (Calbiochem, #204876). The plates were then washed 3 times with PBST (PBS+0.05% polysorbate 20) and blocked with PBST and 1% BSA for 1 h at room temperature. After 3 washes with PBST, the monoclonal or bispecific antibodies were added at different concentrations and incubated for 2 h at room temperature. The plates were washed as above, and 50 μl sheep anti-human C1q-HRP (BioRad, #2221-5004P) was added at a 1:400 dilution. After 1 h incubation at room temperature, plates were washed 6 times in PBST, followed by the addition of 50 μl peroxidase (Pierce, #37069). Luminescence was measured in a Fluorostar Optima (BMG Labtech).

### Results and Conclusions

As shown in FIG. 9, a similar dose-dependent binding to C1q was seen with wildtype and afucosylated 2372/2373, and the level was on par with the IgG1 isotype control. As expected, no binding was seen with the IgG4 isotype control. Rituximab (Mabthera®), on the other hand, that was included as a positive control due to its ability to bind C1q and mediate complement-mediated lysis, gave a strong signal.

### Example 10—Agonistic Function of Bispecific GITR/CTLA-4 Antibodies

The ability of bispecific GITR/CTLA-4 antibodies to activate T cells expressing GITR in the presence of CTLA-4 was determined. T cell activation with an increase in IFN₇ production was expected in the presence of cross-linking of GITR via the bispecific antibody binding to CTLA-4 coated wells. The aim was to achieve higher efficacy and potency of the bispecific antibodies when CTLA-4 was present as well as higher efficacy than the combination of a GITR monospecific antibody (GITR mAb) and the isotype control coupled to the CTLA-4 binding part (iso/CTLA-4). Furthermore, bispecific antibody 2372/2373 in wildtype and afucosylated format was compared. No change in agonistic function is expected with an afucosylated bispecific antibody format.

### Material and Methods

Human CD3 positive T cells were purified from Ficoll separated PBMCs (obtained from leucocyte filters from the blood bank of the Lund University Hospital) using negative selection (Pan T cell Isolation Kit, human, Miltenyi, 130-096-535). 50 μl of α-CD3 (clone: OKT3, BD, concentration: 3 μg/ml) with or without CTLA-4 (Orencia, 5 μg/ml) diluted in PBS was coated to the surface of a non-tissue cultured treated, U-shaped 96-well plates (Nunc, VWR #738-0147) overnight at 4° C. After washing, T cells were added (100,000 cells/well). Bispecific GITR/CTLA-4 antibodies were added in a serial dilution to the wells and compared at the same molar concentrations to a combination of 2 monospecific controls: 1) GITR mAb, a commercially available monospecific GITR antibody (DT5D3, Miltenyi Biotec) and 2) iso/CTLA-4, an isotype control coupled to the CTLA-4 binding part. CTLA-4 coated wells were compared with non CTLA-4 coated wells. After 72 h of incubation in a moisture chamber at 37° C., 5% CO2, IFNγ and/or IL-2 levels were measured in the supernatant by ELISA.

### Results and Conclusions

The results in FIG. 10 show a dose-dependent agonistic effect of the soluble bispecific antibodies that induce an increase in T cell IFNγ production only when cultured in plates coated with α-CD3 and CTLA-4, while the combination of a monospecific GITR antibody and an isotype control with the CTLA-4 binding part do not. The in vitro assay represents an experimental model of the situation where both GITR and CTLA-4 are relatively overexpressed in the tumour microenvironment. The results thus indicate that the bispecific antibodies have an increased agonistic effect that is dependent on CTLA-4 present in an environment with high levels of activated T cells or Tregs, e.g. the tumour microenvironment, in comparison with monospecific antibodies. Moreover, as shown in FIG. 11, no difference in the agonistic effect of the wildtype and the afucosylated 2372/2373 variant was seen.

### Example 11—Agonistic Function of GITR/CTLA-4 Bispecific Antibodies Upon FcγRIIIa Crosslinking

For many immunomodulatory antibodies, FcγR engagement is critical for their efficacy. In this example, the agonistic activity of bispecific GITR/CTLA-4 antibodies was examined in the presence of FcγRIIIa crosslinking. Due to the enhanced binding to FcγRIIIa of the afucosylated GITR/CTLA-4 bispecific antibody, an increased activation is expected of this variant compared to the wildtype IgG1.

### Material and Methods

The agonistic function of bispecific GITR/CTLA-4 antibodies was tested in a GITR activation assay (GITR Bioassay, Promega, #CS184006), containing Jurkat cells stably expressing GITR and luciferase downstream of a response element. Activation induced by the test antibodies was quantified through the luciferase produced and measured as luminescence. The induction of GITR activation was determined in response to serially diluted GITR/CTLA-4 bispecific antibodies and isotype control in the absence or presence of transfected CHO cells (100,000 cells/well) stably expressing FcγRIIIa (V158). After a 6-h incubation period, Bio-Glo Luciferase Assay Reagent was added, and the luminescence was measured.

### Results and Conclusions

As shown in FIG. 12A, a similar activation is seen with the wildtype and afucosylated 2372/2373 antibody. However, in the presence of FcγRIIIa crosslinking, the GITR activation is higher with the afucosylated bispecific antibody (FIG. 12B).

### Example 12—Ability of GITR/CTLA-4 Bispecific Antibodies to Induce Target-Cell Depletion in an ADCC Reporter Assay

One mode of action of the GITR/CTLA-4 bispecific antibodies is to induce ADCC of target-expressing cells. In the tumor environment, these constitute Tregs that have a high expression of both GITR and CTLA-4. To mimic this milieu, transfected CHO cells with a stable expression of high levels of GITR and CTLA-4 (CHO-GITR^{hi}-CTLA4^{hi} cells) as well as high levels of GITR and low levels of CTLA-4 (CHO-GITR^{hi}-CTLA4^{lo} cells) have been generated. The ability of wildtype and afucosylated GITR/CTLA-4 bispecific antibodies to induce ADCC of target-expressing cells were tested using an ADCC Reporter assay. As afucosylated antibodies have a higher affinity for FcγRIIIa, an enhanced ADCC of this format is expected.

### Material and Methods

A reporter-based system from Promega was used (ADCC Reporter Bioassay Kit, #G7010), containing Jurkat effector cells stably expressing the FcγRIIIa (V158) receptor and an NFAT response element driving the expression of firefly luciferase. Effector cell activation induced by the test antibodies was quantified through the luciferase produced and measured as luminescence. The induction of ADCC in response to serially diluted GITR/CTLA-4 bispecific antibodies, a mix of the monoclonal counterparts (iso/CTLA-4+αGITR mAb) and isotype control was determined using CHO-GITR^{hi}-CTLA4^{hi} and CHO-GITR/CTLA4^{lo} cells as target cells. The effector:target cell ratio was 5:1. After a 6-h incubation period, Bio-Glo Luciferase Assay Reagent was added, and the luminescence was measured.

### Results and Conclusions

As shown in FIG. 13 using CHO-GITR^{hi}-CTLA4^{lo} cells as target cells, a superior effect of the GITR/CTLA-4 bispecific antibody is seen compared to the combination of the two monoclonal counterparts at equal molar concentrations. No effect was seen with the isotype control. Moreover, a superior FcγRIIIa activation as a model of ADCC was seen with the afucosylated bispecific antibody using both CHO-GITR^{hi}-CTLA^{lo} cells (FIG. 14A) and CHO-GITR^{hi}-CTLA4^{hi} cells as target cells (FIG. 14B).

### Example 13—Ability of GITR/CTLA-4 Bispecific Antibodies to Induce PBMC-Mediated Lysis of Target-Expressing Cells

### Material and Methods

In order to determine the ability of GITR/CTLA-4 bispecific antibodies to induce depletion of target-expressing cells, the level of ADCC mediated by primary PBMC as effector cells was investigated. Transfected CHO cells stably expressing high levels of GITR and CTLA-4 (CHO-GITR^{hi}-CTLA4^{hi} cells) were used as target cells. The LDH Cytotoxicity Assay (Pierce, #88953) was used to assess cell lysis. PBMC was purified from leukocyte filters from healthy donors. Effector cells and target cells were incubated at an effector:target cell ratio of 50:1 with serially diluted GITR/CTLA-4 bispecific antibodies or isotype control for 4 h. Thereafter, the level of LDH in the supernatants was measured.

### Results and Conclusions

As shown in FIG. 15, a superior depletion of target-expressing cells was seen with the afucosylated bispecific antibody over the wildtype IgG1 variant.

### Example 14—Ability of GITR/CTLA-4 Bispecific Antibodies to Deplete Primary Tregs

The in vitro ADCC activity of the GITR/CTLA-4 bispecific antibodies was assessed using an ADCC Reporter assay with Tregs that express GITR and CTLA-4 as target cells.

### Material and Methods

An ADCC Reporter assay (Promega, #G7010) was used containing effector cells stably expressing the FcγRIIIa (V158) receptor. CD4⁺CD25⁺CD127^{low} Tregs were isolated by negative selection using the EasySep™ Human CD4⁺CD127^{low}CD25⁺ Regulatory T Cell Isolation Kit (Stemcell Technologies, #18063) and used as target cells. Tregs were either used fresh in the ADCC Reporter assay, or after activation for 48 h in the presence of Human T-Activator CD3/CD28 Dynabeads (Gibco, #11131D) to up-regulate the expression of GITR and CTLA-4. The induction of ADCC was assessed in response to serially diluted GITR/CTLA-4 bispecific antibodies. Effector and target cells were cultured at a 5:1 ratio for a period of 6 or 18 h. The expression of GITR and CTLA-4 was determined before and after culture by flow cytometry.

### Results and Conclusions

The GITR/CTLA-4 bispecific antibodies did not mediate ADCC in fresh Tregs (FIG. 16A). However, after activation for 48 h with αCD3/CD28 beads, ADCC was induced. The induction was markedly higher with the afucosylated variant compared to the wildtype IgG1 format (FIG. 16B). The results correlated with the expression levels of GITR and CTLA-4. Fresh PBMC and Tregs expressed low levels of GITR and CTLA-4, whereas the levels were clearly up-regulated after in vitro activation (FIG. 16C).

### Example 15—Ability of GITR/CTLA-4 Bispecific Antibodies to Induce Cytokine Release

Cytokine release syndrome is a potentially life-threatening toxicity that has been observed in cancer immunotherapy with antibodies. Here we have compared a wildtype and an afucosylated GITR/CTLA-bispecific antibody for their ability to induce cytokine release in a whole blood and a PBMC-based cytokine release assay.

### Material and Methods

The ability of wildtype and an afucosylated GITR/CTLA-bispecific antibody 2372/2373 to induce cytokine release was tested in a whole blood and a PBMC cytokine release assay (CRA) at KWS Biotest (Bristol, UK). Alemtuzumab, Muromonab and Ancell anti-CD28 (ANC28.1) were included as positive controls, and non-specific IgG1, IgG4 and IgG2a as negative controls. All antibodies were tested at 0.1, 1 and 10 μg/well.

Whole blood was taken from 4 healthy donors. Test antibodies and controls were added to the blood in duplicates. Cytokine production was assessed after 48 hours of culture. PBMC was separated from whole blood samples collected from 3 healthy donors. Test antibodies and controls were immobilized to the wells before the addition of PBMC. Uncoated wells acted as negative controls and each condition was tested in duplicate. Cytokine production was assessed after a 72-h culture period. For both assays, the Proinflammatory Panel 1 (human) was used for the quantitative determination of IFNγ, IL-1β, IL-2, IL-4, IL-6, IL-8, IL-10, IL-12p70, IL-13, and TNFα in the culture supernatants using the Luminex platform.

Data analysis was carried out using linear regression followed by a one-way ANOVA with Tukey's post-hoc test to compare slopes amongst different treatment groups for each cytokine. Linear regression analysis gave a slope equal to 0 for some of the cytokines which did not allow us to perform a one-way ANOVA. A 2-way ANOVA followed by Tukey's post-hoc test was used to compare the effect of treatments at different concentrations on each cytokine for the whole blood and wet coat assays.

### Results and Conclusions

For all donors tested, unstimulated cells showed the expected levels of background cytokine release in both CRA formats. The positive control antibodies resulted in robust cytokine responses, with levels as expected within each cytokine release assay format. In comparison with immobilized CRA formats, whole blood CRAs typically result in higher donor variability.

Neither of the GITR/CTLA-4 bispecific antibodies induced IL-1β, IL-2, IL-4, IL-6, IL-8, IL-10, IL-12p70, IL-13, TNFα and IFNγ above levels induced by the IgG1 isotype control in either assay. High levels of IL-8 were induced in the absence of antibody in both assays and this is not unexpected for this cytokine. A slight raise in IL-8 levels in positive control cultures suggests that stimulation was able to raise IL-8 production levels above background.

In both assays, neither the wildtype nor the afucosylated GITR/CTLA-4 bispecific antibody induced cytokine secretion above the levels induced by the IgG1 isotype control.

### Example 16—Agonistic Function of Murine Surrogate Bispecific GITR/CTLA-4 Antibodies

In order to study bispecific antibodies in in vivo models, surrogate bispecific antibodies targeting murine GITR/CTLA-4 were generated using human IgG1 format in a wildtype (2776/2777) and afucosylated variant (2776/2777 AF). To assess the ability of the surrogate bispecific GITR/CTLA-4 antibodies to activate murine T cells, splenocyte assays were utilized determining T cell activation in form of IFN-γ production. Both bispecific variants were able to activate T cells, and as expected, no differences in activation levels were observed between the wildtype and afucosylated variant.

### Material and Methods

Murine CD3⁺ T cells isolated from the spleens of C57BL6 mice (Miltenyi, Pan-T Isolation kit II) were added to a 96-well plate coated with αCD3 (BD, 0.8 μg/mL) and CTLA-4 (Orencia, 5 μg/ml). Bispecific GITR/CTLA-4 antibodies were added in a serial dilution and compared to isotype or isotype/CTLA-4 control. T cell activation in form of IFN-γ release was measured after 48 h by ELISA.

### Results and Conclusions

The agonistic effects of surrogate bispecific antibodies were investigated in splenocyte assays. Both the wildtype and afucosylated variant demonstrated agonistic T cell activation and induction of dose-dependent IFN-γ release (FIG. 17). This T cell activation was not seen in wells without CTLA-4 coating, or in wells containing the isotype controls. As expected, no differences in the agonistic effects were seen between the wildtype and afucosylated 2776/2777 variants.

### Example 17—Ability of Murine Surrogate GITR/CTLA-4 Bispecific Antibodies to Induce ADCC in a Reporter Assay

In the tumor environment, Tregs have a high expression of both GITR and CTLA-4. GITR/CTLA-4 bispecific antibodies are expected to induce ADCC of target-expressing cells, especially in the tumor environment. The ability of murine bispecific surrogates as wildtype and afucosylated variants to induce ADCC was examined using an ADCC reporter assay specific for murine FcγRIV. Both variants of bispecific antibodies demonstrated activation of the reporter cells. However, as the afucosylated antibody has a higher affinity for murine FcγRIV than the wildtype antibody, the afucosylated variant demonstrated enhanced ADCC induction.

### Material and Methods

A reporter-based system (Promega ADCC Reporter Bioassay Kit), for mFcγRIV receptor was used to determinate ADCC in response to GITR/CTLA-4 bispecific antibodies or to isotype controls using mGITR coated wells. Effector cells were added at fixed concentration and ADCC was induced for 6 h.

### Results and Conclusions

The ability of the wildtype and afucosylated variants of the GITR/CTLA-4 bispecific surrogate antibodies to induce ADCC was investigated using ADCC reporter assay. Both variants were able to activate the murine specific FcγRIV reporter cells which serving as indication for ADCC induction (FIG. 18). In consistency with afucosylated antibody having higher affinity for murine FcγRIV than the wildtype antibody, a superior ADCC induction was detected with the afucosylated bispecific antibody variant over the wildtype. These findings demonstrate the relevant mimicry of the murine system compared with human, providing a model to study ADCC effects and the mode of action, despite the fact that mice and human differ in their Fc receptor functions.

### Example 18—Anti-Tumor Efficacy of Murine Surrogate GITR/CTLA-4 Bispecific Antibodies in CT26 Colon Carcinoma Model

The anti-tumor effects of the surrogate bispecific antibodies were examined against CT26 colon carcinoma model using BalbC mice. Both wildtype and afucosylated antibody variants demonstrated statistically significant anti-tumor efficacy in form of tumor volume inhibition and increased survival.

### Material and Methods

Female BalbC mice from Janvier, France, 7-8 w old, were used in the experiments. All experiments were approved by the Malmo/Lund Ethical Committee.

CT26 colon carcinoma growing in log phase was injected subcutaneously (0.1×10⁶ cells) on day 0 and mice were treated with 2776/2777 or 2776/2777 AF (200 μg) intraperitoneally on days 7, 10 and 13. Rat anti-mouse GITR antibody DTA-1 (in molar equivalent, BioXcell, US) was used as a positive control. The tumors were measured three times per week with a caliper and the tumor volume was calculated using formula ((width/2)×(length/2)×(height/2)×pi×(4/3)). The statistical analysis was done using GraphPad Prism program, Mann-Whitney non-parametric 2-tail test for tumor growth and Kaplan-Meyer survival, log-rank (Mantel-Cox) for survival.

### Results and Conclusions

The anti-tumor efficacy of the bispecific GITR/CTLA-4 surrogate 2776/2777 was investigated in BalbC mice using CT26 colon carcinoma model. The wildtype variant 2776/2777 demonstrated statistically significant anti-tumor efficacy compared to vehicle in form of tumor volume inhibition, p=0.0002 (FIG. 19A). This anti-tumor efficacy was superior to the positive control antibody DTA-1.

Similarly, the treatment with afucosylated variant 2776/2777AF significantly increased survival of the mice compared to vehicle treatment (p=0.0029), and approximately 30% of mice were cured from established tumors by the treatment (FIG. 19B).

### Example 19—Anti-Tumor Efficacy of Murine Surrogate GITR/CTLA-4 Bispecific Antibodies in MC38 Colon Carcinoma Model

The anti-tumor effects of the surrogate bispecific antibodies were examined against MC38 colon carcinoma model using C57BL6 mice. Both wildtype and afucosylated bispecific antibodies demonstrated statistically significant anti-tumor efficacy in form of tumor volume inhibition and increased survival.

### Material and Methods

Female C57BL6 mice from Janvier, France, 7-8w old, were used in the experiments. All experiments were approved by the Malmo/Lund Ethical Committee.

MC38 colon carcinoma growing in log phase was injected subcutaneously (1×10⁶ cells) on day 0 and mice were treated with 2776/2777 or 2776/2777 AF (200 μg) intraperitoneally on days 7, 10 and 13 after the tumors were established. The tumors were measured three times a week with a caliper and tumor volume was calculated using formula ((w/2)×(l/2)×(h/2)×pi×(4/3)). The statistical analysis was done using GraphPad Prism program, Mann-Whitney, non-parametric 2-tail test for tumor growth and Kaplan-Meyer survival, log-rank (Mantel-Cox) for survival.

### Results and Conclusions

The anti-tumor efficacy of the bispecific GITR/CTLA-4 surrogate 2776/2777 as wildtype or afucosylated variant was investigated in C57BL6 mice using MC38 colon carcinoma model. 2776/2777 demonstrated statistically significant anti-tumor efficacy compared to vehicle in form of tumor volume inhibition, p=0.0006 (FIG. 20A). Similarly, the afucosylated 2776/2777 AF treatment significantly increased survival of the mice (p=0.001) and approximately 30% of mice were complete responders (FIG. 20B).

### Example 20—Anti-Tumor Efficacy in Form of CD8/Treg Ratio after Treatment with Murine Surrogate GITR/CTLA-4 Bispecific Antibodies in MC38 Colon Carcinoma Model

The anti-tumor effects of the surrogate bispecific antibodies in form of intratumoral CD8/Treg ratio were examined in MC38 colon carcinoma model using C57BL6 mice. Both the wildtype 2776/2777 and afucosylated 2776/2777 AF bispecific antibody demonstrated depletion of regulatory T cells, and as expected, the afucosylated variant demonstrated superior depletion over the wildtype variant.

### Material and Methods

Female C57BL6 mice from Janvier, France, 7-8 weeks old, were used in the experiments. All experiments were approved by the Malmo/Lund Ethical Committee.

MC38 colon carcinoma growing in log phase was injected subcutaneously (1×10⁶ cells) on day 0 and mice were treated with 2776/2777 or 2776/2777 AF (200 μg) intraperitoneally on days 10, 13 and 16. Twenty-four hours after the last injection, the tumors and spleens were harvested, stained for viability marker as well as lineage markers (CD11b, CD19, MHCII and NK1.1), CD45, CD3, CD4, CD8, CD25, Foxp3, and analyzed using flow cytometry. Regulatory T cells were gated as live/single cell/CD45/CD3/CD4/Foxp3/CD25.

### Results

The pharmacodynamic effects of the bispecific GITR/CTLA-4 antibodies were investigated in C57BL6 mice using the MC38 colon carcinoma model. The results in FIG. 21 demonstrate intratumoral Treg depletion by both bispecific antibody variants, however the afucosylated variant, as expected, demonstrated superior activity over the wildtype (FIG. 21A). This effect was further seen in the CD8/Treg ratio (FIG. 21B). No changes in the CD8/Treg ratio can be seen in the spleen, indicating that the effects of the bispecific antibodies are mainly directed to the tumor microenvironment (FIG. 21C).

### Example 21—Anti-Tumor Efficacy of Human GITR/CTLA-4 Bispecific Antibodies in Human Plasmacytoma Model

The anti-tumor effects of the human bispecific GITR/CTLA-4 bispecific antibodies as wildtype and afucosylated variants were investigated using immunodeficient mice humanized by administering hPBMC in a subcutaneous tumor model of RPMI-8226 plasmacytoma. Both bispecific variants (2372/2373 and 2372/2373 AF) demonstrated statistically significant anti-tumor effects with and without human PBMC as effector cells, indicating that the bispecific antibodies have potential to have both direct and indirect anti-tumor efficacies. In addition, the afucosylated antibody demonstrated superior anti-tumor efficacy over the wildtype variant in this model.

Material and Methods Female SCID-Beige mice 7-8 w old from Taconic, Denmark, were used in the experiments. All experiments were approved by the Malmo/Lund Ethical Committee.

Leukocyte filters were obtained from Lund University Hospital and hPBMC were isolated by Ficoll centrifugation. RPMI-8226 plasmacytoma growing in log phase was injected subcutaneously (10×10⁶ cells) on day 0, hPBMC (5×10⁶ cells) were injected intraperitoneally on day 5 and the antibody treatments (app 500 nmol) were done on days 5, 11 and 18. The tumor volume was measured three times a week with a caliper and tumor volume was calculated using formula ((w/2)×(l/2)×(h/2)×pi×(4/3)). The statistical analysis was done using GraphPad Prism program, Mann-Whitney, non-parametric 2-tail test for tumor growth.

### Results

The anti-tumor efficacy of GITR/CTLA-4 antibodies was investigated in hPBMC humanized mouse models using RPMI-8226 plasmacytoma model. The results in FIG. 22 and Table 5 show that both wildtype and afucosylated variants of the bispecific antibody demonstrated statistically significant anti-tumor efficacy in form of tumor growth inhibition in the presence of hPBMC (FIG. 22A), and without hPBMC (FIG. 22B). The afucosylated variant (2372/2373 AF) demonstrated statistically significant superiority over the wildtype variant (2372/2373) in form of tumor volume inhibition. The percentage of tumor volume inhibition compared to vehicle can be found in Table 5.

TABLE 5
Anti-tumor activity in GITR/CTLA-4 treated tumors

| With PBMC | | | | | | Without PBMC | | | | | |
|---|---|---|---|---|---|---|---|---|---|---|---|
| | 2372/2373 | | 2372/2373 AF | | 2372/2373 | | 2372/2373 | | 2372/2373 AF | | 2372/2373 |
| | vs Vehicle | | vs vehicle | | vs | | vs Vehicle | | vs vehicle | | vs |
| | % tumor | | % tumor | | 2372/2373 | | % tumor | | % tumor | | 2372/2373 |
| | volume | | volume | | AF | | volume | | volume | | AF |
| Day | inhibition | p-value | inhibition | p-value | p-value | Day | inhibition | p-value | inhibition | p-value | p-value |
| D29 | 100.0 | <0.0001 | 100.0 | <0.0001 | >0.9999 | D29 | 100.0 | <0.0001 | 100.0 | <0.0001 | >0.9999 |
| D32 | 98.8 | <0.0001 | 100.0 | <0.0001 | >0.9999 | D32 | 93.2 | <0.0001 | 100.0 | <0.0001 | 0.0108 |
| D34 | 95.7 | <0.0001 | 100.0 | <0.0001 | 0.4737 | D34 | 86.6 | <0.0001 | 100.0 | <0.0001 | 0.0108 |
| D36 | 93.9 | <0.0001 | 100.0 | <0.0001 | 0.4737 | D36 | 86.4 | <0.0001 | 100.0 | <0.0001 | 0.0108 |
| D39 | 87.4 | <0.0001 | 100.0 | <0.0001 | 0.2105 | D39 | 78.9 | <0.0001 | 100.0 | <0.0001 | 0.0108 |
| D41 | 87.2 | <0.0001 | 100.0 | <0.0001 | 0.2105 | D41 | 79.2 | <0.0001 | 100.0 | <0.0001 | 0.0007 |
| D43 | 90.6 | 0.0002 | 100.0 | <0.0001 | 0.2105 | D43 | 81.1 | <0.0001 | 100.0 | <0.0001 | 0.0007 |
| D46 | 76.2 | 0.0037 | 100.0 | <0.0001 | 0.0325 | D46 | 76.1 | <0.0001 | 100.0 | <0.0001 | 0.0007 |
| D48 | 77.3 | 0.0037 | 100.0 | <0.0001 | 0.0108 | D48 | 72.9 | 0.0002 | 100.0 | <0.0001 | 0.0007 |
| D50 | 71.0 | 0.0037 | 100.0 | <0.0001 | 0.0108 | D50 | 72.1 | 0.0002 | 100.0 | <0.0001 | 0.0007 |
| D53 | 66.9 | 0.0083 | 100.0 | <0.0001 | 0.0108 | D53 | N.A | N.A | N.A | N.A | <0.0001 |
| D55 | 68.4 | 0.0107 | 100.0 | <0.0001 | 0.0108 | D55 | N.A | N.A | N.A | N.A | <0.0001 |
| D57 | 51.9 | 0.0329 | 100.0 | <0.0001 | 0.0108 | D57 | N.A | N.A | N.A | N.A | <0.0001 |

### REFERENCES

1: Baessler T, Charton J E, Schmiedel B J, Grünebach F, Krusch M, Wacker A, Rammensee H G, Salih H R. CD137 ligand mediates opposite effects in human and mouse N K cells and impairs N K-cell reactivity against human acute myeloid leukemia cells. Blood. 2010 Apr. 15; 115(15):3058-69. doi: 10.1182/blood-2009-06-227934. Erratum in: Blood. 2010 Dec. 23; 116(26):6152. PubMed PMID: 20008791.
2: Bulliard Y, Jolicoeur R, Windman M, Rue S M, Ettenberg S, Knee D A, Wilson N S, Dranoff G, Brogdon J L. Activating Fc γ receptors contribute to the antitumour activities of immunoregulatory receptor-targeting antibodies. J Exp Med. 2013 Aug. 26; 210(9):1685-93. doi: 10.1084/jem.20130573. PubMed PMID: 23897982; PubMed Central PMCID: PMC3754864.
3: Cohen A D, Schaer D A, Liu C, Li Y, Hirschhorn-Cymmerman D, Kim S C, Diab A, Rizzuto G, Duan F, Perales M A, Merghoub T, Houghton A N, Wolchok J D. Agonist anti-GITR monoclonal antibody induces melanoma tumour immunity in mice by altering regulatory T cell stability and intra-tumour accumulation. PLoS One. 2010 May 3; 5(5):e10436. doi: 10.1371/journal.pone.0010436. PubMed PMID: 20454651; PubMed Central PMCID: PMC2862699.
4: Fournier P, Schirrmacher V. Bispecific antibodies and trispecific immunocytokines for targeting the immune system against cancer: preparing for the future. BioDrugs. 2013 February; 27(1):35-53. doi: 10.1007/s40259-012-0008-z. Review. PubMed PMID: 23329400.
5: Fransen M F, van der Sluis T C, Ossendorp F, Arens R, Melief C J. Controlled local delivery of CTLA-4 blocking antibody induces CD8+ T-cell-dependent tumour eradication and decreases risk of toxic side effects. Clin Cancer Res. 2013 Oct. 1; 19(19):5381-9. doi: 10.1158/1078-0432.CCR-12-0781. PubMed PMID: 23788581.
6: Furness A J, Vargas F A, Peggs K S, Quezada S A. Impact of tumour microenvironment and Fc receptors on the activity of immunomodulatory antibodies. Trends Immunol. 2014 July; 35(7):290-8. doi: 10.1016/j.it.2014.05.002. Review. PubMed PMID: 24953012.
7: Kanamaru F, Youngnak P, Hashiguchi M, Nishioka T, Takahashi T, Sakaguchi S, Ishikawa I, Azuma M. Costimulation via glucocorticoid-induced TNF receptor in both conventional and CD25+ regulatory CD4+ T cells. J Immunol. 2004 Jun. 15; 172(12):7306-14. PubMed PMID: 15187106.
8: Levings M K, Sangregorio R, Sartirana C, Moschin A L, Battaglia M, Orban P C, Roncarolo M G. Human CD25+CD4+T suppressor cell clones produce transforming growth factor beta, but not interleukin 10, and are distinct from type 1 T regulatory cells. J Exp Med. 2002 Nov. 18; 196(10):1335-46. PubMed PMID: 12438424; PubMed Central PMCID: PMC2193983.
9: May C, Sapra P, Gerber H P. Advances in bispecific biotherapeutics for the treatment of cancer. Biochem Pharmacol. 2012 Nov. 1; 84(9):1105-12. doi: 10.1016/j.bcp.2012.07.011. Review. PubMed PMID: 22858161.
10: McHugh R S, Whitters M J, Piccirillo C A, Young D A, Shevach E M, Collins M, Byrne M C. CD4(+)CD25(+) immunoregulatory T cells: gene expression analysis reveals a functional role for the glucocorticoid-induced TNF receptor. Immunity. 2002 February; 16(2):311-23. PubMed PMID: 11869690.
11: Nocentini G, Giunchi L, Ronchetti S, Krausz L T, Bartoli A, Moraca R, Migliorati G, Riccardi C. A new member of the tumour necrosis factor/nerve growth factor receptor family inhibits T cell receptor-induced apoptosis. Proc Natl Acad Sci USA. 1997 Jun. 10; 94(12):6216-21. PubMed PMID: 9177197; PubMed Central PMCID: PMC21029.
12: Pedroza-Gonzalez A, Zhou G, Singh S P, Boor P P, Pan Q, Grunhagen D, de Jonge J, Tran T K, Verhoef C, IJzermans J N, Janssen H, Biermann K, Kwekkeboom J, Sprengers D. GITR engagement in combination with CTLA-4 blockade completely abrogates immunosuppression mediated by human liver tumour-derived regulatory T cells ex vivo. Oncoimmunology. 2015 May 29; 4(12):e1051297. PubMed PMID: 26587321; PubMed Central PMCID: PMC4635937.
13: Pruitt S K, Boczkowski D, de Rosa N, Haley N R, Morse M A, Tyler D S, Dannull J, Nair S. Enhancement of anti-tumour immunity through local modulation of CTLA-4 and GITR by dendritic cells. Eur J Immunol. 2011 December; 41(12):3553-63. doi: 10.1002/eji.201141383. PubMed PMID: 22028176; PubMed Central PMCID: PMC3594439.
14: Ronchetti S, Ricci E, Petrillo M G, Cari L, Migliorati G, Nocentini G, Riccardi C. Glucocorticoid-induced tumour necrosis factor receptor-related protein: a key marker of functional regulatory T cells. J Immunol Res. 2015; 2015:171520. doi: 10.1155/2015/171520. Review. PubMed PMID: 25961057; PubMed Central PMCID: PMC4413981.
15: Shimizu J, Yamazaki S, Takahashi T, Ishida Y, Sakaguchi S. Stimulation of CD25(+)CD4(+) regulatory T cells through GITR breaks immunological self-tolerance. Nat Immunol. 2002 February; 3(2):135-42. PubMed PMID: 11812990.
16: Walker L S, Sansom D M. The emerging role of CTLA-4 as a cell-extrinsic regulator of T cell responses. Nat Rev Immunol. 2011 Nov. 25; 11(12):852-63. doi: 10.1038/nri3108. PubMed PMID: 22116087.

1. A multispecific polypeptide comprising a first binding domain, designated B1, which is capable of specifically binding to GITR, and a second binding domain, designated B2, which is capable of specifically binding to CTLA-4.

2. A polypeptide according to claim 1, wherein the first and/or second binding domains are selected from the group consisting of: antibodies or antigen-binding fragments thereof.

3. A polypeptide according to claim 2 wherein the antigen-binding fragment is selected from the group consisting of: an Fv fragment (such as a single chain Fv fragment, or a disulphide-bonded Fv fragment), a Fab-like fragment (such as a Fab fragment; a Fab′ fragment or an F(ab)₂ fragment) and domain antibodies.

4. A polypeptide according to any one of the preceding claims wherein the polypeptide is a bispecific antibody.

5. A polypeptide according to any one of the preceding claims wherein:
(a) B1 comprises or consists of an IgG1 antibody and B2 comprises or consists of an scFv, or vice versa; or
(b) B1 comprises or consists of at least one scFv and B2 comprises or consists of at least one scFv.

6. A polypeptide according to any one of claims 1 to 3 wherein the first and/or second binding domains is non-antibody polypeptide.

7. A polypeptide according to claim 6 wherein B1 comprises or consists of an IgG1 antibody and B2 comprises or consists of a non-immunoglobulin polypeptide, or vice versa.

8. A polypeptide according to claim 6 or 7 wherein B2 comprises or consists of a CD86 domain or variant thereof capable of binding to CTLA-4.

9. A polypeptide according to any one of the preceding claims in which B1 comprises at least one heavy chain (H) and/or at least one light chain (L) and B2 is attached to said at least one heavy chain (H) or least one light chain (L).

10. A polypeptide according to claim 9 in which B1 comprises:
(a) at least one heavy chain (H) and at least one light chain (L) and B2 is attached to either the heavy chain or the light chain; or
(b) two identical heavy chains (H) and two identical light chains (L) and B2 is attached to both heavy chains or to both light chains.

11. A polypeptide according to claim 9 or 10 which comprises or consists of a polypeptide chain arranged according to any one of the following formulae, written in the direction N-C:
L-(X)n-B2; (a)
B2-(X)n-L; (b)
B2-(X)n-H; or (c)
H-(X)n-B2; (d)
wherein X is a linker and n is 0 or 1.

12. A polypeptide according to claim 12, wherein X is a peptide with the amino acid sequence SGGGGSGGGGS (SEQ ID NO: 47), SGGGGSGGGGSAP (SEQ ID NO: 48), NFSQP (SEQ ID NO:49), KRTVA (SEQ ID NO: 50), GGGGSGGGGSGGGGS (SEQ ID NO: 51) or (SG)m, where m=1 to 7.

13. A polypeptide according to any one of the preceding claims comprising a human Fc region or a variant of a said region, where the region is an IgG1, IgG2, IgG3 or IgG4 region, preferably an IgG1 or IgG4 region.

14. A polypeptide according to claim 13 wherein the Fc region is a naturally occurring (i.e. wildtype) human Fc region.

15. A polypeptide according to claim 13 wherein the Fc region is a non-naturally occurring (e.g. mutated) human Fc region.

16. A polypeptide according to any one of claims 13 to 15 wherein the Fc region is afucosylated.

17. A polypeptide according to any of the preceding claims, wherein the polypeptide is capable of inducing antibody dependent cell cytotoxicity (ADCC), antibody-dependent cellular phagocytosis (ADCP), complement-dependent cytotoxicity (CDC), and/or apoptosis.

18. A polypeptide according to any of the preceding claims, wherein the polypeptide is capable of inducing tumour immunity.

19. A polypeptide according to any one of the preceding claims, which binds to human GITR with a Kd of less than 10×10⁻⁹M, 4×10⁻⁹M, or 1×10⁻⁹M and/or which binds to human CTLA-4 with a Kd value which is less than 60×10⁻⁹M, 25×10⁻⁹M, or 10×10⁻⁹M.

20. A polypeptide according to any one of the preceding claims, which induces an increase in the activity of an effector T cell, optionally wherein said increase is at least 1.5-fold, 4.5-fold or 7-fold higher than the increase in activity of an effector T cell induced by a combination of the first and second binding domains administered to the T cell as separate molecules.

21. A polypeptide according to any one of the preceding claims, wherein the polypeptide is capable of:
i) Killing GITR expressing tumour cells; and
ii) Activating the immune system via activation of effector T cells

22. A polypeptide according to claim 20, wherein said increase in T cell activity is an increase in proliferation and/or IFNγ or IL-2 production by the T cell.

23. A polypeptide according to any of the preceding claims wherein B1 is an antibody, or antigen binding fragment thereof, specific for GITR; and B2 is a polypeptide binding domain specific for CTLA-4, which comprises or consists of:
(a) the amino acid sequence of SEQ ID NO: 3; or
(b) an amino acid sequence in which at least one amino acid is changed when compared to the amino acid sequence of SEQ ID NO: 3 provided that said binding domain binds to human CTLA-4 with higher affinity than wild-type human CD86.

24. A polypeptide according to any one of claims 13 to 20, wherein 1, 2, 3, 4, 5, 6, 7, 8, 9 or 10 amino acids in said amino acid sequence of B2 (ii) are substituted when compared to the amino acid sequence of SEQ ID NO: 3; optionally wherein there are no insertions or deletions compared to the amino acid sequence of SEQ ID NO: 3.

25. A polypeptide according to claim 23, wherein at least one of said amino acid substitutions in said amino acid sequence of B2 is at position 122, and optionally wherein said amino acid sequence is also substituted in at least one of positions 107, 121 and 125.

26. A polypeptide according to any one of the preceding claims wherein said amino acid sequence of B2 comprises or consists of an amino acid sequence selected from any one of SEQ ID NOs 6 to 24.

27. A polypeptide according to any one of the preceding claims wherein the GITR binding domain (B1) is capable of competitively inhibiting the binding to human GITR of an antibody comprising a light chain variable region amino acid sequence selected from the group consisting of SEQ ID NOs: 61, 63, 65 and 67 and a heavy chain variable region amino acid sequence selected from the group consisting of SEQ ID NOs: 52, 54, 56 and 58.

28. A polypeptide according to claim 26 wherein B1 comprises a light chain variable region amino acid sequence comprising the CDRs of SEQ ID NOs: 88, 89 and 90 and/or a heavy chain variable region amino acid sequence comprising the CDRs of SEQ ID NOs: 76, 77 and 78.

29. A polypeptide according to claim 26 wherein B1 comprises a light chain variable region amino acid sequence comprising the CDRs of SEQ ID NOs: 91, 92 and 93 and/or a heavy chain variable region amino acid sequence comprising the CDRs of SEQ ID NOs: 79, 80 and 81.

30. A polypeptide according to claim 26 wherein B1 comprises a light chain variable region amino acid sequence comprising the CDRs of SEQ ID NOs: 94, 89 and 95 and/or a heavy chain variable region amino acid sequence comprising the CDRs of SEQ ID NOs: 82, 83 and 84.

31. A polypeptide according to claim 26 wherein B1 comprises a light chain variable region amino acid sequence comprising the CDRs of SEQ ID NOs: 94, 89 and 96 and/or a heavy chain variable region amino acid sequence comprising the CDRs of SEQ ID NOs: 85, 86 and 87.

32. A polypeptide according to any one of the preceding claims wherein the GITR binding domain (B1) comprises a light chain variable region amino acid sequence selected from the group consisting of SEQ ID NOs: 61, 63, 65 and 67 and/or a heavy chain variable region amino acid sequence selected from the group consisting of SEQ ID NOs: 52, 54, 56 and 58.

33. A polypeptide according to claim 31 wherein B1 comprises a light chain variable region amino acid sequence of SEQ ID NO: 61 and/or a heavy chain variable region amino acid sequence of SEQ ID NO: 52.

34. A polypeptide according to claim 31 wherein B1 comprises a light chain variable region amino acid sequence of SEQ ID NO: 63 and/or a heavy chain variable region amino acid sequence of SEQ ID NO: 54.

35. A polypeptide according to claim 31 wherein B1 comprises a light chain variable region amino acid sequence of SEQ ID NO: 65 and/or a heavy chain variable region amino acid sequence of SEQ ID NO: 56.

36. A polypeptide according to claim 31 wherein B1 comprises a light chain variable region amino acid sequence of SEQ ID NO: 67 and/or a heavy chain variable region amino acid sequence of SEQ ID NO: 58.

37. A polypeptide according to any one of claims 31 to 35 wherein B1 comprises or consists of:
(a) a light chain variable region amino acid sequence of SEQ ID NO: 61 and a heavy chain variable region amino acid sequence of SEQ ID NO: 52;
(b) a light chain variable region amino acid sequence of SEQ ID NO: 63 and a heavy chain variable region amino acid sequence of SEQ ID NO: 54;
(c) a light chain variable region amino acid sequence of SEQ ID NO: 65 and a heavy chain variable region amino acid sequence of SEQ ID NO: 56; or
(d) a light chain variable region amino acid sequence of SEQ ID NO: 67 and a heavy chain variable region amino acid sequence of SEQ ID NO: 58.

38. A polypeptide according to any one of the preceding claims, wherein the GITR binding domain comprises a human Fc region or a variant of a said region, where the region is an IgG1, IgG2, IgG3 or IgG4 region, preferably an IgG1 or IgG4 region.

39. A polypeptide according to any of the preceding claims comprising or consisting of:
(a) a light chain amino acid sequence selected from SEQ ID NOs: 69, 71, 73 and 75; and
(b) a heavy chain variable region amino acid sequence selected from the group consisting of 52, 54, 56 and 58.

40. A polypeptide according to any of the preceding claims comprising or consisting of the amino acid sequences:
(a) SEQ ID NOs: 52 and 69; or
(b) SEQ ID NOs: 54 and 71; or
(c) SEQ ID NO: 56 and 73; or
(d) SEQ ID NOs: 58 and 75.

41. A polypeptide according to any of the preceding claims further comprising at least one further binding domain.

42. A polypeptide according to claim 40 wherein the at least one further binding domain is an antigen-binding fragment selected from the group consisting of: an Fv fragment (such as a single chain Fv fragment, or a disulphide-bonded Fv fragment), a Fab-like fragment (such as a Fab fragment; a Fab′ fragment or a F(ab)₂ fragment) and domain antibodies

43. A polypeptide according to claim 40 or 41 wherein the at least one further binding domain.

44. A polypeptide according to any one of the preceding claims further comprising an additional therapeutic moiety.

45. A composition comprising a bispecific polypeptide according to any one of claims 1 to 38 and at least one pharmaceutically acceptable diluent or carrier.

46. An antibody specific for GITR which is as defined in any one of claims 26 to 37.

47. A polynucleotide encoding a bispecific polypeptide according to any one of claims 1 to 42, or a component polypeptide chain thereof.

48. A bispecific polypeptide according to any one of the preceding claims for use in a method for treating or preventing a neoplastic disease or condition in an individual.

49. A bispecific polypeptide according to claim 48 wherein the disease or condition is a cancer.

50. A bispecific polypeptide according to claim 49 wherein the cancer is selected from the groups consisting of prostate cancer, breast cancer, colorectal cancer, pancreatic cancer, ovarian cancer, lung cancer, cervical cancer, rhabdomyosarcoma, neuroblastoma, multiple myeloma, leukemia, acute lymphoblastic leukemia, melanoma, bladder cancer, gastric cancer, head and neck cancer, liver cancer, skin cancer, lymphoma and glioblastoma.

51. Use of a bispecific polypeptide according to any one of claims 1 to 43 in the preparation of a medicament for treating or preventing a neoplastic disease or condition in an individual.

52. A method of treating or preventing a neoplastic disease or condition in an individual, the method comprising administering to an individual a bispecific polypeptide according to any one of claims 1 to 43.

53. A method according to claim 51, wherein the method comprises administering the bispecific antibody systemically or locally, such as at the site of a tumour or into a tumour draining lymph node.

54. A bispecific polypeptide substantially as herein described with reference to the description.
